(19) United States
(12) Patent Application Publication
(10) Pub. No.: US 2022/0411381 A1
(43) Pub. Date: Dec. 29, 2022

(54) PYRIMIDINEDIONE DERIVATIVES

(71) Applicant: Bayer Aktiengesellschaft, Leverkusen (DE)

(72) Inventors: Lea Aurelie BOUCHE, Basel (CH); Stefan KAULFUSS, Berlin (DE); Katja ZIMMERMANN, Düsseldorf (DE); Hartmut REHWINKEL, Berlin (DE); Roland NEUHAUS, Berlin (DE); Roman HILLIG, Berlin (DE); Duy NGUYEN, Berlin (DE); Judith GÜNTHER, Berlin (DE); Adelaide Clara FARIA ALVARES DE LEMOS, Berlin (DE)

(73) Assignee: Bayer Aktiengesellschaft, Leverkusen (DE)

(21) Appl. No.: 17/765,737

(22) PCT Filed: Sep. 25, 2020
(86) PCT No.: PCT/EP2020/076871

§ 371 Date: Mar. 31, 2022

(30) Foreign Application Priority Data
Oct. 1, 2019 (EP) 19200789.6

(51) Int. Cl. C07D 239/54; A61K 45/06; C07D 401/04; A61P 35/00

(52) U.S. Cl. CPC C07D 239/54; A61K 45/06; C07D 401/04; A61P 35/00

(57) ABSTRACT

The present invention covers pyrimidinedione derivatives of general formula (I): (I), in which R¹, R², R⁴, R⁵ and X are as defined herein, methods of preparing said compounds, intermediate compounds useful for preparing said compounds, pharmaceutical compositions and combinations comprising said compounds and the use of said compounds for manufacturing pharmaceutical compositions for the treatment or prophylaxis of diseases, in particular of cancer, as a sole agent or in combination with other active ingredients.

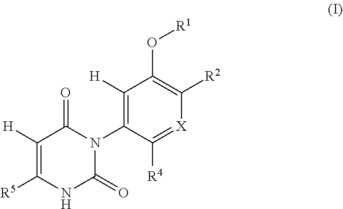

The present invention covers pyrimidinedione compounds of general formula (I) as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular of cancer, as a sole agent or in combination with other active ingredients.

### BACKGROUND

The branched chain amino acid transferases/transaminases (BOAT) are enzymes catalyzing the degradation of the branched chain amino acids (BCAA), valine, leucine and isoleucine to the respective branched chain α-keto acids (BCKA), alpha-ketoisovalerate, alpha-ketoisocaproate and alpha-keto-beta-methylvalerate. There are two isoenzymes known, BCAT1 (cytosolic form) and BCAT2 (mitochondrial form). While BCAT2 is considered to be ubiquitously expressed, the BCAT1 expression is only expressed in human adult brain, testis and uterus tissue and under pathological conditions, e.g. ischemia, nonalcoholic fatty liver disease and cancer.

In cancer BCAT1 is overexpressed in wild-type IDH gliomas and promotes cell proliferation, migration and tumor growth in xenografted mouse (Tönjes et al., Nat Med, 19(7), 901, 2013) and the BCAT1 expression increases the chemotherapy resistance in these tumors (Cho et al., Oncotarget, 25; 7(43), 69606, 2016) and is linked to clinical tumor aggressiveness (Panosyan et al., J Neurooncol, 128(1), 57, 2016). The BCAT1 activity is also required for the tumor growth of KRAS depending NSCLC tumors in animal studies (Mayers et al., Science, 353(6304), 1161, 2016). In anti-estrogen resistant end ERalpha-negative breast cancer the BCAT1 sustains the tumor growth (Thewes et al., Oncogene, 36(29), 4124, 2017). In Leukemia BCAT1 is also expressed and required in primary leukemic cells to transplant in mice and the BCAT1 action restricts the alpha-ketoglutarate levels in the tumors cells leading to DNA hypermethylation (Raffel et al., Nature, 551(7680), 384, 2017; Hattori et al., Nature, 545(7655), 500, 2017). Also in Prostate cancer BCAT1 is associated with chemoresistance (Zhu et al., Mol Carcinogen, 56(6), 1570, 2017). In Ovarian cancer the BCAT1 expression is associated with tumor progression and seems to control the global tumor cell metabolism (Wang et al., Oncotarget, 6(31), 31522, 2015). Furthermore the BCAT1 expression is an indicator of poor prognosis in patients with urothelial carcinomas of the upper urinary tract and urinary bladder (Chang et al., Histopthology, 68(4), 520, 2016) and also for patients with gastric cancer (Xu et al., Human Pathol, 75, 41, 2018). Additional, others could also show that the overexpression of BCAT1 induces cell proliferation, migration and invasion in nasopharyngeal carcinoma (Zhou et al., Mol Cancer, 12:53, 2013).

The present invention covers Pyrimidinedione compounds of general formula (I) which inhibit BOAT 1.

Compounds somewhat structurally related to those compounds of the present invention, such as 3-Phenyluracils, have been frequently disclosed as agrochemicals, such as defoliants, desiccants, plant growth regulators, insecticides, pesticides and, particularly, as herbicides, or as synthetic intermediates for the preparation of such agrochemicals, in numerous published patents and patent applications, namely US 2003/0187264, JP 2002/363170, JP 2002/383010, WO 2002/098227, WO 2002/098228, U.S. Pat. No. 6,333,296, WO 1998/041093, EP 1106607, US 2002/0013466, DE 19954312, WO 2001/034575, U.S. Pat. No. 6,121,201, WO 2000/002866, JP 2002/003480, WO 2011/137088, WO 2001/058883, WO 2001/039597, US 2004/0171488, EP 1101761, U.S. Pat. No. 6,339,155, WO 1998/027067, WO 1997/005116, WO 1997/001541, DE 19506202, WO 1996/016043, DE 4437197, WO 1996/007323, WO 1996/008151, WO 1995/032952, JP 05025144, JP 05025142, WO 1991/007393, WO 2000/078734, WO 1993/011669. WO 2001/034575 also specifically discloses three compounds which are not part of the present invention, as stated e.g. in claim 1, below, namely:
4-[2,6-Dioxo-4-(trifluoromethyl)-3,6-dihydropyrinnidin-1(2H)-yl]-5-fluoro-2-(2-fluorophenoxy)benzonitrile,
2-(2,4-Difluorophenoxy)-4-[2,6-dioxo-4-(trifluoromethyl)-3,6-dihydropyrimidin-1(2H)-yl]-5-fluorobenzonitrile, and
2-(2-Chlorophenoxy)-4-[2,6-dioxo-4-(trifluoromethyl)-3,6-dihydropyrimidin-1(2H)-yl]-5-fluorobenzonitrile.

However, the state of the art does not describe the pyrimidinedione compounds of general formula (I) of the present invention as described and defined herein, i.e. compounds having a pyrimidione core bearing combinations of e.g. R¹, R², X, R⁴ and R⁵ groups as disclosed herein. Particularly, the prior art does not disclose pyrimidinediones, 3-phenyluracils and related compounds as inhibitors of BCAT1, and also not in the context of the treatment and prophylaxis of cancer.

It has now been found, and this constitutes the basis of the present invention, that the compounds of the present invention have surprising and advantageous properties.

In particular, the compounds of the present invention have surprisingly been found to effectively inhibit BOAT 1 and may therefore be used for the treatment or prophylaxis of cancer, such as glioma, lung cancer, such as non-small-cell lung cancer (NSCLC), breast cancer, such as anti-estrogen resistant breast cancer and ERalpha-negative breast cancer, leukemia, prostate cancer, ovarian cancer, urothelial carcinoma, gastric cancer and nasopharyngeal carcinoma, for example, for the treatment and prophylaxis of cancer-induced cachexia. Particularly, the compounds of the present invention may be used for the treatment and prophylaxis of glioma and breast cancer; further, the compounds, as inhibitors of BOAT 1, may also be used for the treatment or prophylaxis for certain non-oncological diseases such as fibrosis.

### DESCRIPTION OF THE INVENTION

In accordance with a first aspect, the present invention covers compounds of general formula (I):

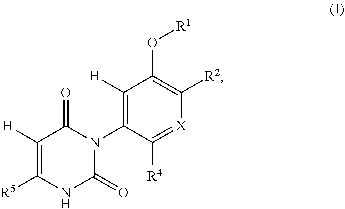

in which:

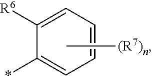

R¹ represents a group
   in which “*” represents the point of attachment to the rest of the molecule,
R² represents a chlorine atom or a cyano group,
X represents CR³ or N,
R³ represents a hydrogen atom,
R⁴ represents a hydrogen atom, a halogen atom or a group selected from hydroxy, C₁-C₃-alkyl, C₁-C₃-alkoxy and C₁-C₃-haloalkoxy,
or R³ and R⁴ together form a group —CH═CH—CH═CH—,
R⁵ represents a halogen atom or a group selected from C₁-C₃-haloalkyl, C₃-C₆-cycloalkyl, phenyl, —C(═O)NR⁸R⁹ and —S(═O)₂R¹⁰,
   said C₁-C₃-haloalkyl group being optionally substituted with one phenyl group, and said phenyl group being optionally substituted with one or two substituents selected independently from each other from a halogen atom and C₁-C₃-haloalkyl,
R⁶ represents a fluorine atom, a chlorine atom, or a methyl group,
R⁷ represents a halogen atom or a group selected from amino, cyano, C₁-C₃-alkyl or H₃C—C(═O)—,
R⁸ represents a hydrogen atom or a group selected from C₁-C₃-alkyl and C₁-C₃-haloalkyl,
R⁹ represents a hydrogen atom or a methyl group,
R¹⁰ represents a group selected from C₁-C₃-alkyl and phenyl, said phenyl group being optionally substituted with one or two substituents selected independently from each other from a halogen atom, C₁-C₃-alkyl, C₁-C₃-haloalkyl, C₁-C₃-alkoxy and cyano, and
n represents an integer 0, 1 or 2,

and stereoisomers, tautomers, N-oxides, hydrates, solvates, and salts thereof, and mixtures of same,

with the proviso that said compounds of general formula (I) are not:
4-[2,6-Dioxo-4-(trifluoromethyl)-3,6-dihydropyrimidin-1(2H)-yl]-5-fluoro-2-(2-fluorophenoxy)benzonitrile

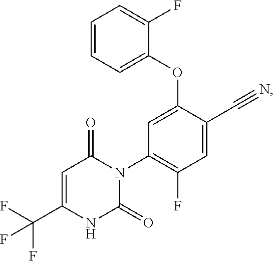 [SMILES: N#Cc1cc(F)c(-n2c(=O)cc(C(F)(F)F)[nH]c2=O)cc1Oc1ccccc1F]

2-(2,4-Difluorophenoxy)-4-[2,6-dioxo-4-(trifluoromethyl)-3,6-dihydropyrimidin-1(2H)-yl]-5-fluorobenzonitrile

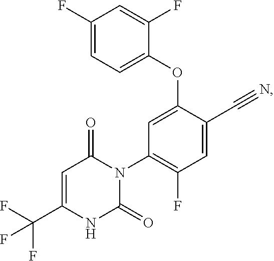 [SMILES: N#Cc1cc(F)c(-n2c(=O)cc(C(F)(F)F)[nH]c2=O)cc1Oc1ccc(F)cc1F]

and
2-(2-Chlorophenoxy)-4-[2,6-dioxo-4-(trifluoromethyl)-3,6-dihydropyrimidin-1(2H)-yl]-5-fluorobenzonitrile

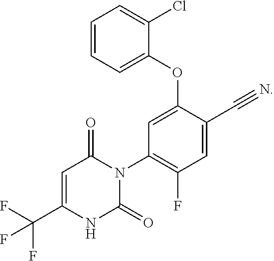 [SMILES: N#Cc1cc(F)c(-n2c(=O)cc(C(F)(F)F)[nH]c2=O)cc1Oc1ccccc1Cl]

### Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, or 3, in particular 1.

As used herein, an oxo substituent represents an oxygen atom, which is bound to a carbon atom or to a sulfur atom via a double bond.

The term “ring substituent” means a substituent attached to an aromatic or nonaromatic ring which replaces an available hydrogen atom on the ring.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom.

The term “C₁-C₄-alkyl” means a linear or branched, saturated, monovalent hydrocarbon group having 1, 2, 3 or 4 carbon atoms, e.g. a methyl, ethyl, propyl, isopropyl, butyl, sec-butyl, isobutyl, tert-butyl group, or an isomer thereof. Particularly, said group has 1, 2 or 3 carbon atoms (“C₁-C₃-alkyl”), e.g. a methyl, ethyl, n-propyl or isopropyl group.

The term “C₁-C₃-haloalkyl” means a linear or branched, saturated, monovalent hydrocarbon group in which the term “C₁-C₃-alkyl” is as defined supra, and in which one or more of the hydrogen atoms are replaced, identically or differently, with a halogen atom. Particularly, said halogen atom is a fluorine atom. Said C₁-C₃-haloalkyl group is, for example, fluoromethyl, difluoromethyl, chlorodifluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl.

The term “C₁-C₃-alkoxy” means a linear or branched, saturated, monovalent group of formula (C₁-C₃-alkyl)-O—, in which the term “C₁-C₃-alkyl” is as defined supra, e.g. a methoxy, ethoxy, n-propoxy, isopropoxy group, or an isomer thereof.

The term “C₁-C₃-haloalkoxy” means a linear or branched, saturated, monovalent C₁-C₃-alkoxy group, as defined supra, in which one or more of the hydrogen atoms is replaced, identically or differently, with a halogen atom. Particularly, said halogen atom is a fluorine atom. Said C₁-C₃-haloalkoxy group is, for example, fluoromethoxy, difluoromethoxy, trifluoromethoxy, 2,2,2-trifluoroethoxy or pentafluoroethoxy.

The term “C₃-C₆-cycloalkyl” means a saturated, monovalent, monocyclic hydrocarbon ring which contains 3, 4, 5 or 6 carbon atoms (“C₃-C₆-cycloalkyl”). Said C₃-C₆-cycloalkyl group is for example, a cyclopropyl, cyclobutyl, cyclopentyl or cyclohexyl group.

The term “C₁-C₃”, as used in the present text, e.g. in the context of the definition of “C₁-C₃-alkyl”, “C₁-C₃-haloalkyl”, “C₁-C₃-hydroxyalkyl”, “C₁-C₃-alkoxy” or “C₁-C₃-haloalkoxy” means an alkyl group having a finite number of carbon atoms of 1 to 3, i.e. 1, 2 or 3 carbon atoms.

The term “C₁-C₄”, as used in the present text, e.g. in the context of the definition of “C₁-C₄-alkyl, means an alkyl group having a finite number of carbon atoms of 1 to 4, i.e. 1, 2, 3 or 4 carbon atoms.

Further, as used herein, the term “C₃-C₆”, as used in the present text, e.g. in the context of the definition of “C₃-C₆-cycloalkyl”, means a cycloalkyl group having a finite number of carbon atoms of 3 to 6, i.e. 3, 4, 5 or 6 carbon atoms.

When a range of values is given, said range encompasses each value and sub-range within said range.

For example:

“C₁-C₄” encompasses C₁, C₂, C₃, C₄, C₁-C₄, C₁-C₃, C₁-C₂, C₂-C₄, C₂-C₃ and C₃-C₄;

“C₁-C₃” encompasses C₁, C₂, C₃, C₁-C₃, C₁-C₂ and C₂-C₃;

“C₃-C₆” encompasses C₃, C₄, C₅, C₆, C₃-C₄, C₃-C₅, C₃-C₄, C₄-C₆, C₄-C₅, and C₅-C₆; As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)-sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butyl-phenyl)sulfonyl]oxy and [(4-methoxyphenyl)sulfonyl]oxy.

As used herein, the term “dipolar aprotic solvent” means a solvent selected from dimethylsulfoxide, diethylsulfoxide, N,N-dimethylformamide, N,N-dimethylacetamide, N,N-diethylformamide, N,N-diethylacetamide, 1-methyl-2-pyrrolidinone, 1-ethyl-2-pyrrolidinone, 1-methyl-2-piperidinone and 1-ethyl-2-piperidinone, or mixtures thereof. Particularly, said dipolar aprotic solvent dimethylsulfoxide, N,N-dimethylformamide, N,N-dimethylacetamide or 1-methyl-2-pyrrolidinone.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacal., 2000, 169, 102). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P450.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

In certain compounds of the present invention, optionally asymmetry may be present due to restricted rotation about a given bond, for example, the central bond adjoining two substituted aromatic rings of the specified compounds. Particularly, dependent on the sterical demands of the substituent in position R⁴, e.g. if R⁴ is different from hydrogen, compounds of the present invention can exist as atropisomers, as shown in Scheme 1. Atropisomers represent a subclass of conformers which arise from restricted rotation around a single bond. The conformers (called atropisomers) can be isolated as separated species (IUPAC Gold book, http://goldbook.iupac.orq/A00511.html; Pure and Appl. Chem., 2009, 68, 2193-2222). This induced chirality belongs to the axial type of chirality. Hence, compounds featuring said atropisomerism and an additional asymmetric centre can exist as diasteromeric mixtures as described supra.

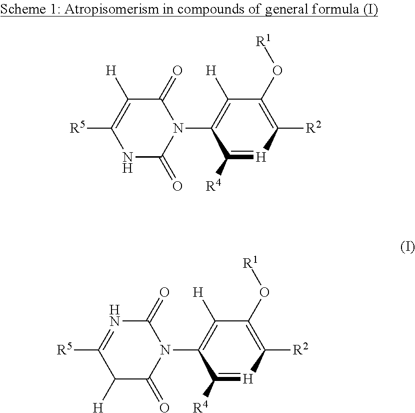

Several related compounds with the related type of atropisomerism have been already described as observed in the literature (G. Bringmann et al., Chem. Rev., 2011, 111, 563-639). For examples of atropisomers in drug discovery, see the mini-review from J. Clayden, S. R. Laplante et al.: Angew. Chem. Int. Ed. 2009, 48, 6398-6401 and also: S. R. LaPlante, P. J. Edwards et al., J. Med. Chem. 2011, 54, 7005-7022; and a recent article by T. Nguyen in Chemistry & Engineering News 2018, vol. 96, issue 33.

Further, the compounds of the present invention may optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple elements of asymmetry, such as axial chirality and asymmetric centres.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art, examples of which can be found in the experimental section. If the atropisomers were separated, said atropisomers are being referred to as “atrop 1” (for atropisomer 1) and “atrop 2” (for atropisomer 2), subsequent to the respective compound name. Names without any such indication but still naming a compound showing atropisomerism is to be understood to include both atropisomers which were not separated.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it is possible for the compounds of the present invention to exist as tautomers. For example, the pyrimidinedione present in the compounds of the present invention can also exist as two hydroxypyrimidinone tautomers, or as a mixture in any amount of said tautomers, namely:

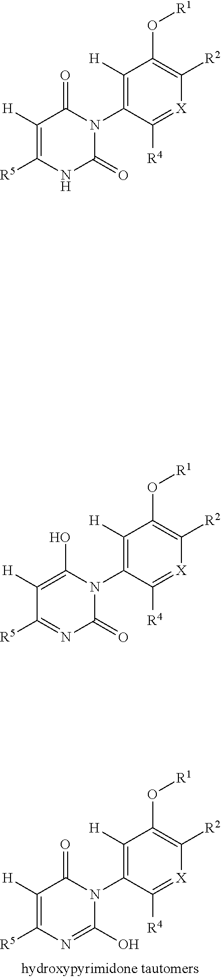

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Nat”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

In accordance with a second embodiment of the first aspect, the present invention covers compounds of general formula (I), supra, in which:
R¹ represents a group

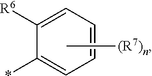

in which “*” represents the point of attachment to the rest of the molecule,
R² represents a chlorine atom or a cyano group,
X represents CR³,
R³ represents a hydrogen atom,
R⁴ represents a hydrogen atom, a halogen atom or a group selected from hydroxy, C₁-C₂-alkyl, C₁-C₃-alkoxy and C₁-C₂-fluoroalkoxy,
or R³ and R⁴ together form a group —CH═CH—CH═CH—
R⁵ represents a halogen atom or a group selected from C₁-C₂-haloalkyl, C₃-C₄-cycloalkyl, phenyl, —C(═O)NR⁸R⁹ and —S(═O)₂R¹⁰,
   said C₁-C₂-haloalkyl group being optionally substituted with one phenyl group,
   and said phenyl group being optionally substituted with one or two substituents selected independently from each other from a fluorine atom, a chlorine atom, a bromine atom and C₁-C₂-fluoroalkyl,
R⁶ represents a fluorine atom, a chlorine atom, or a methyl group,
R⁷ represents a halogen atom or a group selected from amino, cyano, C₁-C₃-alkyl or H₃C—C(═O)—,
R⁸ represents a hydrogen atom or a group selected from C₁-C₂-alkyl and C₁-C₂-fluoroalkyl,
R⁹ represents a hydrogen atom,
R¹⁰ represents a group selected from C₁-C₂-alkyl and phenyl, said phenyl group being optionally substituted with one or two substituents selected independently from each other from a fluorine atom, a chlorine atom, a bromine atom, C₁-C₂-alkyl, C₁-C₂-fluoroalkyl and C₁-C₂-alkoxy, and
n represents an integer 0, 1 or 2,

and stereoisomers, tautomers, N-oxides, hydrates, solvates, and salts thereof, and mixtures of same,

with the proviso that said compounds of general formula (I) are not:
4-[2,6-Dioxo-4-(trifluoromethyl)-3,6-dihydropyrimidin-1(2H)-yl]-5-fluoro-2-(2-fluorophenoxy)benzonitrile,
2-(2,4-Difluorophenoxy)-4-[2,6-dioxo-4-(trifluoromethyl)-3,6-dihydropyrimidin-1(2H)-yl]-5-fluorobenzonitrile, and
2-(2-Chlorophenoxy)-4-[2,6-dioxo-4-(trifluoromethyl)-3,6-dihydropyrimidin-1(2H)-yl]-5-fluorobenzonitrile.

In accordance with a third embodiment of the first aspect, the present invention covers compounds of general formula (I), supra, in which:
R¹ represents a group

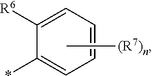

in which “*” represents the point of attachment to the rest of the molecule,
R² represents a chlorine atom or a cyano group,
X represents CR³,
R³ and R⁴ together form a group —CH═CH—CH═CH—
R⁵ represents a halogen atom or a group selected from C₁-C₂-haloalkyl, C₃-C₄-cycloalkyl, phenyl, —C(═O)NR⁸R⁹ and —S(═O)₂R¹⁰,
   said C₁-C₂-haloalkyl group being optionally substituted with one phenyl group,
   and said phenyl group being optionally substituted with one or two substituents selected independently from each other from a fluorine atom, a chlorine atom, a bromine atom and C₁-C₂-fluoroalkyl,
R⁶ represents a fluorine atom, a chlorine atom, or a methyl group,
R⁷ represents a halogen atom or a group selected from amino, cyano, C₁-C₃-alkyl or H₃C—C(═O)—,
R⁸ represents a hydrogen atom or a group selected from C₁-C₂-alkyl and C₁-C₂-fluoroalkyl,
R⁹ represents a hydrogen atom,
R¹⁰ represents a group selected from C₁-C₂-alkyl and phenyl, said phenyl group being optionally substituted with one or two substituents selected independently from each other from a fluorine atom, a chlorine atom, a bromine atom, C₁-C₂-alkyl, C₁-C₂-fluoroalkyl and C₁-C₂-alkoxy, and
n represents an integer 0, 1 or 2,

and stereoisomers, tautomers, N-oxides, hydrates, solvates, and salts thereof, and mixtures of same.

In accordance with a fourth embodiment of the first aspect, the present invention covers compounds of general formula (I), supra, in which:
R¹ represents a group

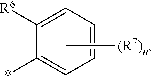

in which “*” represents the point of attachment to the rest of the molecule,
R² represents a chlorine atom or a cyano group,
X represents CR³,
R³ represents a hydrogen atom,
R⁴ represents a halogen atom or a group selected from methyl, methoxy, trifluoromethoxy and n-propoxy,
or R³ and R⁴ together form a group —CH═CH—CH═CH—,
R⁵ represents a bromine atom or a group selected from difluoromethyl, (phenyl)-difluoromethyl, chlorodifluoromethyl, trifluoromethyl, cyclopropyl, methanesulfonyl and benzenesulfonyl,
R⁶ represents a fluorine atom, a chlorine atom, or a methyl group,
R⁷ represents a halogen atom or a group selected from amino, cyano, C₁-C₃-alkyl or H₃C—C(═O)—, and
n represents an integer 0, 1 or 2, and stereoisomers, tautomers, N-oxides, hydrates, solvates, and salts thereof, and mixtures of same,
with the proviso that said compounds of general formula (I) are not:
4-[2,6-Dioxo-4-(trifluoromethyl)-3,6-dihydropyrimidin-1(2H)-yl]-5-fluoro-2-(2-fluorophenoxy)benzonitrile,
2-(2,4-Difluorophenoxy)-4-[2,6-dioxo-4-(trifluoromethyl)-3,6-dihydropyrimidin-1(2H)-yl]-5-fluorobenzonitrile, and
2-(2-Chlorophenoxy)-4-[2,6-dioxo-4-(trifluoromethyl)-3,6-dihydropyrimidin-1(2H)-yl]-5-fluorobenzonitrile.

In accordance with a fifth embodiment of the first aspect, the present invention covers compounds of general formula (I), supra, in which:
R¹ represents a group

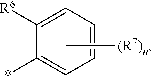

in which “*” represents the point of attachment to the rest of the molecule,
R² represents a chlorine atom or a cyano group,
X represents CR³,
R³ and R⁴ together form a group —CH═CH—CH═CH—,
R⁵ represents a bromine atom or a group selected from difluoromethyl, (phenyl)-difluoromethyl, chlorodifluoromethyl, trifluoromethyl, cyclopropyl, methanesulfonyl and benzenesulfonyl,
R⁶ represents a fluorine atom, a chlorine atom, or a methyl group,
R⁷ represents a halogen atom or a group selected from amino, cyano, C₁-C₃-alkyl or H₃C—C(═O)—, and
n represents an integer 0, 1 or 2,

and stereoisomers, tautomers, N-oxides, hydrates, solvates, and salts thereof, and mixtures of same.

In accordance with a sixth embodiment of the first aspect, the present invention covers compounds:
2-(2,6-Dimethylphenoxy)-4-[2,6-dioxo-4-(trifluoromethyl)-3,6-dihydropyrimidin-1(2H)-yl]-5-fluorobenzonitrile,
2-(2-Chloro-6-methylphenoxy)-4-[2,6-dioxo-4-(trifluoromethyl)-3,6-dihydropyrimidin-1(2H)-yl]-5-fluorobenzonitrile,
4-(4-Bromo-2,6-dioxo-3,6-dihydropyrimidin-1(2H)-yl)-5-fluoro-2-(2-methylphenoxy)benzonitrile,
4-[2,6-Dioxo-4-(trifluoromethyl)-3,6-dihydropyrimidin-1(2H)-yl]-5-methoxy-2-(2-methylphenoxy)benzonitrile (rac),
4-[2,6-Dioxo-4-(trifluoromethyl)-3,6-dihydropyrinnidin-1(2H)-yl]-5-methoxy-2-(2-methylphenoxy)benzonitrile (atrop 1),
4-[2,6-Dioxo-4-(trifluoromethyl)-3,6-dihydropyrimidin-1(2H)-yl]-5-methoxy-2-(2-methylphenoxy)benzonitrile (atrop 2),
4-[2,6-Dioxo-4-(trifluoromethyl)-3,6-dihydropyrimidin-1(2H)-yl]-5-methyl-2-(2-methylphenoxy)benzonitrile (rac),
4-[2,6-Dioxo-4-(trifluoromethyl)-3,6-dihydropyrimidin-1(2H)-yl]-5-methyl-2-(2-methylphenoxy)benzonitrile (atrop 1),
4-[2,6-Dioxo-4-(trifluoromethyl)-3,6-dihydropyrimidin-1(2H)-yl]-5-methyl-2-(2-methylphenoxy)benzonitrile (atrop 2),
2-(2-Chlorophenoxy)-4-[2,6-dioxo-4-(trifluoromethyl)-3,6-dihydropyrimidin-1(2H)-yl]-5-methylbenzonitrile (rac),
5-Chloro-4-[2,6-dioxo-4-(trifluoromethyl)-3,6-dihydropyrinnidin-1(2H)-yl]-2-(2-methylphenoxy)benzonitrile (rac),
5-Chloro-4-[2,6-dioxo-4-(trifluoromethyl)-3,6-dihydropyrimidin-1(2H)-yl]-2-(2-methylphenoxy)benzonitrile (atrop 1),
5-Chloro-4-[2,6-dioxo-4-(trifluoromethyl)-3,6-dihydropyrimidin-1(2H)-yl]-2-(2-methylphenoxy)benzonitrile (atrop 2),
4-[2,6-Dioxo-4-(trifluoromethyl)-3,6-dihydropyrinnidin-1(2H)-yl]-5-iodo-2-(2-methylphenoxy)benzonitrile (rac),
2-(2-Chlorophenoxy)-4-[2,6-dioxo-4-(trifluoromethyl)-3,6-dihydropyrimidin-1(2H)-yl]-5-methoxybenzonitrile (rac),
2-(2-Chlorophenoxy)-4-[2,6-dioxo-4-(trifluoromethyl)-3,6-dihydropyrimidin-1(2H)-yl]-5-methoxybenzonitrile (atrop 1),
2-(2-Chlorophenoxy)-4-[2,6-dioxo-4-(trifluoromethyl)-3,6-dihydropyrimidin-1(2H)-yl]-5-methoxybenzonitrile (atrop 2),
5-Bromo-4-[2,6-dioxo-4-(trifluoromethyl)-3,6-dihydropyrimidin-1(2H)-yl]-2-(2-methylphenoxy)benzonitrile (rac),
4-[4-Bromo-2,6-dioxo-3,6-dihydropyrinnidin-1(2H)-yl]-5-methoxy-2-(2-methylphenoxy)benzonitrile (rac),
4-[4-Bromo-2,6-dioxo-3,6-dihydropyrinnidin-1(2H)-yl]-5-methoxy-2-(2-methylphenoxy)benzonitrile (atrop 1),
4-[4-Bromo-2,6-dioxo-3,6-dihydropyrimidin-1(2H)-yl]-5-methoxy-2-(2-methylphenoxy)benzonitrile (atrop 2),
4-[4-(Difluoromethyl)-2,6-dioxo-3,6-dihydropyrinnidin-1(2H)-yl]-5-methoxy-2-(2-methylphenoxy)benzonitrile (rac),
4-(4-Cyclopropyl-2,6-dioxo-3,6-dihydropyrimidin-1(2H)-yl)-5-methoxy-2-(2-methylphenoxy)benzonitrile (rac),
4-{4-[Difluoro(phenyl)methyl]-2,6-dioxo-3,6-dihydropyrimidin-1(2H)-yl}-5-methoxy-2-(2-methylphenoxy)benzonitrile (rac),
4-{4-[Chloro(difluoro)methyl]-2,6-dioxo-3,6-dihydropyrimidin-1(2H)-yl}-5-methoxy-2-(2-methylphenoxy)benzonitrile (rac),
5-Chloro-4-{4-[chloro(difluoro)methyl]-2,6-dioxo-3,6-dihydropyrimidin-1(21-)-yl}-2-(2-methylphenoxy)benzonitrile (rac),
3-[4-Chloro-2-methoxy-5-(2-methylphenoxy)phenyl]-6-(trifluoromethyl)pyrimidine-2,4(1H,3H)-dione (rac),
3-[4-Chloro-2-methoxy-5-(2-methylphenoxy)phenyl]-6-(difluoromethyl)pyrimidine-2,4(1H,3H)-dione (rac),
3-[4-Chloro-3-(2-methylphenoxy)naphthalen-1-yl]-6-[difluoro(phenyl)methyl]pyrimidine-2,4(1H,3H)-dione (rac),
3-[4-chloro-3-(2-methylphenoxy)naphthalen-1-yl]-6-[difluoro(phenyl)methyl]pyrimidine-2,4(1H,3H)-dione (atrop 1),
3-[4-chloro-3-(2-methylphenoxy)naphthalen-1-yl]-6-[difluoro(phenyl)methyl]pyrimidine-2,4(1H,3H)-dione (atrop 2),
3-[4-Chloro-3-(2-methylphenoxy)naphthalen-1-yl]-6-(trifluoromethyl)pyrimidine-2,4(1H,3H)-dione (rac),
3-[4-Chloro-3-(2-methylphenoxy)naphthalen-1-yl]-6-(trifluoromethyl)pyrimidine-2,4(1H,3H)-dione (atrop 1),
3-[4-Chloro-3-(2-methylphenoxy)naphthalen-1-yl]-6-(trifluoromethyl)pyrimidine-2,4(1H,3H)-dione (atrop 2),
4-[2,6-Dioxo-4-(trifluoromethyl)-3,6-dihydropyrimidin-1(2H)-yl]-2-(2-methylphenoxy)naphthalene-1-carbonitrile (rac),
4-[2,6-Dioxo-4-(trifluoromethyl)-3,6-dihydropyrimidin-1(2H)-yl]-2-(2-methylphenoxy)naphthalene-1-carbonitrile (atrop 1),
4-[2,6-Dioxo-4-(trifluoromethyl)-3,6-dihydropyrimidin-1(2H)-yl]-2-(2-methylphenoxy)naphthalene-1-carbonitrile (atrop 2),
5-Chloro-2-(2,6-dimethylphenoxy)-4-(2,6-dioxo-4-(trifluoromethyl)-2,3-dihydropyrimidin-1(6H)-yl)benzonitrile (rac),
4-(2,6-Dioxo-4-(trifluoromethyl)-2,3-dihydropyrimidin-1(6H)-yl)-5-propoxy-2-(o-tolyloxy)benzonitrile (rac),
4-{4-[Chloro(difluoro)methyl]-2,6-dioxo-3,6-dihydropyrimidin-1(2H)-yl}-5-fluoro-2-(2-methylphenoxy)benzonitrile,
4-[2,6-Dioxo-4-(trifluoromethyl)-3,6-dihydropyrimidin-1(2H)-yl]-2-(2-methylphenoxy)benzonitrile,
4-(2,6-Dioxo-4-phenyl-3,6-dihydropyrimidin-1(2H)-yl)-5-methoxy-2-(2-methylphenoxy)benzonitrile (rac),
4-(2,6-Dioxo-4-phenyl-3,6-dihydropyrimidin-1(2H)-yl)-5-methoxy-2-(2-methylphenoxy)benzonitrile (atrop 1),
4-(2,6-Dioxo-4-phenyl-3,6-dihydropyrimidin-1(2H)-yl)-5-methoxy-2-(2-methylphenoxy)benzonitrile (atrop 2),
4-[2,6-Dioxo-4-(trifluoromethyl)-3,6-dihydropyrimidin-1(2H)-yl]-5-fluoro-2-(2-methylphenoxy)benzonitrile,
2-(2-Chlorophenoxy)-4-[2,6-dioxo-4-(trifluoromethyl)-3,6-dihydropyrimidin-1(2H)-yl]benzonitrile,
5-[2,6-Dioxo-4-(trifluoromethyl)-3,6-dihydropyrimidin-1(2H)-yl]-3-(2-methylphenoxy)pyridine-2-carbonitrile,
2-(2,6-Dimethylphenoxy)-4-[2,6-dioxo-4-(trifluoromethyl)-3,6-dihydropyrimidin-1(2H)-yl]benzonitrile,
4-(2,6-Dioxo-4-phenyl-3,6-dihydropyrimidin-1(2H)-yl)-5-fluoro-2-(2-methylphenoxy)benzonitrile,
3-[4-Chloro-5-(2-chlorophenoxy)-2-methoxyphenyl]-6-(trifluoromethyl)pyrimidine-2,4(1H,3H)-dione (rac),
4-[2,6-Dioxo-4-(pentafluoroethyl)-3,6-dihydropyrimidin-1(2H)-yl]-5-methoxy-2-(2-methylphenoxy)benzonitrile (rac),
4-[2,6-dioxo-4-(pentafluoroethyl)-3,6-dihydropyrimidin-1(2H)-yl]-5-fluoro-2-(2-methylphenoxy)benzonitrile,
5-Chloro-4-{4-[difluoro(phenyl)methyl]-2,6-dioxo-3,6-dihydropyrimidin-1(2H)-yl}-2-(2-methylphenoxy)benzonitrile (rac),
4-[2,6-Dioxo-4-(trifluoromethyl)-3,6-dihydropyrimidin-1(2H)-yl]-5-hydroxy-2-(2-methylphenoxy)benzonitrile (rac),
4-[2,6-Dioxo-4-(trifluoromethyl)-3,6-dihydropyrimidin-1(2H)-yl]-2-(2-methylphenoxy)-5-(trifluoromethoxy)benzonitrile (rac),
4-{4-[Difluoro(phenyl)methyl]-2,6-dioxo-3,6-dihydropyrimidin-1(2H)-yl}-5-fluoro-2-(2-methylphenoxy)benzonitrile,
4-{4-[Difluoro(phenyl)methyl]-2,6-dioxo-3,6-dihydropyrinnidin-1(2H)-yl}-2-(2,6-dimethylphenoxy)-5-fluorobenzonitrile,
2-(2-Acetyl-4,6-dimethylphenoxy)-4-[2,6-dioxo-4-(trifluoromethyl)-3,6-dihydropyrimidin-1(2H)-yl]-5-fluorobenzonitrile,
4-[2,6-Dioxo-4-(trifluoromethyl)-3,6-dihydropyrinnidin-1(2H)-yl]-2-(2-ethyl-6-methylphenoxy)-5-fluorobenzonitrile,
4-[2,6-Dioxo-4-(trifluoromethyl)-3,6-dihydropyrimidin-1(2H)-yl]-5-fluoro-2-(2,3,6-trimethylphenoxy)benzonitrile,
2-(4-Chloro-2,6-dimethylphenoxy)-4-[2,6-dioxo-4-(trifluoromethyl)-3,6-dihydropyrimidin-1(2H)-yl]-5-fluorobenzonitrile,
4-[2,6-Dioxo-4-(trifluoromethyl)-3,6-dihydropyrimidin-1(2H)-yl]-5-fluoro-2-(2,4,6-trimethylphenoxy)benzonitrile,
2-(2,6-Difluorophenoxy)-4-[2,6-dioxo-4-(trifluoromethyl)-3,6-dihydropyrimidin-1(2H)-yl]-5-fluorobenzonitrile,
2-(2-Amino-6-fluorophenoxy)-4-[2,6-dioxo-4-(trifluoromethyl)-3,6-dihydropyrimidin-1(2H)-yl]-5-fluorobenzonitrile,
2-(2-Amino-6-methylphenoxy)-4-[2,6-dioxo-4-(trifluoromethyl)-3,6-dihydropyrimidin-1(2H)-yl]-5-fluorobenzonitrile,
2-(2,4-Dimethylphenoxy)-4-[2,6-dioxo-4-(trifluoromethyl)-3,6-dihydropyrimidin-1(2H)-yl]-5-fluorobenzonitrile,
2-(2,3-Dimethylphenoxy)-4-[2,6-dioxo-4-(trifluoromethyl)-3,6-dihydropyrimidin-1(2H)-yl]-5-fluorobenzonitrile,
4-{2,6-Dioxo-4-[4-(trifluoromethyl)phenyl]-3,6-dihydropyrimidin-1(2H)-yl}-5-fluoro-2-(2-methylphenoxy)benzonitrile,
4-{2,6-Dioxo-4-[4-(trifluoromethyl)phenyl]-3,6-dihydropyrimidin-1(2H)-yl}-5-methoxy-2-(2-methylphenoxy)benzonitrile (rac),
4-[4-(3-Chlorophenyl)-2,6-dioxo-3,6-dihydropyrimidin-1(2H)-yl]-5-fluoro-2-(2-methylphenoxy)benzonitrile,
1-[4-Cyano-2-fluoro-5-(2-methylphenoxy)phenyl]-2,6-dioxo-N-(2,2,2-trifluoroethyl)-1,2,3,6-tetrahydropyrimidine-4-carboxamide,
4-(4-Cyclopropyl-2,6-dioxo-3,6-dihydropyrimidin-1(2H)-yl)-5-fluoro-2-(2-methylphenoxy)benzonitrile,
1-[4-Cyano-2-methoxy-5-(2-methylphenoxy)phenyl]-2,6-dioxo-1,2,3,6-tetrahydropyrimidine-4-carboxamide (rac),
4-[4-(Benzenesulfonyl)-2,6-dioxo-3,6-dihydropyrimidin-1(2H)-yl]-5-fluoro-2-(2-methylphenoxy)benzonitrile,
5-Fluoro-4-[4-(methanesulfonyl)-2,6-dioxo-3,6-dihydropyrimidin-1 (2H)-yl]-2-(2-methylphenoxy)benzonitrile,
4-[4-(Benzenesulfonyl)-2,6-dioxo-3,6-dihydropyrimidin-1(2H)-yl]-5-methoxy-2-(2-methylphenoxy)benzonitrile (rac),
4-[4-(Benzenesulfonyl)-2,6-dioxo-3,6-dihydropyrimidin-1(2H)-yl]-5-methoxy-2-(2-methylphenoxy)benzonitrile (atrop 1),
4-[4-(Benzenesulfonyl)-2,6-dioxo-3,6-dihydropyrimidin-1(2H)-yl]-5-methoxy-2-(2-methylphenoxy)benzonitrile (atrop 2),
4-[4-(Methanesulfonyl)-2,6-dioxo-3,6-dihydropyrimidin-1 (2H)-yl]-5-methoxy-2-(2-methylphenoxy)benzonitrile (rac),
4-[4-(Methanesulfonyl)-2,6-dioxo-3,6-dihydropyrimidin-1 (2H)-yl]-5-methoxy-2-(2-methylphenoxy)benzonitrile (atrop 1), and
4-[4-(Methanesulfonyl)-2,6-dioxo-3,6-dihydropyrimidin-1 (2H)-yl]-5-methoxy-2-(2-methylphenoxy)benzonitrile (atrop 2),
and stereoisomers, tautomers, N-oxides, hydrates, solvates, and salts thereof, and mixtures of same.

In accordance with a seventh embodiment of the first aspect, the present invention covers compounds of general formula (I), supra, in which:
R¹ represents a group

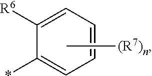

in which “*” represents the point of attachment to the rest of the molecule,
R² represents a chlorine atom or a cyano group,
X represents CR³ or N,
R³ represents a hydrogen atom,
R⁴ represents a hydrogen atom, a chlorine atom, a bromine atom, an iodine atom, or a group selected from hydroxy, C₁-C₃-alkyl, C₁-C₃-alkoxy and C₁-C₃-haloalkoxy,
or R³ and R⁴ together form a group —CH═CH—CH═CH—,
R⁵ represents a halogen atom or a group selected from C₁-C₃-haloalkyl, C₃-C₆-cycloalkyl, phenyl, —C(═O)NR⁸R⁹ and —S(═O)₂R¹⁰,
   said C₁-C₃-haloalkyl group being optionally substituted with one phenyl group,
   and said phenyl group being optionally substituted with one or two substituents selected independently from each other from a halogen atom and C₁-C₃-haloalkyl,
R⁶ represents a fluorine atom, a chlorine atom, or a methyl group,
R⁷ represents a halogen atom or a group selected from amino, cyano, C₁-C₃-alkyl or H₃C—C(═O)—,
R⁸ represents a hydrogen atom or a group selected from C₁-C₃-alkyl and C₁-C₃-haloalkyl,
R⁹ represents a hydrogen atom or a methyl group,
R¹⁰ represents a group selected from C₁-C₃-alkyl and phenyl, said phenyl group being optionally substituted with one or two substituents selected independently from each other from a halogen atom, C₁-C₃-alkyl, C₁-C₃-haloalkyl, C₁-C₃-alkoxy and cyano, and
n represents an integer 0, 1 or 2,

and stereoisomers, tautomers, N-oxides, hydrates, solvates, and salts thereof, and mixtures of same.

In accordance with a eighth embodiment of the first aspect, the present invention covers compounds of general formula (I), supra, in which:
R¹ represents a group

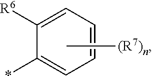

in which “*” represents the point of attachment to the rest of the molecule,
R² represents a chlorine atom,
X represents CR³ or N,
R³ represents a hydrogen atom,
R⁴ represents a hydrogen atom, a halogen atom or a group selected from hydroxy, C₁-C₃-alkyl, C₁-C₃-alkoxy and C₁-C₃-haloalkoxy,
or R³ and R⁴ together form a group —CH═CH—CH═CH—,
R⁵ represents a halogen atom or a group selected from C₁-C₃-haloalkyl, C₃-C₆-cycloalkyl, phenyl, —C(═O)NR⁸R⁹ and —S(═O)₂R¹⁰,
   said C₁-C₃-haloalkyl group being optionally substituted with one phenyl group,
   and said phenyl group being optionally substituted with one or two substituents selected independently from each other from a halogen atom and C₁-C₃-haloalkyl,
R⁶ represents a fluorine atom, a chlorine atom, or a methyl group,
R⁷ represents a halogen atom or a group selected from amino, cyano, C₁-C₃-alkyl or H₃C—C(═O)—,
R⁸ represents a hydrogen atom or a group selected from C₁-C₃-alkyl and C₁-C₃-haloalkyl,
R⁹ represents a hydrogen atom or a methyl group,
R¹⁰ represents a group selected from C₁-C₃-alkyl and phenyl, said phenyl group being optionally substituted with one or two substituents selected independently from each other from a halogen atom, C₁-C₃-alkyl, C₁-C₃-haloalkyl, C₁-C₃-alkoxy and cyano, and
n represents an integer 0, 1 or 2,

and stereoisomers, tautomers, N-oxides, hydrates, solvates, and salts thereof, and mixtures of same.

In accordance with an ninth embodiment of the first aspect, the present invention covers compounds of general formula (I), supra, in which:
R¹ represents a group

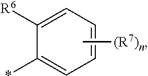

in which “*” represents the point of attachment to the rest of the molecule,
R² represents a chlorine atom or a cyano group,
X represents CR³,
R³ represents a hydrogen atom,
R⁴ represents a hydrogen atom, a halogen atom or a group selected from hydroxy, C₁-C₂-alkyl, C₁-C₃-alkoxy and C₁-C₂-fluoroalkoxy,
R⁵ represents a halogen atom or a group selected from C₁-C₂-haloalkyl, C₃-C₄-cycloalkyl, phenyl, —C(═O)NR⁸R⁹ and —S(═O)₂R¹⁰,
   said C₁—O₂-haloalkyl group being optionally substituted with one phenyl group,
   and said phenyl group being optionally substituted with one or two substituents selected independently from each other from a fluorine atom, a chlorine atom, a bromine atom and C₁-C₂-fluoroalkyl,
R⁶ represents a fluorine atom, a chlorine atom, or a methyl group,
R⁷ represents a halogen atom or a group selected from amino, cyano, C₁-C₃-alkyl or H₃C—C(═O)—,
R⁸ represents a hydrogen atom or a group selected from C₁-C₂-alkyl and C₁-C₂-fluoroalkyl,
R⁹ represents a hydrogen atom,
R¹⁰ represents a group selected from C₁-C₂-alkyl and phenyl, said phenyl group being optionally substituted with one or two substituents selected independently from each other from a fluorine atom, a chlorine atom, a bromine atom, C₁-C₂-alkyl, C₁-C₂-fluoroalkyl and C₁-C₂-alkoxy, and
n represents an integer 0, 1 or 2,

and stereoisomers, tautomers, N-oxides, hydrates, solvates, and salts thereof, and mixtures of same,

with the proviso that said compounds of general formula (I) are not:
4-[2,6-Dioxo-4-(trifluoromethyl)-3,6-dihydropyrimidin-1(2H)-yl]-5-fluoro-2-(2-fluorophenoxy)benzonitrile,
2-(2,4-Difluorophenoxy)-4-[2,6-dioxo-4-(trifluoromethyl)-3,6-dihydropyrimidin-1(2H)-yl]-5-fluorobenzonitrile, and
2-(2-Chlorophenoxy)-4-[2,6-dioxo-4-(trifluoromethyl)-3,6-dihydropyrimidin-1(2H)-yl]-5-fluorobenzonitrile.

In accordance with a tenth embodiment of the first aspect, the present invention covers compounds of general formula (I), supra, in which:
R¹ represents a group

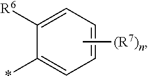

in which “*” represents the point of attachment to the rest of the molecule,
R² represents a chlorine atom or a cyano group,
X represents CR³,
R³ represents a hydrogen atom,
R⁴ represents a halogen atom or a group selected from methyl, methoxy, trifluoromethoxy and n-propoxy,
R⁵ represents a bromine atom or a group selected from difluoromethyl, (phenyl)-difluoromethyl, chlorodifluoromethyl, trifluoromethyl, cyclopropyl, methanesulfonyl and benzenesulfonyl,
R⁶ represents a fluorine atom, a chlorine atom, or a methyl group,
R⁷ represents a halogen atom or a group selected from amino, cyano, C₁-C₃-alkyl or H₃C—C(═O)—, and
n represents an integer 0, 1 or 2,

and stereoisomers, tautomers, N-oxides, hydrates, solvates, and salts thereof, and mixtures of same,

with the proviso that said compounds of general formula (I) are not:
4-[2,6-Dioxo-4-(trifluoromethyl)-3,6-dihydropyrimidin-1(2H)-yl]-5-fluoro-2-(2-fluorophenoxy)benzonitrile,
2-(2,4-Difluorophenoxy)-4-[2,6-dioxo-4-(trifluoromethyl)-3,6-dihydropyrimidin-1(2H)-yl]-5-fluorobenzonitrile, and
2-(2-Chlorophenoxy)-4-[2,6-dioxo-4-(trifluoromethyl)-3,6-dihydropyrimidin-1(2H)-yl]-5-fluorobenzonitrile.

Further Embodiments of the First Aspect of the Present Invention:

In a further embodiment of the first aspect, the present invention covers compounds of formula (I), supra, in which:

R¹ represents a group selected from:

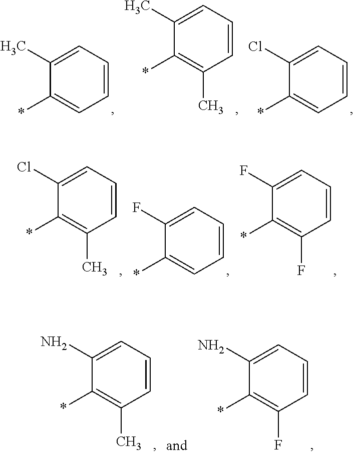

in which “*” represents the point of attachment to the rest of the molecule, and stereoisomers, tautomers, N-oxides, hydrates, solvates, and salts thereof, and mixtures of same,

with the proviso that said compounds of general formula (I) are not:
4-[2,6-Dioxo-4-(trifluoromethyl)-3,6-dihydropyrimidin-1(2H)-yl]-5-fluoro-2-(2-fluorophenoxy)benzonitrile, and
2-(2-Chlorophenoxy)-4-[2,6-dioxo-4-(trifluoromethyl)-3,6-dihydropyrimidin-1(2H)-yl]-5-fluorobenzonitrile.

In a further embodiment of the first aspect, the present invention covers compounds of formula (I), supra, in which:

R¹ represents a group selected from:

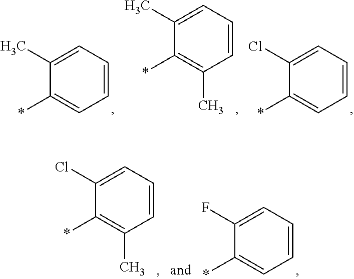

in which “*” represents the point of attachment to the rest of the molecule, and stereoisomers, tautomers, N-oxides, hydrates, solvates, and salts thereof, and mixtures of same, with the proviso that said compounds of general formula (I) are not:
4-[2,6-Dioxo-4-(trifluoromethyl)-3,6-dihydropyrimidin-1(2H)-yl]-5-fluoro-2-(2-fluorophenoxy)benzonitrile, and
2-(2-Chlorophenoxy)-4-[2,6-dioxo-4-(trifluoromethyl)-3,6-dihydropyrimidin-1(2H)-yl]-5-fluorobenzonitrile.

In a further embodiment of the first aspect, the present invention covers compounds of formula (I), supra, in which:

R¹ represents a group selected from:

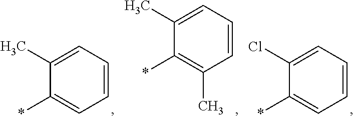

in which “*” represents the point of attachment to the rest of the molecule, and stereoisomers, tautomers, N-oxides, hydrates, solvates, and salts thereof, and mixtures of same,

with the proviso that said compounds of general formula (I) are not:
2-(2-Chlorophenoxy)-4-[2,6-dioxo-4-(trifluoromethyl)-3,6-dihydropyrimidin-1(2H)-yl]-5-fluorobenzonitrile.

In a further embodiment of the first aspect, the present invention covers compounds of formula (I), supra, in which:

R¹ represents a group selected from:

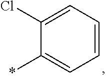 [SMILES: *c1ccccc1Cl]

in which “*” represents the point of attachment to the rest of the molecule, and stereoisomers, tautomers, N-oxides, hydrates, solvates, and salts thereof, and mixtures of same,

with the proviso that said compounds of general formula (I) are not:
2-(2-Chlorophenoxy)-4-[2,6-dioxo-4-(trifluoromethyl)-3,6-dihydropyrimidin-1(2H)-yl]-5-fluorobenzonitrile.

In a further embodiment of the first aspect, the present invention covers compounds of formula (I), supra, in which:

R¹ represents a group selected from:

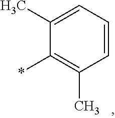 [SMILES: *c1c(C)cccc1C]

in which “*” represents the point of attachment to the rest of the molecule, and stereoisomers, tautomers, N-oxides, hydrates, solvates, and salts thereof, and mixtures of same.

In a further embodiment of the first aspect, the present invention covers compounds of formula (I), supra, in which:

R¹ represents a group selected from:

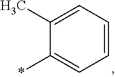 [SMILES: *c1ccccc1C]

in which “*” represents the point of attachment to the rest of the molecule, and stereoisomers, tautomers, N-oxides, hydrates, solvates, and salts thereof, and mixtures of same.

In a further embodiment of the first aspect, the present invention covers compounds of formula (I), supra, in which:

R² represents a chlorine atom,

and stereoisomers, tautomers, N-oxides, hydrates, solvates, and salts thereof, and mixtures of same.

In a further embodiment of the first aspect, the present invention covers compounds of formula (I), supra, in which:

R² represents a cyano group,

and stereoisomers, tautomers, N-oxides, hydrates, solvates, and salts thereof, and mixtures of same,

with the proviso that said compounds of general formula (I) are not:
4-[2,6-Dioxo-4-(trifluoromethyl)-3,6-dihydropyrimidin-1(2H)-yl]-5-fluoro-2-(2-fluorophenoxy)benzonitrile,
2-(2,4-Difluorophenoxy)-4-[2,6-dioxo-4-(trifluoromethyl)-3,6-dihydropyrimidin-1(2H)-yl]-5-fluorobenzonitrile, and
2-(2-Chlorophenoxy)-4-[2,6-dioxo-4-(trifluoromethyl)-3,6-dihydropyrimidin-1(2H)-yl]-5-fluorobenzonitrile.

In a further embodiment of the first aspect, the present invention covers compounds of formula (I), supra, in which:

X represents a group CH,

and stereoisomers, tautomers, N-oxides, hydrates, solvates, and salts thereof, and mixtures of same,

with the proviso that said compounds of general formula (I) are not:
4-[2,6-Dioxo-4-(trifluoromethyl)-3,6-dihydropyrimidin-1(2H)-yl]-5-fluoro-2-(2-fluorophenoxy)benzonitrile,
2-(2,4-Difluorophenoxy)-4-[2,6-dioxo-4-(trifluoromethyl)-3,6-dihydropyrimidin-1(2H)-yl]-5-fluorobenzonitrile, and
2-(2-Chlorophenoxy)-4-[2,6-dioxo-4-(trifluoromethyl)-3,6-dihydropyrimidin-1(2H)-yl]-5-fluorobenzonitrile.

In a further embodiment of the first aspect, the present invention covers compounds of formula (I), supra, in which:

X represents a nitrogen atom N,

and stereoisomers, tautomers, N-oxides, hydrates, solvates, and salts thereof, and mixtures of same.

In a further embodiment of the first aspect, the present invention covers compounds of formula (I), supra, in which:

R⁴ represents a hydrogen atom, a chlorine atom, a bromine atom, an iodine atom, or a group selected from hydroxy, C₁-C₃-alkyl, C₁-C₃-alkoxy and C₁—O₃-haloalkoxy, and stereoisomers, tautomers, N-oxides, hydrates, solvates, and salts thereof, and mixtures of same.

In a further embodiment of the first aspect, the present invention covers compounds of formula (I), supra, in which:

R⁴ represents a hydrogen atom,

and stereoisomers, tautomers, N-oxides, hydrates, solvates, and salts thereof, and mixtures of same.

In a further embodiment of the first aspect, the present invention covers compounds of formula (I), supra, in which:

R⁴ represents a halogen atom,

and stereoisomers, tautomers, N-oxides, hydrates, solvates, and salts thereof, and mixtures of same,

with the proviso that said compounds of general formula (I) are not:
4-[2,6-Dioxo-4-(trifluoromethyl)-3,6-dihydropyrimidin-1(2H)-yl]-5-fluoro-2-(2-fluorophenoxy)benzonitrile,
2-(2,4-Difluorophenoxy)-4-[2,6-dioxo-4-(trifluoromethyl)-3,6-dihydropyrimidin-1(2H)-yl]-5-fluorobenzonitrile, and
2-(2-Chlorophenoxy)-4-[2,6-dioxo-4-(trifluoromethyl)-3,6-dihydropyrinnidin-1(2H)-yl]-5-fluorobenzonitrile.

In a further embodiment of the first aspect, the present invention covers compounds of formula (I), supra, in which:

R⁴ represents a fluorine atom,

and stereoisomers, tautomers, N-oxides, hydrates, solvates, and salts thereof, and mixtures of same,

with the proviso that said compounds of general formula (I) are not:
4-[2,6-Dioxo-4-(trifluoromethyl)-3,6-dihydropyrimidin-1(2H)-yl]-5-fluoro-2-(2-fluorophenoxy)benzonitrile,
2-(2,4-Difluorophenoxy)-4-[2,6-dioxo-4-(trifluoromethyl)-3,6-dihydropyrimidin-1(2H)-yl]-5-fluorobenzonitrile, and
2-(2-Chlorophenoxy)-4-[2,6-dioxo-4-(trifluoromethyl)-3,6-dihydropyrimidin-1(2H)-yl]-5-fluorobenzonitrile.

In a further embodiment of the first aspect, the present invention covers compounds of formula (I), supra, in which:

R⁴ represents a chlorine atom, a bromine atom or an iodine atom,

and stereoisomers, tautomers, N-oxides, hydrates, solvates, and salts thereof, and mixtures of same.

In a further embodiment of the first aspect, the present invention covers compounds of formula (I), supra, in which:

R⁴ represents a group selected from hydroxy, C₁-C₃-alkyl, C₁-C₃-alkoxy and C₁-C₃-haloalkoxy, and stereoisomers, tautomers, N-oxides, hydrates, solvates, and salts thereof, and mixtures of same.

In a further embodiment of the first aspect, the present invention covers compounds of formula (I), supra, in which:

R⁴ represents a C₁-C₃-alkyl group,

and stereoisomers, tautomers, N-oxides, hydrates, solvates, and salts thereof, and mixtures of same.

In a further embodiment of the first aspect, the present invention covers compounds of formula (I), supra, in which:

R⁴ represents a group selected from C₁-C₃-alkoxy and C₁-C₃-haloalkoxy,

and stereoisomers, tautomers, N-oxides, hydrates, solvates, and salts thereof, and mixtures of same.

In a further embodiment of the first aspect, the present invention covers compounds of formula (I), supra, in which:

R⁴ represents a C₁-C₃-alkoxy group,

and stereoisomers, tautomers, N-oxides, hydrates, solvates, and salts thereof, and mixtures of same.

In a further embodiment of the first aspect, the present invention covers compounds of formula (I), supra, in which:

R⁴ represents a methyl group,

and stereoisomers, tautomers, N-oxides, hydrates, solvates, and salts thereof, and mixtures of same.

In a further embodiment of the first aspect, the present invention covers compounds of formula (I), supra, in which:

R⁴ represents a group selected from methoxy, n-propoxy and trifluoromethoxy,

and stereoisomers, tautomers, N-oxides, hydrates, solvates, and salts thereof, and mixtures of same.

In a further embodiment of the first aspect, the present invention covers compounds of formula (I), supra, in which:

R⁴ represents a methoxy group,

and stereoisomers, tautomers, N-oxides, hydrates, solvates, and salts thereof, and mixtures of same.

In a further embodiment of the first aspect, the present invention covers compounds of formula (I), supra, in which:

X represents a group CR³,

and R³ and R⁴ together form a group —CH═CH—CH═CH—,

and stereoisomers, tautomers, N-oxides, hydrates, solvates, and salts thereof, and mixtures of same.

In a further embodiment of the first aspect, the present invention covers compounds of formula (I), supra, in which:

R⁵ represents a bromine atom or a group selected from difluoromethyl, (phenyl)-difluoromethyl, chlorodifluoromethyl, trifluoromethyl, cyclopropyl, methanesulfonyl and benzenesulfonyl,

and stereoisomers, tautomers, N-oxides, hydrates, solvates, and salts thereof, and mixtures of same,

with the proviso that said compounds of general formula (I) are not:
4-[2,6-Dioxo-4-(trifluoromethyl)-3,6-dihydropyrimidin-1(2H)-yl]-5-fluoro-2-(2-fluorophenoxy)benzonitrile,
2-(2,4-Difluorophenoxy)-4-[2,6-dioxo-4-(trifluoromethyl)-3,6-dihydropyrimidin-1(2H)-yl]-5-fluorobenzonitrile, and
2-(2-Chlorophenoxy)-4-[2,6-dioxo-4-(trifluoromethyl)-3,6-dihydropyrimidin-1(2H)-yl]-5-fluorobenzonitrile.

In a further embodiment of the first aspect, the present invention covers compounds of formula (I), supra, in which:

R⁵ represents a bromine atom or a group selected from difluoromethyl, (phenyl)-difluoromethyl, chlorodifluoromethyl, cyclopropyl, methanesulfonyl and benzenesulfonyl,

and stereoisomers, tautomers, N-oxides, hydrates, solvates, and salts thereof, and mixtures of same.

In a further embodiment of the first aspect, the present invention covers compounds of formula (I), supra, in which:
R⁵ represents a halogen atom or a group selected from C₁-C₂-haloalkyl and —S(═O)₂R¹⁰,
   said C₁-C₂-haloalkyl group being optionally substituted with one phenyl group, and in which R¹⁰ represents a group selected from methyl and phenyl,

and stereoisomers, tautomers, N-oxides, hydrates, solvates, and salts thereof, and mixtures of same,

with the proviso that said compounds of general formula (I) are not:
4-[2,6-Dioxo-4-(trifluoromethyl)-3,6-dihydropyrimidin-1(2H)-yl]-5-fluoro-2-(2-fluorophenoxy)benzonitrile,
2-(2,4-Difluorophenoxy)-4-[2,6-dioxo-4-(trifluoromethyl)-3,6-dihydropyrimidin-1(2H)-yl]-5-fluorobenzonitrile, and
2-(2-Chlorophenoxy)-4-[2,6-dioxo-4-(trifluoromethyl)-3,6-dihydropyrinnidin-1(2H)-yl]-5-fluorobenzonitrile.

In a further embodiment of the first aspect, the present invention covers compounds of formula (I), supra, in which:

R⁵ represents a group selected from difluoromethyl, (phenyl)-difluoromethyl, chlorodifluoromethyl, trifluoromethyl and benzenesulfonyl,

and stereoisomers, tautomers, N-oxides, hydrates, solvates, and salts thereof, and mixtures of same,

with the proviso that said compounds of general formula (I) are not:
4-[2,6-Dioxo-4-(trifluoromethyl)-3,6-dihydropyrimidin-1(2H)-yl]-5-fluoro-2-(2-fluorophenoxy)benzonitrile,
2-(2,4-Difluorophenoxy)-4-[2,6-dioxo-4-(trifluoromethyl)-3,6-dihydropyrimidin-1(2H)-yl]-5-fluorobenzonitrile, and
2-(2-Chlorophenoxy)-4-[2,6-dioxo-4-(trifluoromethyl)-3,6-dihydropyrimidin-1(2H)-yl]-5-fluorobenzonitrile.

In a further embodiment of the first aspect, the present invention covers compounds of formula (I), supra, in which:

R⁵ represents a group selected from trifluoromethyl and benzenesulfonyl,

and stereoisomers, tautomers, N-oxides, hydrates, solvates, and salts thereof, and mixtures of same,

with the proviso that said compounds of general formula (I) are not:
4-[2,6-Dioxo-4-(trifluoromethyl)-3,6-dihydropyrimidin-1(2H)-yl]-5-fluoro-2-(2-fluorophenoxy)benzonitrile,
2-(2,4-Difluorophenoxy)-4-[2,6-dioxo-4-(trifluoromethyl)-3,6-dihydropyrimidin-1(2H)-yl]-5-fluorobenzonitrile, and
2-(2-Chlorophenoxy)-4-[2,6-dioxo-4-(trifluoromethyl)-3,6-dihydropyrimidin-1(2H)-yl]-5-fluorobenzonitrile.

In a further embodiment of the first aspect, the present invention covers compounds of formula (I), supra, in which:

R⁵ represents a trifluoromethyl group,

and stereoisomers, tautomers, N-oxides, hydrates, solvates, and salts thereof, and mixtures of same,

with the proviso that said compounds of general formula (I) are not:
4-[2,6-Dioxo-4-(trifluoromethyl)-3,6-dihydropyrimidin-1(2H)-yl]-5-fluoro-2-(2-fluorophenoxy)benzonitrile,
2-(2,4-Difluorophenoxy)-4-[2,6-dioxo-4-(trifluoromethyl)-3,6-dihydropyrimidin-1(2H)-yl]-5-fluorobenzonitrile, and
2-(2-Chlorophenoxy)-4-[2,6-dioxo-4-(trifluoromethyl)-3,6-dihydropyrimidin-1(2H)-yl]-5-fluorobenzonitrile.

In a further embodiment of the first aspect, the present invention covers compounds of formula (I), supra, in which:

R⁵ represents a benzenesulfonyl group,

and stereoisomers, tautomers, N-oxides, hydrates, solvates, and salts thereof, and mixtures of same.

In a particular further embodiment of the first aspect, the present invention covers combinations of two or more of the above mentioned embodiments under the heading “further embodiments of the first aspect of the present invention”.

The present invention covers any sub-combination within any embodiment or aspect of the present invention of compounds of general formula (I), supra.

The present invention covers any sub-combination within any embodiment or aspect of the present invention of intermediate compounds of general formulae (III), (VI, and (IX), see below.

The present invention covers the compounds of general formula (I) which are disclosed in the Example Section of this text, infra.

General Synthesis of the Compounds According the Invention

The compounds according to the invention of general formula (I) can be prepared according to the following schemes 2, 3, 4 and 5. The schemes and procedures described below illustrate synthetic routes to the compounds of general formula (I) of the invention and are not intended to be limiting. It is clear to the person skilled in the art that the order of transformations as exemplified in schemes 2, 3, 4 and 5 can be modified in various ways. The order of transformations exemplified in these schemes is therefore not intended to be limiting. In addition, modification of any of the substituents, R¹, R², R³, R⁴ or R⁵ can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs and in the Experimental Section.

Compounds of formula (I), and the intermediates used in their synthesis, may be chiral, e.g. as a result of axial chirality as discussed supra, or if at least one of the substituents R¹, R², R³, R⁴ and R⁵ features an asymmetric carbon or sulfur atom, and may then be formed as mixtures of stereoisomers, such as atropisomers. Said mixtures of stereoisomers can be separated by methods well known the person skilled in the art, such as preparative chromatography, such as high pressure liquid chromatography (HPLC) or superfluid chromatography (SFC) using chiral stationary phases, which are commercially available in considerable variety.

Four routes for the preparation of compounds of general formula (I) are described in Schemes 2, 3, 4 and 5.

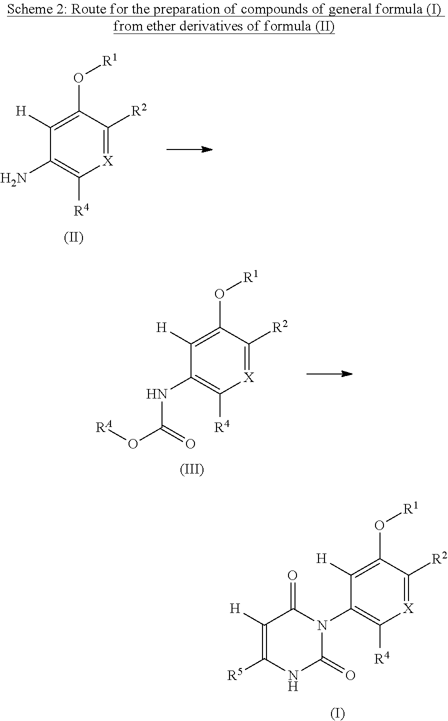

Compounds of the general formula (I) can be obtained from aromatic ether derivatives of formula (II), in which R¹, R², R⁴ and X have the meaning as given for general formula (I), by reaction of the amino group therein with reagents of the formula Cl—C(═O)—OR^{A}, in which R^{A} represents a C₁-C₄-alkyl group, preferably methyl or ethyl, in pyridine as a solvent, at a temperature in the range from −20° C. to room temperature, preferably at 0° C., for a time in the range from 15 minutes to 6 hours, preferably 2 hours, to give intermediate carbamates of formula (III). Said carbamates of formula (III) can subsequently be reacted with a mixture, previously prepared from sodium hydride and an acrylate ester of the formula R⁵—C(NH₂)═C(H)—C(═O)—OR^{A′}, in which R⁵ has the meaning as given for general formula (I) and in which R^{A′} represents a C₁-C₄-alkyl group, preferably methyl or ethyl, in a dipolar aprotic solvent such as dimethylsulfoxide, N,N-dimethylformamide, N,N-dimethylacetamide or 1-methyl-2-pyrrolidinone, preferably N,N-dimethylformamide, at a temperature in the range from −20° C. to room temperature, preferably at 0° C., for a time in the range from 30 minutes to 1 hour, preferably 40 minutes, carbamates of formula (III) being then reacted with such mixture at a temperature in the range from 60° C. to 120° C., preferably 90° C., for a time in a range from 12 hours to 24 hours, preferably 18-20 hours, to give compounds of the general formula (I). Specific examples are described in the Experimental Section.

Reagents of the formula Cl—C(═O)—OR^{A} and acrylate esters of the formula R⁵—C(NH₂)═C(H)—C(═O)—OR^{A′} are well known to the person skilled in the art, and many of them are also commercially available. Aromatic ether derivatives of formula (II) are also known to the person skilled in the art, and can be readily be prepared e.g. from the corresponding fluoroarenes of formula (Vila) by means of nucleophilic aromatic substitution with a phenolate anion R¹O⁻, or by subjecting bromoarenes of formula (IVa) to a coupling reaction with phenols R¹OH, in the presence of copper (l) chloride, cesium carbonate as a base, and commercially available 2,2,6,6-tetramethylheptane-2,5-dione, in a solvent such as 1-methyl-2-pyrrolidinone, as shown in Scheme 2a below. Specific examples are described in the Experimental Section.

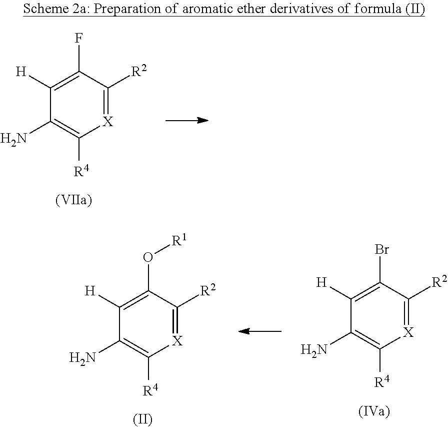

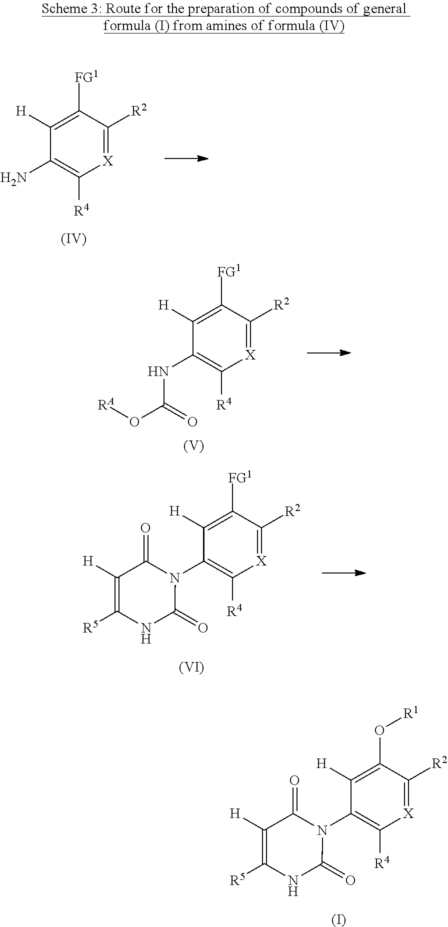

Alternatively, compounds of the general formula (I) can be obtained from amines of formula (IV), in which R², R⁴ and X have the meaning as given for general formula (I), and in which FG¹ represents a chlorine, a bromine or an iodine atom, preferably a bromine atom, by reaction of the amino group therein with reagents of the formula Cl—C(═O)—OR^{A}, in which R^{A} represents a C₁-C₄-alkyl group, preferably methyl or ethyl, in pyridine as a solvent, at a temperature in the range from −20° C. to room temperature, preferably at 0° C., for a time in the range from 15 minutes to 6 hours, preferably from 30 minutes to 2 hours, to give intermediate carbamates of formula (V)

Said carbamates of formula (V) can subsequently be reacted with a mixture, previously prepared from sodium hydride and an acrylate ester of the formula R⁵—C(NH₂)═C(H)—C(═O)—OR^{A′}, in which R⁵ has the meaning as given for general formula (I) and in which R^{A′} represents a C₁-C₄-alkyl group, preferably methyl or ethyl, in a dipolar aprotic solvent such as dimethylsulfoxide, N,N-dimethylformamide, N,N-dimethylacetamide or N-methylpyrrolidin-2-one, preferably N,N-dimethylformamide, at a temperature in the range from −20° C. to room temperature, preferably at 0° C., for a time in the range from 30 minutes to 1 hour, preferably 40 minutes, carbamates of formula (V) being then reacted with such mixture at a temperature in the range from 60° C. to 120° C., preferably 90° C., for a time ranging from 12 hours to 24 hours, preferably 18-20 hours, to give intermediate compounds of formula (VI).

Intermediate compounds of formula (VI), in turn, can be reacted with phenols of the formula R¹—OH, in which R¹ has the meaning as given for general formula (I), in the presence of N,N-dimethylglycine, copper(I) iodide, and a base, such as an alkali carbonate or an alkali phosphate, preferably cesium carbonate, in a dipolar aprotic solvent selected from N, N-dimethylformamide, N,N-dimethylacetamide or N-methylpyrrolidin-2-one, preferably N,N-dimethylformamide, at a temperature in the range from 100° C. to 150° C., preferably 140° C., for a time ranging from 12 hours to 24 hours, preferably 18-20 hours, to give compounds of the general formula (I). Specific examples are described in the Experimental Section.

Said amines of formula (IV), phenols of the formula R¹—OH, N,N-dimethylglycine, copper(I) iodide, and bases such as alkali carbonates or alkali phosphates, including cesium carbonate, are well-known to the person skilled in the art and are commercially available.

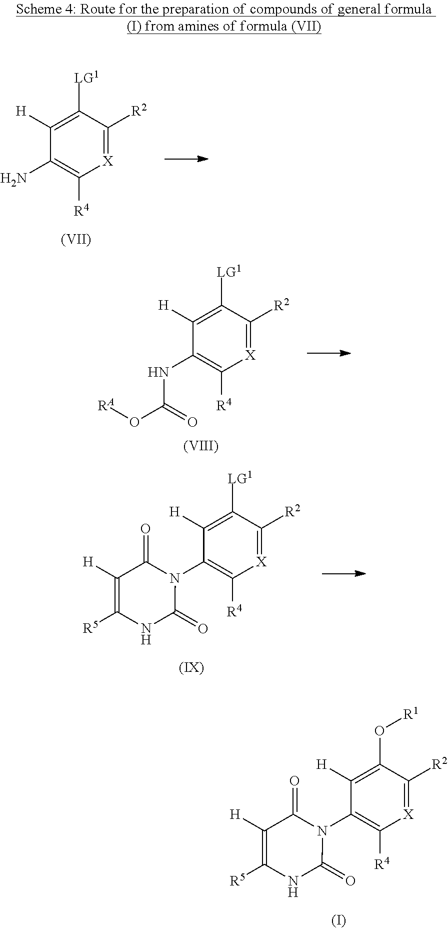

Further even, compounds of the general formula (I) can be obtained from amines of formula (VII), in which R², R⁴ and X have the meaning as given for general formula (I), and in which LG′ represents a chlorine atom or a fluorine atom, preferably a fluorine atom, by reaction of the amino group therein with reagents of the formula Cl—C(═O)—OR^{A}, in which R^{A} represents a C₁-C₄-alkyl group, preferably methyl or ethyl, in pyridine as a solvent, at a temperature in the range from −20° C. to room temperature, preferably at 0° C., for a time in the range from 15 minutes to 6 hours, preferably from 30 minutes to 2 hours, to give intermediate carbamates of formula (VIII).

Said carbamates of formula (VIII) can subsequently be reacted with a mixture, previously prepared from sodium hydride and an acrylate ester of the formula R⁵—C(NH₂)═C(H)—C(═O)—OR^{A′}, in which R⁵ has the meaning as given for general formula (I) and in which R^{A′} represents a C₁—O₄-alkyl group, preferably methyl or ethyl, in a dipolar aprotic solvent such as dimethylsulfoxide, N,N-dimethylformamide, N,N-dimethylacetamide or N-methylpyrrolidin-2-one, preferably N,N-dimethylformamide, at a temperature in the range from −20° C. to room temperature, preferably at 0° C., for a time in the range from 30 minutes to 1 hour, preferably 40 minutes, carbamates of formula (V) being then reacted with such mixture at a temperature in the range from 60° C. to 120° C., preferably 90° C., for a time ranging from 12 hours to 24 hours, preferably 18-20 hours, to give intermediate compounds of formula (IX).

Intermediate compounds of formula (IX), in turn, can be reacted with phenols of the formula R¹—OH, in which R¹ has the meaning as given for general formula (I), in the presence of a base, such as an alkali carbonate or an alkali phosphate, preferably potassium carbonate, in a dipolar aprotic solvent such as dimethylsulfoxide, N,N-dimethylformamide, N,N-dimethylacetamide or N-methylpyrrolidin-2-one, preferably dimethylsulfoxide, at a temperature in the range from 80° C. to 140° C., preferably 110° C., for a time ranging from 12 hours to 24 hours, preferably 20 hours, to give compounds of the general formula (I). Specific examples are described in the Experimental Section.

Said amines of formula (VII) are well-known to the person skilled in the art and many of them are also commercially available.

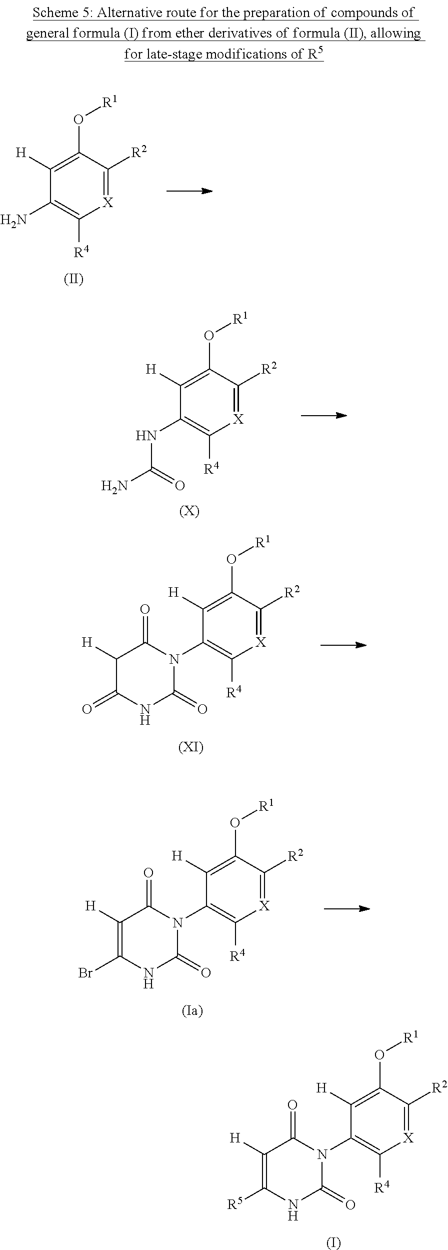

In a further alternative approach, particularly suitable for certain late-stage modifications of R⁵, compounds of the general formula (I) can be obtained from aromatic ether derivatives of formula (II), in which R¹, R², R⁴ and X have the meaning as given for general formula (I), by reaction of the amino group therein with commercially available chlorosulfonyl-isocyanate (CAS: 1189-71-5) in acetonitrile as a solvent, at a temperature in the range from 0° C. to 50° C., preferably at 25° C., for a time in the range from 15 minutes to 2 hours, preferably 0.5 hours, to give intermediate ureas of formula (X). Said ureas of formula (X) can be further converted to barbituric acid derivatives of formula (XI) by reacting with a commercially available malonic ester R^{A}O—C(═O)—CH₂—C(═O)—OR^{A}, in the presence of an alkali alkoxide M⁺[OR^{A}]⁻, in an aliphatic alcohol of the formula R^{A}—OH as a solvent, in which R^{A}, independently in each occurrence, represents a C₁-C₄-alkyl group, preferably methyl or ethyl, and in which M⁺ represents an alkali metal cation, preferably a sodium (Na⁺) or potassium (K⁺) cation, at a temperature in a range from 60° C. to 120° C., preferably in a range from 80° C. to 110° C., more preferably at 100° C., for a time in the range from 2 hours to 12 hours, preferably 6 hours. Said barbituric acid derivatives of formula (XI) can be reacted with phosphoryl bromide (POBr₃), in acetonitrile as a solvent, at a temperature in a range from 90° C. to 130° C., preferably at 110° C., for a time in the range from 15 minutes to 2 hours, preferably 0.5 hours, to give compounds of formula (Ia), representing a sub-set of general formula (I) in which R⁵ represents a bromine atom. Said compounds of formula (Ia) can, in turn, be converted into compounds of general formula (I) featuring groups R⁵ which are different from a bromine atom, e.g. by the well-known Suzuki coupling with, for example, optionally substituted phenylboronic acids (or esters thereof), to give compounds of the general formula (I) in which R⁵ represents an optionally substituted phenyl group, as defined for general formula (I), or by reacting said compounds of formula (Ia) with sulfinate salts of the formula R¹⁰SO₂⁻M⁺, in which R¹⁰ has the meaning as given for general formula (I), and in which M⁺ represents an alkali metal cation, preferably a sodium (Na⁺) or potassium (K⁺) cation, in the presence of catalytic amounts of copper (I) trifluoromethane sulfonate benzene complex, in the presence of the ligand (+/−)-trans-1,2-diaminocyclohexane, in dimethylsulfoxide as a solvent, at a temperature in a range from 90° C. to 130° C., preferably at 110° C., for a time in the range from 12 hours to 50 hours, preferably 20 hours, to give compounds of formula (I) in which R⁵ represents a group —S(═O)₂R¹⁰. Specific examples are described in the Experimental Section.

In accordance with a second aspect, the present invention covers methods of preparing compounds of general formula (I) as defined supra, said methods comprising the step of allowing an intermediate compound of general formula (III):

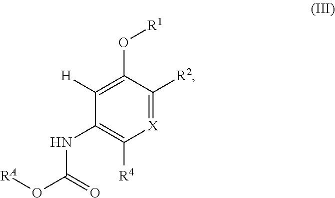

in which R¹, R², R⁴ and X are as defined for the compound of general formula (I) as defined supra, and in which R^{A} represents a C₁-C₄-alkyl group,

to react with a mixture, previously prepared from sodium hydride and an acrylate ester of the formula R⁵—C(NH₂)═C(H)—C(═O)—OR^{A′} in a dipolar aprotic solvent, in which R⁵ is as defined for the compound of general formula (I), and in which R^{A′} represents a C₁-C₄-alkyl group,

thereby giving a compound of general formula (I):

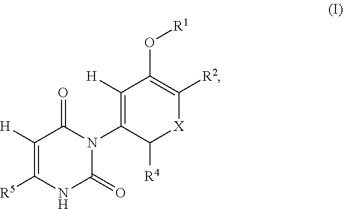

in which R¹, R², R⁴, R⁵ and X are as defined supra.

In a further embodiment of the second aspect, the present invention covers methods of preparing compounds of general formula (I) as defined supra, said methods comprising the step of allowing an intermediate compound of general formula (VI):

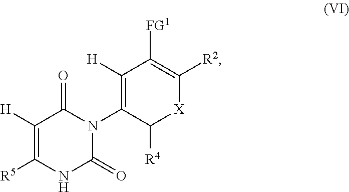

in which R², R⁴, R⁵ and X are as defined for the compound of general formula (I) as defined supra, and in which FG¹ represents a chlorine, a bromine or an iodine atom,

to react with a phenol of the formula R¹—OH, in which R¹ has the meaning as given for general formula (I), in the presence of N,N-dimethylglycine and copper(I) iodide,

thereby giving a compound of general formula (I):

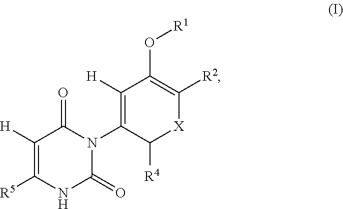

in which R¹, R², R⁴, R⁵ and X are as defined supra.

In a further embodiment of the second aspect, the present invention covers methods of preparing compounds of general formula (I) as defined supra, said methods comprising the step of allowing an intermediate compound of general formula (IX):

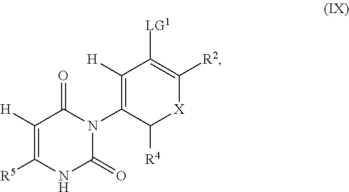

in which R², R⁴, R⁵ and X are as defined for the compound of general formula (I) as defined supra, and in which LG¹ represents a chlorine atom or a fluorine atom,

to react with a phenol of the formula R¹—OH, in which R¹ has the meaning as given for general formula (I), in the presence of a base,

thereby giving a compound of general formula (I):

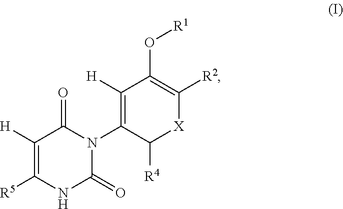

in which R¹, R², R⁴, R⁵ and X are as defined supra.

In accordance with a third aspect, the present invention covers methods of preparing compounds of general formula (I) as defined supra, said methods comprising the step of allowing an intermediate compound of general formula (III):

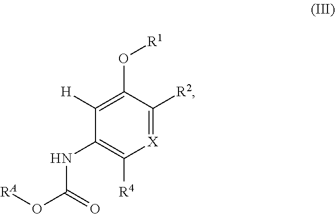

in which R¹, R², R⁴ and X are as defined for the compound of general formula (I) as defined supra, and in which R^{A} represents a C₁-C₄-alkyl group,

to react with a mixture, previously prepared from sodium hydride and an acrylate ester of the formula R⁵—C(NH₂)═C(H)—C(═O)—OR^{A′} in a dipolar aprotic solvent, in which R⁵ is as defined for the compound of general formula (I), and in which R^{A′} represents a C₁-C₄-alkyl group,

thereby giving a compound of general formula (I):

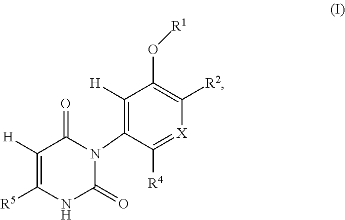

in which R¹, R², R⁴, R⁵ and X are as defined supra,

then optionally converting said compound into solvates, salts and/or solvates of such salts using the corresponding (i) solvents and/or (ii) bases or acids.

In a further embodiment of the third aspect, the present invention covers methods of preparing compounds of general formula (I) as defined supra, said methods comprising the step of allowing an intermediate compound of general formula (VI):

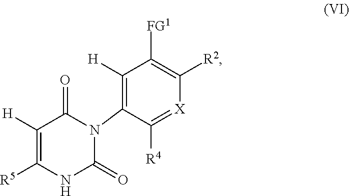

in which R², R⁴, R⁵ and X are as defined for the compound of general formula (I) as defined supra, and in which FG¹ represents a chlorine, a bromine or an iodine atom,

to react with a phenol of the formula R¹—OH, in which R¹ has the meaning as given for general formula (I), in the presence of N,N-dimethylglycine and copper(I) iodide,

thereby giving a compound of general formula (I):

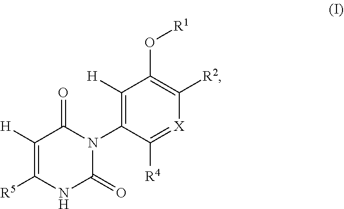

in which R¹, R², R⁴, R⁵ and X are as defined supra,

then optionally converting said compound into solvates, salts and/or solvates of such salts using the corresponding (i) solvents and/or (ii) bases or acids.

In a further embodiment of the third aspect, the present invention covers methods of preparing compounds of general formula (I) as defined supra, said methods comprising the step of allowing an intermediate compound of general formula (IX):

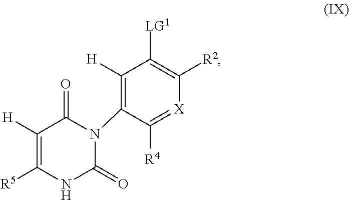

in which R², R⁴, R⁵ and X are as defined for the compound of general formula (I) as defined supra, and in which LG¹ represents a chlorine atom or a fluorine atom,

to react with a phenol of the formula R¹—OH, in which R¹ has the meaning as given for general formula (I), in the presence of a base,

thereby giving a compound of general formula (I):

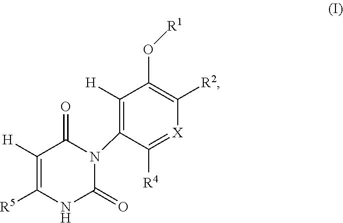

in which R¹, R², R⁴, R⁵ and X are as defined supra,

then optionally converting said compound into solvates, salts and/or solvates of such salts using the corresponding (i) solvents and/or (ii) bases or acids.

The present invention covers methods of preparing compounds of the present invention of general formula (I), said methods comprising the steps as described in the Experimental Section herein.

In accordance with a fourth aspect, the present invention covers intermediate compounds which are useful for the preparation of the compounds of general formula (I), supra.

Particularly, the invention covers the intermediate compounds of general formula (III):

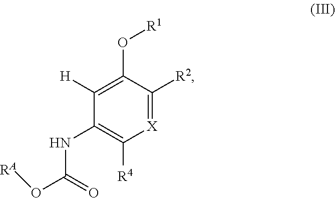

in which R¹, R², R⁴ and X are as defined for the compound of general formula (I) as defined supra, and in which R^{A} represents a C₁-C₄-alkyl group.

In another embodiment of the fourth aspect, the present invention covers the intermediate compounds of general formula (VI):

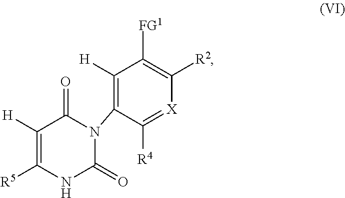

in which R², R⁴, R⁵ and X are as defined for the compound of general formula (I) as defined supra, and in which FG¹ represents a chlorine, a bromine or an iodine atom.

In another embodiment of the fourth aspect, the present invention covers the intermediate compounds of general formula (IX):

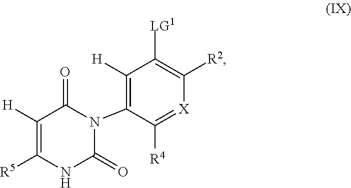

in which R², R⁴, R⁵ and X are as defined for the compound of general formula (I) as defined supra, and in which LG¹ represents a chlorine atom or a fluorine atom.

In accordance with a fifth aspect, the present invention covers the use of said intermediate compounds for the preparation of a compound of general formula (I) as defined supra.

Particularly, the inventions covers the use of intermediate compounds of general formula (III):

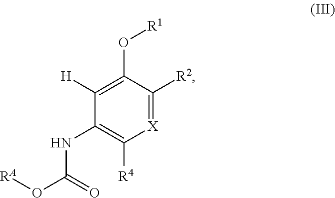

in which R¹, R², R⁴ and X are as defined for the compound of general formula (I) as defined supra, and in which R^{A} represents a C₁-C₄-alkyl group, for the preparation of a compound of general formula (I) as defined supra.

In another embodiment of the fifth aspect, the present invention covers the use of said intermediate compounds of general formula (VI):

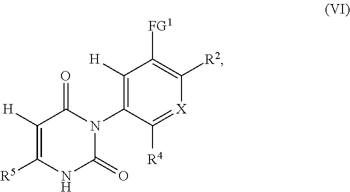

in which R², R⁴, R⁵ and X are as defined for the compound of general formula (I) as defined supra, and in which FG¹ represents a chlorine, a bromine or an iodine atom, for the preparation of a compound of general formula (I) as defined supra.

In another embodiment of the fifth aspect, the present invention covers the use of said intermediate compounds of general formula (IX):

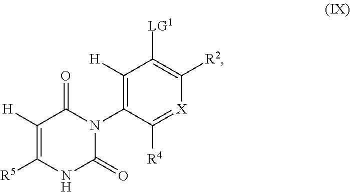

in which R², R⁴, R⁵ and X are as defined for the compound of general formula (I) as defined supra, and in which LG¹ represents a chlorine atom or a fluorine atom, for the preparation of a compound of general formula (I) as defined supra.

The present invention covers the intermediate compounds which are disclosed in the Experimental Section of this text, infra.

The present invention covers any sub-combination within any embodiment or aspect of the present invention of intermediate compounds of general formulae (III), (VI, and (IX), supra.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

Compounds of general formula (I) of the present invention demonstrate a valuable pharmacological spectrum of action which could not have been predicted. Compounds of the present invention have surprisingly been found to effectively inhibit BCAT 1 and it is possible therefore that said compounds be used for the treatment or prophylaxis of diseases, such as hyperproliferative disorders, preferably cancer in humans and animals.

Compounds of the present invention can be utilized to inhibit, block, reduce, decrease, etc., cell proliferation and/or cell division, and/or produce apoptosis, and for the treatment of cancer induced cachexia. This method comprises administering to a mammal in need thereof, including a human, an amount of a compound of general formula (I) of the present invention, or a N-oxide, a pharmaceutically acceptable salt, isomer, polymorph, metabolite, hydrate, solvate or ester thereof, which is effective to treat the disorder.

Hyperproliferative disorders include, but are not limited to, for example: fibrosis, solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, invasive ductal carcinoma, invasive lobular carcinoma, ductal carcinoma in situ, lobular carcinoma in situ, and also antiestrogen-resistant and ERalpha-negative breast cancer.

Examples of cancers of the respiratory tract include, but are not limited to, small-cell lung carcinoma (SCLC) and non-small-cell lung carcinoma (NSCLC), as well as bronchial adenoma and pleuropulmonary blastoma.

Examples of brain cancers include, but are not limited to gliomas, such as brain stem and hypophtalmic glioma, cerebellar and cerebral astrocytoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urothelial and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, for example the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemotherapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.
9. provide an improvement of quality of life in patients with BCAT1 expression related diseases

In addition, the compounds of general formula (I) of the present invention can also be used in combination with radiotherapy and/or surgical intervention.

In a further embodiment of the present invention, the compounds of general formula (I) of the present invention may be used to sensitize a cell to radiation, i.e. treatment of a cell with a compound of the present invention prior to radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of general formula (I) of the present invention prior to the treatment of the cell to cause or induce cell death. In one aspect, after the cell is treated with one or more compounds of general formula (I) of the present invention, the cell is treated with at least one compound, or at least one method, or a combination thereof, in order to cause DNA damage for the purpose of inhibiting the function of the normal cell or killing the cell.

In other embodiments of the present invention, a cell is killed by treating the cell with at least one DNA damaging agent, i.e. after treating a cell with one or more compounds of general formula (I) of the present invention to sensitize the cell to cell death, the cell is treated with at least one DNA damaging agent to kill the cell. DNA damaging agents useful in the present invention include, but are not limited to, chemotherapeutic agents (e.g. cis platin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of general formula (I) of the present invention is administered to a cell prior to the radiation or other induction of DNA damage in the cell. In another aspect of the invention, a compound of general formula (I) of the present invention is administered to a cell concomitantly with the radiation or other induction of DNA damage in the cell. In yet another aspect of the invention, a compound of general formula (I) of the present invention is administered to a cell immediately after radiation or other induction of DNA damage in the cell has begun.

In another aspect, the cell is in vitro. In another embodiment, the cell is in vivo.

Compounds of the present invention can be utilized to inhibit BOAT 1. This method comprises administering to a mammal in need thereof, including a human, an amount of a compound of this invention, or a pharmaceutically acceptable salt, isomer, polymorph, metabolite, hydrate, solvate or ester thereof; which is effective to treat the disorder.

Disorders suitable for treatment or prophylaxis with the compounds of the present invention include but are not limited to cancer, such as glioma, lung cancer, such as non-small-cell lung cancer (NSCLC), breast cancer, such as anti-estrogen resistant breast cancer and ERalpha-negative breast cancer, leukemia, prostate cancer, ovarian cancer, urothelial carcinoma, gastric cancer and nasopharyngeal carcinoma, for example, particularly, glioma and breast cancer, further, cancer-induced cachexia and certain non-oncological diseases such as fibrosis.

The present invention also provides methods of treating cancer, such as glioma, lung cancer, such as non-small-cell lung cancer (NSCLC), breast cancer, such as anti-estrogen resistant breast cancer and ERalpha-negative breast cancer, leukemia, prostate cancer, ovarian cancer, urothelial carcinoma, gastric cancer and nasopharyngeal carcinoma, for example, particularly, of glioma and breast cancer, further, of cancer-induced cachexia and of certain non-oncological diseases such as fibrosis.

These disorders have been well characterized in humans, but also exist with a similar etiology in other mammals, and can be treated by administering pharmaceutical compositions of the present invention.

The term “treating” or “treatment” as used in the present text is used conventionally, e.g., the management or care of a subject for the purpose of combating, alleviating, reducing, relieving, improving the condition of a disease or disorder, such as a cancer, a tumour, or a carcinoma.

The compounds of the present invention can be used in particular in therapy and prevention, i.e. prophylaxis, of cancer, such as glioma, lung cancer, such as non-small-cell lung cancer (NSCLC), breast cancer, such as anti-estrogen resistant breast cancer and ERalpha-negative breast cancer, leukemia, prostate cancer, ovarian cancer, urothelial carcinoma, gastric cancer and nasopharyngeal carcinoma, for example, particularly, of glioma and breast cancer, further, of cancer-induced cachexia and of certain non-oncological diseases such as fibrosis.

In accordance with a further aspect, the present invention covers compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, in particular of cancer, such as glioma, lung cancer, such as non-small-cell lung cancer (NSCLC), breast cancer, such as anti-estrogen resistant breast cancer and ERalpha-negative breast cancer, leukemia, prostate cancer, ovarian cancer, urothelial carcinoma, gastric cancer and nasopharyngeal carcinoma, for example, particularly, of glioma and breast cancer, further, of cancer-induced cachexia and of certain non-oncological diseases such as fibrosis.

The pharmaceutical activity of the compounds according to the invention can be explained by their activity as inhibitors of BOAT 1.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, for the treatment or prophylaxis of diseases, in particular of cancer, such as glioma, lung cancer, such as non-small-cell lung cancer (NSCLC), breast cancer, such as anti-estrogen resistant breast cancer and ERalpha-negative breast cancer, leukemia, prostate cancer, ovarian cancer, urothelial carcinoma, gastric cancer and nasopharyngeal carcinoma, for example, particularly, of glioma and breast cancer, further, of cancer-induced cachexia and of certain non-oncological diseases such as fibrosis.

In accordance with a further aspect, the present invention covers the use of a compound of formula (I), described supra, or a stereoisomer, a tautomer, an N-oxide, a hydrate, a solvate, or a salt thereof, particularly a pharmaceutically acceptable salt thereof, or a mixture of same, for the prophylaxis or treatment of diseases, in particular of cancer, such as glioma, lung cancer, such as non-small-cell lung cancer (NSCLC), breast cancer, such as anti-estrogen resistant breast cancer and ERalpha-negative breast cancer, leukemia, prostate cancer, ovarian cancer, urothelial carcinoma, gastric cancer and nasopharyngeal carcinoma, for example, particularly, of glioma and breast cancer, further, of cancer-induced cachexia and of certain non-oncological diseases such as fibrosis.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of diseases, in particular of cancer, such as glioma, lung cancer, such as non-small-cell lung cancer (NSCLC), breast cancer, such as anti-estrogen resistant breast cancer and ERalpha-negative breast cancer, leukemia, prostate cancer, ovarian cancer, urothelial carcinoma, gastric cancer and nasopharyngeal carcinoma, for example, particularly, of glioma and breast cancer, further, of cancer-induced cachexia and of certain non-oncological diseases such as fibrosis.

In accordance with a further aspect, the present invention covers use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular of cancer, such as glioma, lung cancer, such as non-small-cell lung cancer (NSCLC), breast cancer, such as anti-estrogen resistant breast cancer and ERalpha-negative breast cancer, leukemia, prostate cancer, ovarian cancer, urothelial carcinoma, gastric cancer and nasopharyngeal carcinoma, for example, particularly, of glioma and breast cancer, further, of cancer-induced cachexia and of certain non-oncological diseases such as fibrosis.

In accordance with a further aspect, the present invention covers a method of treatment or prophylaxis of diseases, in particular of cancer, such as glioma, lung cancer, such as non-small-cell lung cancer (NSCLC), breast cancer, such as anti-estrogen resistant breast cancer and ERalpha-negative breast cancer, leukemia, prostate cancer, ovarian cancer, urothelial carcinoma, gastric cancer and nasopharyngeal carcinoma, for example, particularly, of glioma and breast cancer, further, of cancer-induced cachexia and of certain non-oncological diseases such as fibrosis, using an effective amount of a compound of general formula (I), as described supra, or stereoisomers, tautomers, N-oxides, hydrates, solvates, and salts thereof, particularly pharmaceutically acceptable salts thereof, or mixtures of same.

In accordance with a further aspect, the present invention covers pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore covers pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®) lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropyl-cellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropyl-methylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In accordance with another aspect, the present invention covers pharmaceutical combinations, in particular medicaments, comprising at least one compound of general formula (I) of the present invention and at least one or more further active ingredients, in particular for the treatment and/or prophylaxis of particular of cancer, such as glioma, lung cancer, such as non-small-cell lung cancer (NSCLC), breast cancer, such as anti-estrogen resistant breast cancer and ERalpha-negative breast cancer, leukemia, prostate cancer, ovarian cancer, urothelial carcinoma, gastric cancer and nasopharyngeal carcinoma, for example, particularly, of glioma and breast cancer.

Particularly, the present invention covers a pharmaceutical combination, which comprises:
one or more first active ingredients, in particular compounds of general formula (I) as defined supra, and
one or more further active ingredients, in particular of cancer, such as glioma, lung cancer, such as non-small-cell lung cancer (NSCLC), breast cancer, such as anti-estrogen resistant breast cancer and ERalpha-negative breast cancer, leukemia, prostate cancer, ovarian cancer, urothelial carcinoma, gastric cancer and nasopharyngeal carcinoma, for example, particularly, of glioma and breast cancer.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with known agents for the treatment of cancer.

Examples of Known Agents for the Treatment of Cancer Include:

131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

Based upon standard laboratory techniques known to evaluate compounds useful for the treatment of cancer, cancer-induced cachexia and fibrosis, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### EXPERIMENTAL SECTION

NMR peak forms are stated as they appear in the spectra, possible higher order effects have not been considered.

The ¹H-NMR data of selected compounds are listed in the form of ¹H-NMR peaklists. Therein, for each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of the particular target compound, peaks of impurities, ¹³C satellite peaks, and/or spinning sidebands. The peaks of stereoisomers, and/or peaks of impurities are typically displayed with a lower intensity compared to the peaks of the target compound (e.g., with a purity of >90%). Such stereoisomers and/or impurities may be typical for the particular manufacturing process, and therefore their peaks may help to identify a reproduction of the manufacturing process on the basis of “by-product fingerprints”. An expert who calculates the peaks of the target compound by known methods (MestReC, ACD simulation, or by use of empirically evaluated expectation values), can isolate the peaks of the target compound as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. http://www.researchdisclosure.com/searching-disclosures, Research Disclosure Database Number 605005, 2014, 1 Aug. 2014). In the peak picking routine, as described in the Research Disclosure Database Number 605005, the parameter “MinimumHeight” can be adjusted between 1% and 4%. However, depending on the chemical structure and/or depending on the concentration of the measured compound it may be reasonable to set the parameter “MinimumHeight”<1%.

In the text of the protocols, the numbers of the compounds are given in bold. The intermediates are referred to by the prefix “INT-”. Chemical names were generated using the ACD/Name batch software from ACD/Labs, or Autonom 200 in order to follow the IUPAC nomenclature. In some cases generally accepted names of commercially available reagents were used in place of ACD/Name generated names.

The following table 1 lists the abbreviations used in this paragraph and in the Examples section as far as they are not explained within the text body. Other abbreviations have their meanings customary per se to the skilled person.

TABLE 1
Abbreviations

| Abbreviation | Meaning |
|---|---|
| aq. | aqueous |
| DCM | dichloromethane |
| DIPEA | N,N-diisopropylethylamine |
| DMAP | 4-(Dimethylamino)-pyridin |
| DMF | N,N-dimethylformamide |
| DMSO | dimethylsulfoxide |
| eq. | equivalent |
| EtOH | ethanol |
| EtOAc | ethyl acetate |
| h | hour(s) |
| HT | High throughput |
| HPLC | High-performance liquid chromatography |
| NMR | nuclear magnetic resonance spectroscopy: |
| | chemical |
| MeOH | methanol |
| MS | mass spectrometry |
| min | minute(s) |
| MTBE | 2-Methoxy-2-methylpropan |
| NFSI | N-Fluorobenzenesulfonimide |
| PE | petroleum ether |
| PyBOP | Benzotriazol-1-yl- |
| | oxytripyrrolidinophosphonium |
| | hexafluorophosphate |
| RT | room temperature |
| Rt | retention time (as measured either with |
| | HPLC or UPLC) |
| Selectfluor ® | 1-Chloromethyl-4-fluoro-1,4- |
| | diazoniabicyclo[2.2.2]octane |
| | bis(tetrafluoroborate) |
| T3P | 1-Propanephosphonic anhydride |
| TFA | Trifluoroacetic acid |
| THF | tetrahydrofuran |
| TLC | Thin-layer chromatographie |
| TMS | Trimethylsilyl- |
| TPP | Triphenylphospine |

The following table lists the abbreviations used herein.

The various aspects of the invention described in this application are illustrated by the following examples which are not meant to limit the invention in any way.

The example testing experiments described herein serve to illustrate the present invention and the invention is not limited to the examples given.

### Experimental Section—General Part

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be removed by trituration using a suitable solvent or solvent mixture. In some cases, the compounds may be purified by chromatography, particularly flash column chromatography, using for example prepacked silica gel cartridges, e.g. Biotage SNAP cartidges KP-Sil® or KP-NH® in combination with a Biotage autopurifier system (SP4® or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

Analytical Methods

All NMR spectra were recorded on Bruker Avance III HD and Bruker Plus spectrometers. ¹H spectra were obtained at 400 MHz and referenced to residual solvent signals (2.50 ppm for dimethyl sulfoxide, 7.26 ppm for Chloroform, 3.30 ppm for methanol). ¹⁹F chemical shifts were referenced external to C₆F₆ at—162.90 ppm. All spectra were obtained at ambient temperature (22° C.+/−1° C.). Peak forms and multiplicities are specified as apparent in the spectra, potential higher-order effects have not been considered.

Optical rotations were measured on a P2000 JASCO polarimeter using a 1 mL microcell (10 cm thickness, 3 mm diameter) with an alpha-D-line and a Na lamp at 20° C. Solvent and concentration are given in brackets.

Chromatographic Methods

Preparative column chromatographies, in particular, flash column chromatographies were performed using for example prepacked silica gel cartridges, e.g. Biotage SNAP cartridges KP-Sil® or KP-NH® in combination with a Biotage autopurifier system (SP4® or Isolera Four®) and eluents such as gradients of hexane/ethyl acetate or dichloromethane/methanol were used.

Preparative HPLC (HT) were performed on a Waters Autopurification MS SingleQuad instrument using a Waters XBrigde C₁₈ 5 μm 100×30 mm column (flow: 70 mL/min; temperature: 25° C.; DAD scan: 210-400 nm) under either acidic conditions [eluent A: water+0.1 vol % formic acid (99%), eluent B: acetonitrile; gradient: 0-5.5 min 5-100% B] or a basic [eluent A: water+0.2 vol % aqueous ammonia, eluent B: acetonitrile; gradient: 0-5.5 min 5-100% B]. Before injection, the crude products were dissolved in a solvent (most of the time dimethyl sulfoxide, tetrahydrofuran or dichloromethane) and then filtered.

UPLC-MS Standard Procedures

Analytical UPLC-MS was performed as described below. The masses (m/z) are reported from the positive mode electrospray ionisation unless the negative mode is indicated (ESI−). In most of the cases method 1 is used. If not, it is indicated.

Method I:

Instrument: Waters Acquity UPLCMS SingleQuad; Column: Acquity UPLC BEH C₁₈ 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 mL/min; T: 60° C.; DAD scan: 210-400 nm.

Method II:

Instrument: Waters Acquity UPLCMS SingleQuad; Column: Acquity UPLC BEH C₁₈ 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; T: 60° C.; DAD scan: 210-400 nm.

Method III:

Instrument: Waters Acquity UPLCMS Single Quad; column: BEH C₁₈ 1.7 μm, 50×2.1 mm; eluent A: water+0.05% formic acid (99%); eluent B: acetonitrile+0.05% formic acid (99%); gradient: 0-0.2 2% B, 0.2-1.7 2-90% B, 1.7-1.9 90% B; 1.9-2.0 90-2% B, 2.0-2.5 2% B, flow 1.3 mL/min; T: 60° C.; DAD scan: 200-400 nm.

Method IV:

Instrument: Agilent 1290 UPLCMS 6230 TOF; column: BEH C₁₈ 1.7 μm, 50×2.1 mm; eluent A: water+0.05% formic acid (99%); eluent B: acetonitrile+0.05% formic acid (99%); gradient: 0-1.7 2-90% B, 1.7-2.0 90% B; flow 1.2 ml/min; temperature: 60° C.; DAD scan: 190-400 nm.

Method A:

Instrument: SHIMADZU LCMS—UFLC 20-AD—LCMS 2020 MS detector; Column: Shim-pack XR-ODS, 2.2 μm, 3.0×50 mm; eluent A: water+0.05 vol % trifluoroacetic acid, eluent B: acetonitrile T 0.05 vol % trifluoroacetic acid; gradient: assigned for each compound; flow 1.5 mL/min; T: 40° C.; PDA scan: 190-400 nm.

Method B:

Instrument: SHIMADZU LCMS—UFLC 20-AD—LCMS 2020 MS detector; column: Ascentis Express C₁₈ 2.7 μm, 50×3.0 mm; eluent A: water+0.05 vol % trifluoroacetic acid, eluent B: acetonitrile+0.05 vol % trifluoroacetic acid; gradient: assigned for each compound; flow 1.2 mL/min; T: 40° C.; PDA scan: 190-400 nm.

Method C:

Instrument: SHIMADZU LCMS—UFLC 20-AD—LCMS 2020 MS detector; Column: CORTECS C₁₈ 2.7 μm, 50×2.1 mm; eluent A: water+0.09 vol % formic acid, eluent B: acetonitrile+0.10 vol % formic acid; gradient: assigned for each compound; flow 1.2 mL/min; T: 40° C.; PDA scan: 190-400 nm.

Method D:

Instrument: SHIMADZU LCMS—UFLC 20-AD—LCMS 2020 MS detector; Column: Kinetex EVO C₁₈ 2.6 μm, 50×3.0 mm; eluent A: water+0.05 vol % ammonium hydrogencarbonate, eluent B: acetonitrile; gradient: assigned for each compound; flow 1.5 mL/min; T: 40° C.; PDA scan: 190-400 nm.

Method E:

Instrument: SHIMADZU LCMS—UFLC 20-AD—LCMS 2020 MS detector; Column: CORTECS C₁₈ 2.7 μm, 50×2.1 mm; eluent A: water+0.1 vol % formic acid, eluent B: acetonitrile+0.10 vol % formic acid; gradient: assigned for each compound; flow 1.2 mL/min; T: 40° C.; PDA scan: 190-400 nm.

Method F:

Instrument: SHIMADZU LCMS—UFLC 20-AD—LCMS 2020 MS detector; Column: Kinetex EVO C₁₈ 2.6 μm, 50×3.0 mm; eluent A: water+0.03 vol % ammonium hydroxide, eluent B: acetonitrile; gradient: assigned for each compound; flow 1.5 mL/min; T: 40° C.; PDA scan: 190-400 nm.

Method G:

Instrument: Waters Autopurification MS SingleQuad; column: Waters XBrigde C₁₈ 5 μm 100×3 0 mm; water+0.1 vol-% formic acid (99%), eluent B: acetonitrile; Gradient: 0-5.5 min 5-100% B; flow 70 mL/min; Temperature: 25° C.; DAD scan: 210-400 nm.

### Experimental Section—General Procedures

General Procedures:

In the following general procedures, solvent or reagent quantifications given in mL/mmol refer to the starting material of which 1.0 equivalents were employed.

GP1: Formation of the Carbamate

To an ice-cooled solution of the aniline (1.0 eq.) in pyridine (1.5 mL/mmol), the commercially available either with ethyl chloroformiate (CAS: 541-41-3, 1.2 eq.) or methyl carbonochloridoate (CAS: 79-22-1) was added dropwise. After complete addition, the corresponding suspension was stirred at 0° C. for 2 h, or until the reaction was complete. Upon reaction completion, the reaction mixture was diluted with an aq. solution of 1 M HCl (10 mL/mmol). The resulting precipitate (carbamate) was collected by filtration and washed with water and dried under vacuum overnight and used without any further purification step in the next reaction step.

GP2: Formation of the Pyrimidine Diones

To a cooled suspension of commercially available NaH (CAS: 7440-23-5, 60% in mineral oil, 1.5 eq.) in DMF (2.6 mL/mmol), the acrylate ester (1.1 eq) was added dropwise. The reaction mixture was stirred at RT for around 40 min (until no formation of gas was observed anymore). Then the carbamate (1.0 eq., dissolved in a sufficient amount of DMF) was added and the reaction solution was heated to 90° C. for 18 h-20 h, or until the reaction was complete. Upon reaction completion, the mixture was poured into water, and the resulting precipate was filtered off, and the aq. solution was acidified until pH=3 with an aq. solution 2 M HCl (3.5 mL/mmol). The solution was then diluted with DCM. The organic phase was extracted twice. The combined organic layers were washed with brine and dried with sodium sulfate. After filtration, the solvent was removed under vacuum. The resulted residue was purified using either preparative HPLC (basic) giving the desired cyclized product.

GP3: Ullmann Coupling

The aryl bromide (1.0 eq.), the commercially available phenol (1.1-1.20 eq.), the commercially available ligand N,N-dimethylglycine (CAS: 1118-68-9, 0.3 eq.), commercially available copper (I) iodide (0.135 eq.) and commercially available cesium carbonate (2.4-2.5 eq.) were heated to 140° C. in DMF (3.9 mL/mmol) under argon atmosphere for 18-20 h, or until the reaction was complete. Upon reaction completion, the mixture was cooled and the mixture was diluted with aq. ammonia solution and further stirred at RT for 10 min. Subsequently, ethyl acetate and water were added, and the layers were very carefully separated. The aqueous layer was extracted with ethyl acetate. The combined organic layers were washed with brine and dried with sodium sulfate. After filtration, the solvent was removed under vacuum. The resulted residue was purified using H PLC.

GP4: SN Aromatic

The fluorinated arene (1.0 eq.), commercially available potassium carbonate (2.5 eq.), the phenol (1.0-1.2. eq.) were suspended in DMSO (6.2 mL/mmol), and the reaction was heated to 110° C. for 20 h, or until the reaction was complete. Upon reaction completion, the reaction was filtered and purified by HPLC giving the desired product.

### Experimental Section—Intermediates

### INT-1

### Ethyl 4-chloro-4,4-difluoro-3-oxobutanoate

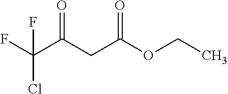

To a solution of ethyl acetate (2.2 g, 25.2 mmol) in tetrahydrofuran (50 mL) was added lithium diisopropylamide (9.5 mL, 2 mol/L in THF) at −78° C. and the resulting mixture was stirred at this temperature for 30 min. Then ethyl 2-chloro-2,2-difluoroacetate (2.0 g, 12.6 mmol) was added to the above mixture at −78° C. and the resulting solution was stirred at RT for overnight under nitrogen atmosphere. Upon completion of the reaction, aq. ammonium chloride solution was added at 0° C. and the resulting mixture was extracted with ethyl acetate. The combined organic layer was dried over anhydrous sodium sulfate and the solvent was removed in vacuo to give the title compound (3.0 g, 59% yield, 50% purity) as light red oil. LC-MS (Method D, 0-1.80 min 5-95% B): Rₜ=0.69 min; MS (ESIneg): m/z=199 (M−H)⁻.

### INT-2

### Ethyl 3-amino-4-chloro-4,4-difluorobut-2-enoate

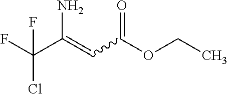

To a solution of aforementioned ethyl 4-chloro-4,4-difluoro-3-oxobutanoate (INT-1, 3.0 g, 7.5 mmol, 50% purity) in methanol (50 mL) was added ammonium acetate (2.3 g, 29.9 mmol). The resulting mixture was stirred at RT for overnight. Upon completion of the reaction, the solvent was removed in vacuo and the residue was purified by silica gel column chromatography (petroleum ether:ethyl acetate=10:1) to give the title compound (800 mg, 48%) as light yellow oil. LC-MS (Method B, 0-1.80 min 5-95% B): Rₜ=1.01 min; MS (ESIpos): m/z=200 (M+H)⁺.

### INT-3

### Ethyl 3-amino-4,4-difluoro-4-phenylbut-2-enoate

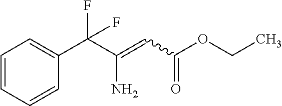 [SMILES: CCOC(=O)C=C(N)C(F)(F)c1ccccc1]

To a solution of diisopropylamine (4.0 mL, 29.41 mmol) in THF (95 mL) was added n-butyllithium (2.5 M in hexane) (11.7 mL, 29.41 mmol) at −40° C., and the mixture was allowed to stir at −10° C. for 30 minutes. Again, the mixture was cooled to −50° C., then ethyl acetate (2.3 mL, 39.2 mmol) was added at −50° C. and the resulting mixture was stirred at this temperature for 30 min. Then a solution of commercially available 2,2-difluoro-2-phenylacetonitrile (CAS: 2002-72-4, 3.0 g, 19.6 mmol) in THF (5 mL) was added to the above mixture at −78° C. and the resulting solution was stirred for the same temperature for 2 h. After completion of starting material, the reaction was stopped by adding aq. saturated aqueous ammonium chloride solution at 0° C., and the mixture was extracted with ethyl acetate. The combined organic layer was dried over anhydrous sodium sulfate, filtered and the solvent was removed in vacuo. The residue was purified with silica gel column chromatography (petroleum ether:ethyl acetate=8:1) to give the title compound (3.16 g, 65%) as a yellow oil. ¹H-NMR (400 MHz, DMSO-d₆): δ [ppm]=1.18 (t, 3H), 4.05 (q, 2H), 4.70 (s, 1H), 7.50-7.57 (m, 5H), 7.61-7.63 (m, 2H); LC-MS (Method B, 0-2.00 min 10-95% B): Rt=1.16 min; MS (ESIpos): m/z=242 (M+H)⁺.

### INT-4

### 4-Amino-5-fluoro-2-(2-methylphenoxy)benzonitrile

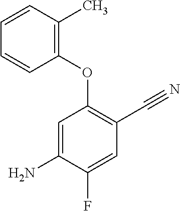 [SMILES: Cc1ccccc1Oc1cc(N)c(F)cc1C#N]

To a slurry of sodium hydride (60% in mineral oil, 1.50 g, 36.9 mmol) in 1-methyl-2-pyrrolidinone (100 mL) were added o-cresol (5.30 g, 49.3 mmol) and commercially available 4-amino-2,5-difluorobenzonitrile (CAS: 112279-61-5, 4.0 g, 24.6 mmol) at 0° C. The resulting mixture was stirred at 100° C. for overnight under nitrogen atmosphere. After cooling to RT, aq. ammonium chloride solution was added at 0° C. and the organic solvent was removed in vacuo. The residue was diluted with water and the resulting mixture was extracted with ethyl acetate. The combined organic layer was dried over anhydrous sodium sulfate and concentrated in vacuo. The residue was purified by silica gel column chromatography (petroleum ether:ethyl acetate=4:1) to give the title compound (3 g, 45%) of the product as a yellow solid. LC-MS (Method A, 0-2.00 min 10-95% B): Rₜ=1.22 min; MS (ESIpos): m/z=243 (M+H)+

### INT-5

### 4-Amino-2-(2-methylphenoxy)benzonitrile

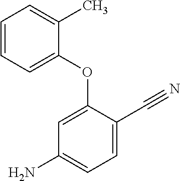 [SMILES: Cc1ccccc1Oc1cc(N)ccc1C#N]

To a solution of commercially available 4-amino-2-bromobenzonitrile (CAS: 53312-82-6, 15 g, 73.8 mmol), in 1-methyl-2-pyrrolidinone (120 mL) were added o-cresol (15.9 g, 147.7 mmol), cesium carbonate (72.2 g, 221.5 mmol), copper (i) iodide (3.7 g, 36.9 mmol), and commercially available 2,2,6,6-tetramethylheptane-3,5-dione (CAS: 1118-71-4, 13.6 g, 73.8 mmol). The resulting mixture was stirred at 120° C. overnight under nitrogen atmosphere. After cooled to RT, the solvent was removed in vacuo and the residue was diluted with water. The resulting mixture was extracted with ethyl acetate and the combined organic layer was dried over anhydrous sodium sulfate. The solvent was removed in vacuo and the residue was purified by silica gel column chromatography (petroleum ether:ethyl acetate=3:1) to give the title compound (14.0 g, 76%) as a yellow solid. LC-MS (Method B, 0-3.00 min 5-95% B): Rₜ=1.39; MS (ESIpos): m/z=225 (M+H)⁺.

### INT-6

### 4-Amino-5-iodo-2-(2-methylphenoxy)benzonitrile

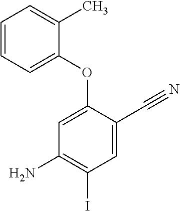 [SMILES: Cc1ccccc1Oc1cc(N)c(I)cc1C#N]

To a solution of aforementioned 4-amino-2-(2-methylphenoxy)benzonitrile (INT-5, 1.0 g, 4.0 mmol), in acetic acid (50 mL) was added N-iodosuccinimide (903 mg, 4.0 mmol), and the resulting mixture was stirred at RT overnight. Upon completion of the reaction, water was added and the resulting mixture was extracted with ethyl acetate. The combined organic layer was dried over anhydrous sodium sulfate and concentrated in vacuo. The residue was purified by silica gel column chromatography (petroleum ether:ethyl acetate=5:1) to give the title compound (900 mg, 64%) as a yellow solid. LC-MS (Method D, 0-3.00 min 10-95% B): Rₜ=1.74; MS (ESIpos): m/z=351 (M+H)⁺.

### INT-7

### 4-Amino-5-bromo-2-(2-methylphenoxy)benzonitrile

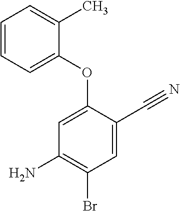 [SMILES: Cc1ccccc1Oc1cc(N)c(Br)cc1C#N]

To a solution of aforementioned 4-amino-2-(2-methylphenoxy)benzonitrile (INT-5, 5.0 g, 19.6 mmol, 88% purity) in acetic acid (100 mL) was added N-bromosuccinimide (3.5 g, 19.6 mmol) and the resulting mixture was stirred at RT overnight. Upon completion of the reaction, the solvent was removed in vacuo and the residue was re-dissolved with water. The resulting mixture was extracted with ethyl acetate and the combined organic layer was dried over anhydrous sodium sulfate. The solvent was removed in vacuo and the residue was purified by silica gel column chromatography (petroleum ether:ethyl acetate=3:1) to give the desired product (5.50 g, 80%) as light yellow oil. LC-MS (Method E, 0-1.50 min 5-95% B): Rₜ=0.93; MS (ESIpos): m/z=303 (M+H)⁺.

### INT-8

### 4-Amino-2-(2-chlorophenoxy)-5-fluorobenzonitrile

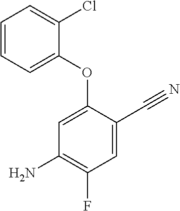 [SMILES: N#Cc1cc(F)c(N)cc1Oc1ccccc1Cl]

This intermediate can be synthesized in analogy to the aforementioned INT-4 from commercially available 4-amino-2,5-difluorobenzonitrile and commercially available 2-chlorophenol.

### INT-9

### 4-Amino-5-chloro-2-(2-methylphenoxy)benzonitrile

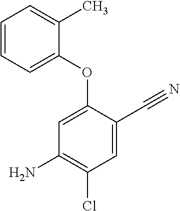 [SMILES: Cc1ccccc1Oc1cc(N)c(Cl)cc1C#N]

To a solution of aforementioned 4-amino-2-(2-methylphenoxy)benzonitrile (INT-5, 3.0 g, 12.0 mmol) in acetic acid (100 mL) was added N-chlorosuccinimide (1.6 g, 12.0 mmol), and the resulting mixture was stirred at RT overnight. Upon completion of the reaction, the solvent was removed in vacuo and the residue was re-dissolved with water. The resulting mixture was extracted with ethyl acetate and the combined organic layer was dried over anhydrous sodium sulfate. The solvent was removed in vacuo and the residue was purified by silica gel column chromatography (petroleum ether:ethyl acetate=8:1) to give 2.1 g (57% yield) of the title compound as a yellow solid. LC-MS (Method A, 0-1.80 min 5-95% B): Rₜ=1.21 min; MS (ESIpos): m/z=259 (M+H)⁺.

### INT-10

### 4-Amino-2-(2,6-dimethylphenoxy)benzonitrile

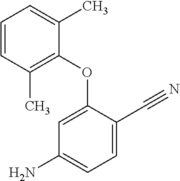 [SMILES: Cc1cccc(C)c1Oc1cc(N)ccc1C#N]

To a solution of commercially available 4-amino-2-bromobenzonitrile (CAS: 53312-82-6, 2.0 g, 10.2 mmol) in 1-methyl-2-pyrrolidinone (60 mL) were added 2,6-dimethylphenol (3.7 g, 30.4 mmol), copper (i) chloride (0.5 g, 5.1 mmol), cesium carbonate (6.60 g, 20.3 mmol) and commercially available 2,2,6,6-tetramethylheptane-3,5-dione (CAS: 1118-71-4, 1.90 g, 10.2 mmol). The resulting mixture was stirred at 110° C. overnight under nitrogen atmosphere. After cooling to RT, the solvent was removed in vacuo and the residue was diluted with water. The resulting mixture was extracted with ethyl acetate and the combined organic layer was dried over anhydrous sodium sulfate. The solvent was removed in vacuo and the residue was purified by silica gel column chromatography (petroleum ether:ethyl acetate=4:1) to give the title compound (750 mg, 27%) as light yellow oil.

### INT-11

### 4-Amino-5-chloro-2-(2,6-dimethylphenoxy)benzonitrile

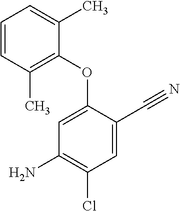 [SMILES: Cc1cccc(C)c1Oc1cc(N)c(Cl)cc1C#N]

To a solution of aforementioned 4-amino-2-(2,6-dimethylphenoxy)benzonitrile (INT-10, 750 mg, 2.8 mmol) in acetic acid (20 mL) was added N-chlorosuccinimide (378 mg, 2.8 mmol). The resulting mixture was stirred at RT for overnight. Upon completion of the reaction, the solvent was removed in vacuo and the residue was diluted with water. The resulting mixture was extracted with ethyl acetate and the combined organic layer was dried over anhydrous sodium sulfate. The solvent was removed in vacuo and the residue was purified by silica gel column chromatography (petroleum ether:ethyl acetate=4:1) to give the title compound (600 mg, 62% yield, 80% purity) as a light yellow solid. LC-MS (Method A, 0-5.00 min 5-100% B): Rₜ=2.06 min; MS (ESIpos): m/z=273 (M+H)⁺.

### INT-12

### 4-Amino-5-hydroxy-2-(o-tolyloxy)benzonitrile

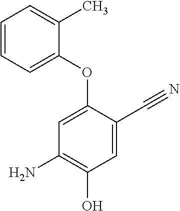 [SMILES: Cc1ccccc1Oc1cc(N)c(O)cc1C#N]

To a solution of 4-amino-5-bromo-2-(o-tolyloxy)benzonitrile (INT-7, 1.70 g, 4.80 mmol) in 1,4-dioxane (50 mL) were added bis(pinacolato)diboron (2.4 g, 9.50 mmol), potassium acetate (1.40 g, 14.3 mmol) and Pd(dppf)Cl₂ (349 mg, 0.5 mmol). The resulting mixture was stirred at 90° C. overnight under nitrogen atmosphere. After cooling to RT, hydrogen peroxide (486 mg, 14.3 mmol, 30% aq. solution) was added and the resulting mixture was stirred at RT for another 1 h. Upon completion of the reaction, the solvent was removed in vacuo and the residue was diluted with water. The resulting mixture was extracted with ethyl acetate and the combined organic layer was dried over anhydrous sodium sulfate. The solvent was removed in vacuo and the residue was purified by silica gel column chromatography (petroleum ether:ethyl acetate=2:1) to give the title compound (800 mg, 63%) as brown oil. LC-MS (Method A, 0-1.80 min 5-95% B): Rₜ=1.04 min; MS (ESIpos): m/z=241 (M+H)⁺.

### INT-13

### 4-Amino-5-propoxy-2-(o-tolyloxy)benzonitrile

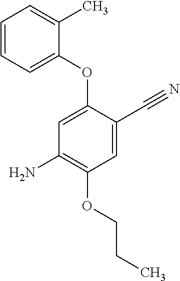 [SMILES: CCCOc1cc(C#N)c(Oc2ccccc2C)cc1N]

To a solution of aforementioned 4-amino-5-hydroxy-2-(o-tolyloxy)benzonitrile (INT-12, 400 mg, 1.5 mmol, in 10 mL DMF) were added cesium carbonate (976 mg, 3.0 mmol) and 1-iodopropane (382 mg, 2.2 mmol). The resulting mixture was stirred at RT for 2 h. Upon completion of the reaction, the solvent was removed in vacuo and the residue was diluted with water. The resulting mixture was extracted with ethyl acetate and the combined organic layer was dried over anhydrous sodium sulfate. The solvent was removed in vacuo and the residue was purified by silica gel column chromatography (petroleum ether:ethyl acetate=2:1) to give the title compound (310 mg, 78%) as a light yellow solid. LC-MS (Method A, 0-3.00 min 5-95% B): Rₜ=1.85 min; MS (ESIpos): m/z=283 (M+H)⁺.

### INT-14

### 2-Bromo-4-nitronaphthalen-1-amine

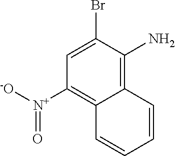

To a stirred solution of commercially available 4-nitronaphthalen-1-amine (CAS: 776-34-1, 250 g, 1.33 mol) in acetonitrile (4 L) were added ammonium acetate (10.2 g, 0.132 mol) and N-bromosuccinimide (260 g, 1.46 mol, portionwise) at 10° C. The resulting mixture was allowed to stir at RT for 1.5 h. A solid precipitated during the reaction, which was filtered, washed with water, and dried over vacuum giving the title compound (300 g, 86%) as a pale yellow solid. LC-MS (Method C, 0-2.00 min 10-95% B): Rₜ=2.00 min; MS (ESIpos): m/z=267 (M+H)⁺.

### INT-15

### 2-(2-Methylphenoxy)-4-nitronaphthalen-1-amine

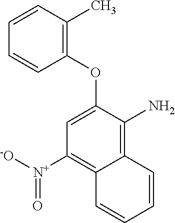

To a solution of aforementioned 2-bromo-4-nitronaphthalen-1-amine (INT-14, 21.0 g, 74.7 mmol) in 1-methyl-2-pyrrolidinone (150 mL) were added o-cresol (80.8 g, 746 mmol), copper (i) chloride (7.40 g, 74.7 mmol), cesium carbonate (48.6 g, 149.3 mmol) and 2,2,6,6-tetramethylheptane-3,5-dione (27.4 g, 149 mmol). The resulting mixture was stirred at 120° C. overnight under nitrogen atmosphere. After cooling to RT, the solvent was removed in vacuo and the residue was diluted with water. The resulting mixture was extracted with ethyl acetate and the combined organic layer was dried over anhydrous sodium sulfate. The solvent was removed in vacuo and the residue was purified by silica gel column chromatography (petroleum ether:ethyl acetate=2:1) to give 5.5 g (90% purity, 23% yield) of the title compound as a brown solid. LC-MS (Method B, 0-2.00 min 10-95% B): Rₜ=1.04 min; MS (ESIpos): m/z=295 (M+H)⁺.

### INT-16

### 1-Bromo-2-(2-methylphenoxy)-4-nitronaphthalene

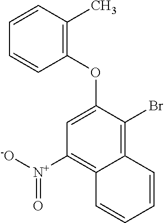

To a solution of aforementioned 2-(2-methylphenoxy)-4-nitronaphthalen-1-amine (INT-15, 5.5 g, 16.8 mmol) in acetonitrile (80 mL) were added copper(II) bromide (4.9 g, 21.9 mmol) and tert-butyl nitrite (2.3 g, 21.9 mmol). The resulting mixture was stirred at 70° C. for 2 h under nitrogen atmosphere. After cooled to RT, the solvent was removed in vacuo and the residue was purified by silica gel column chromatography (petroleum ether:ethyl acetate=10:1) to give the title compound (3.0 g, 45%) as a brown solid. ¹H-NMR (400 MHz, DMSO-d₆): δ [ppm]=2.28 (s, 3H), 6.92 (d, 1H), 7.18 (t, 1H), 7.25 (t, 1H), 7.41 (d, 1H), 7.81-7.85 (m, 2H), 7.91 (t, 1H), 8.35 (d, 1H), 8.41 (d, 1H).

### INT-17

### 2-(2-Methylphenoxy)-4-nitronaphthalene-1-carbonitrile

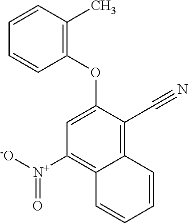

To a solution of aforementioned 1-bromo-2-(2-methylphenoxy)-4-nitronaphthalene (INT-16, 3.0 g, 7.5 mmol) in DMF (20 mL) were added zinc cyanide (2.7 g, 22.6 mmol), 1,2-bis(dimethylamino)ethane (876 mg, 7.5 mmol), Pd₂(dba)₃ (690 mg, 0.8 mmol) and Xantphos (873 mg, 1.5 mmol). The resulting mixture was irradiated with microwaves for 10 min at 180° C. After cooling to RT, water was added and the resulting mixture was extracted with ethyl acetate. The combined organic layer was dried over anhydrous sodium sulfate and concentrated in vacuo. The residue was purified by silica gel column chromatography (petroleum ether:ethyl acetate=10:1) to give the title compound (720 mg, 25%) as a brown solid.

### INT-18

### 4-Amino-2-(2-methylphenoxy)naphthalene-1-carbonitrile

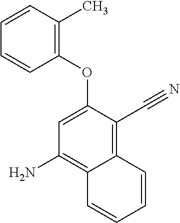 [SMILES: Cc1ccccc1Oc1cc(N)c2ccccc2c1C#N]

To a solution of aforementioned 2-(2-methylphenoxy)-4-nitronaphthalene-1-carbonitrile (INT-17, 720 mg, 80% purity, 1.9 mmol) in acetic acid (40 mL) was added iron powder (1.6 g, 28.4 mmol) and the resulting mixture was stirred at RT for 2 h. Upon completion of the reaction, water was added and the resulting mixture was extracted with ethyl acetate. The combined organic layer was washed with water, brine, dried over anhydrous sodium sulfate and concentrated in vacuo. The residue was purified by silica gel column chromatography (petroleum ether:ethyl acetate=2:1) to give the title compound (550 mg, 80% purity, 84%) as a yellow solid. LC-MS (Method B, 0-2.00 min 10-95% B): Rₜ=1.29 min; MS (ESIpos): m/z=275 (M+H)⁺.

### INT-19

### 1-Chloro-2-(2-methylphenoxy)-4-nitronaphthalene

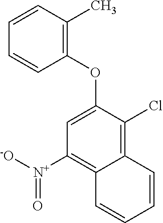

To a solution of aforementioned 2-(2-methylphenoxy)-4-nitronaphthalen-1-amine (INT-15, 55 g, 0.187 mol) in acetonitrile (900 mL) were added copper(II) chloride (37.7 g, 0.280 mol) and tert-butyl nitrite (28.9 g, 0.280 mol). The resulting mixture was stirred at 50° C. for 2 h under nitrogen atmosphere. After completion of starting material, reaction mixture was concentrated under vacuum and the residue was purified with silica gel column chromatography (petroleum ether:ethyl acetate=10:1) to give the title compound (20 g, 34%) as an orange solid. LC-MS (Method E, 0-3.00 min 5-95% B): Rt=1.59 min; MS (ESIpos): m/z=284 (M+H)⁺.

### INT-20

### 4-Chloro-3-(2-methylphenoxy)naphthalen-1-amine

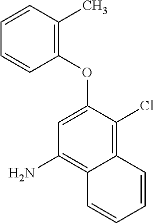 [SMILES: Cc1ccccc1Oc1cc(N)c2ccccc2c1Cl]

To a solution of aforementioned 1-chloro-2-(2-methylphenoxy)-4-nitronaphthalene (INT-19, 20 g, 63.9 mmol) in acetic acid (600 mL) was added iron powder (21.5 g, 384 mmol). The resulting mixture was stirred at RT for 16 h. After completion of starting material, reaction mixture was filtered, concentrated the filtrate to obtain the residue which was partitioned between ethyl acetate (250 mL) and water (250 mL). The organic phase was washed with brine solution, dried over anhydrous sodium sulfate, filtered and concentrated in vacuum to afford the title compound (15 g, crude) as a brown gummy solid. MS(ESIpos): m/z=284 (M+H)⁺.

### INT-21

### 4-Amino-2-(2,6-dimethylphenoxy)-5-fluorobenzonitrile

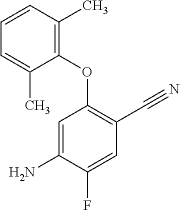 [SMILES: Cc1cccc(C)c1Oc1cc(N)c(F)cc1C#N]

To a suspension of sodium hydride (520 mg, 13.0 mmol, 60% in mineral oil) in 20 mL of 1-methyl-2-pyrrolidinone were added commercially available 2,6-dimethylphenol (CAS: 676-26-1, 1.6 g, 13.0 mmol) and commercially available 4-amino-2,5-difluorobenzonitrile (CAS: 112279-61-5, 1.0 g, 6.5 mmol) at 0° C. The resulting mixture was heated to 100° C. overnight under nitrogen atmosphere. Upon completion of the reaction, aq. ammonium chloride solution was added at 0° C. and all volatiles were removed in vacuo. The residue was re-dissolved with water and the resulting mixture was extracted with ethyl acetate. The combined organic layer was dried over anhydrous sodium sulfate and concentrated in vacuo. The residue was purified by silica gel column chromatography (petroleum ether:ethyl acetate=4:1) to give the title compound (600 mg, 36%, 80% purity) as light yellow oil. LC-MS (Method I): Rₜ=0.88 min; MS (ESIpos): m/z=257 (M+H)⁺.

TABLE 2

| Intermediate No & | |
|---|---|
| starting material | |
| (s.m.) | Structure, lUPAC-Name and analytics |
| INT-22 s.m.: INT-4 | Ethyl [4-cyano-2-fluoro-5-(2-methylphenoxy)phenyl]carbamate 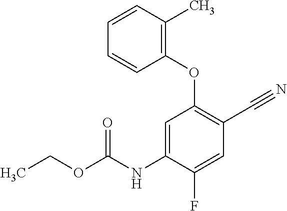 [SMILES: CCOC(=O)Nc1cc(Oc2ccccc2C)c(C#N)cc1F] |
| | ¹H-NMR (400 MHz, DMSO-d₆) δ [ppm]: 1.075 (0.68), 1.093 (1.52), |
| | 1.111 (0.72), 1.145 (5.59), 1.163 (12.22), 1.180 (5.73), 2.160 (16.00), |
| | 2.518 (0.94), 2.522 (0.59), 4.036 (1.76), 4.054 (5.61), 4.072 (5.60), |
| | 4.090 (1.82), 4.110 (0.68), 4.127 (0.66), 7.044 (1.82), 7.046 (1.87), |
| | 7.063 (2.20), 7.066 (2.18), 7.195 (0.78), 7.198 (0.82), 7.214 (2.07), |
| | 7.216 (2.04), 7.232 (1.55), 7.234 (1.39), 7.272 (1.09), 7.276 (1.28), |
| | 7.291 (1.49), 7.296 (1.60), 7.311 (0.63), 7.314 (0.60), 7.374 (1.79), |
| | 7.376 (1.76), 7.393 (1.52), 7.406 (2.58), 7.422 (2.53), 7.876 (3.52), |
| | 7.902 (3.53), 9.943 (2.47). LC-MS (Method I): Rₜ = 1.29 min; MS |
| | (ESIpos): m/z = 314 [M + H]⁺. |
| INT-23 s.m. INT-8 | Ethyl [5-(2-chlorophenoxy)-4-cyano-2-fluorophenyl]carbamate 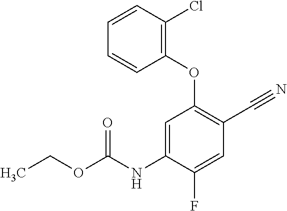 [SMILES: CCOC(=O)Nc1cc(Oc2ccccc2Cl)c(C#N)cc1F] |
| | ¹H-NMR (400 MHz, DMSO-d₆) δ [ppm]: 1.077 (1.96), 1.094 (4.46), |
| | 1.112 (2.14), 1.151 (7.14), 1.169 (16.00), 1.187 (7.54), 1.198 (0.47), |
| | 1.260 (0.50), 2.518 (7.96), 2.523 (6.41), 4.046 (2.26), 4.064 (7.50), |
| | 4.082 (7.52), 4.089 (0.99), 4.099 (2.38), 4.107 (2.21), 4.125 (2.11), |
| | 4.143 (0.67), 7.285 (0.65), 7.292 (0.46), 7.296 (0.52), 7.301 (0.65), |
| | 7.329 (2.06), 7.332 (2.77), 7.336 (1.73), 7.340 (1.30), 7.343 (0.73), |
| | 7.348 (2.30), 7.351 (3.97), 7.355 (4.56), 7.360 (2.34), 7.367 (0.56), |
| | 7.375 (2.43), 7.379 (1.75), 7.445 (2.16), 7.450 (2.90), 7.455 (3.76), |
| | 7.465 (3.46), 7.470 (4.13), 7.485 (1.38), 7.488 (1.34), 7.645 (0.44), |
| | 7.648 (0.47), 7.664 (3.37), 7.668 (2.84), 7.685 (2.78), 7.688 (2.15), |
| | 7.918 (4.52), 7.945 (4.50), 8.143 (0.59), 8.166 (0.59), 10.013 (1.73); |
| | LC-MS (Method I): Rₜ = 1.21 min; MS (ESIpos): m/z = 335 [M + H]⁺. |
| INT-24 s.m.: commercially available 4-amino-2- fluoro-5- methoxybenzonitrile (CAS: 1441723-24-5) | Ethyl (4-cyano-5-fluoro-2-methoxyphenyl)carbamate 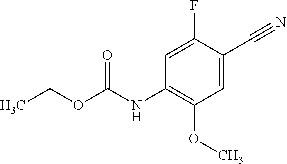 [SMILES: CCOC(=O)Nc1cc(F)c(C#N)cc1OC] |
| | ¹H-NMR (400 MHz, DMSO-d₆) δ [ppm]: 1.229 (7.34), 1.247 (16.00), |
| | 1.265 (7.53), 3.344 (0.98), 4.144 (2.34), 4.162 (7.50), 4.180 (7.40), |
| | 4.197 (2.24), 7.489 (3.87), 7.504 (3.85), 7.924 (4.10), 7.953 (4.08), |
| | 9.143 (2.47); LC-MS (Method I): Rₜ = 1.05 min; MS (ESIpos): m/z = |
| | 239 [M + H]⁺. |
| INT-25 s.m.: commercially available 4-amino-2- fluoro-5- methylbenzonitrile (CAS: 1357942-79-0) | Ethyl (4-cyano-5-fluoro-2-methylphenyl)carbamate 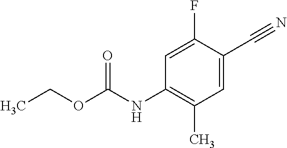 [SMILES: CCOC(=O)Nc1cc(F)c(C#N)cc1C] |
| | ¹H-NMR (400 MHz, DMSO-d₆) δ [ppm]: 1.310 (7.23), 1.328 (16.00), |
| | 1.345 (7.50), 2.283 (13.56), 3.400 (2.26), 4.213 (2.25), 4.230 (7.12), |
| | 4.248 (6.98), 4.266 (2.13), 7.743 (2.31), 7.762 (2.31), 7.831 (3.12), |
| | 7.862 (3.10), 9.402 (2.60); LC-MS (Method I): Rₜ = 1.07 min; MS |
| | (ESIneg): m/z = 221 [M − H]⁻. |
| INT-26 s.m.: INT-5 | Ethyl (4-cyano-3-fluorophenyl)carbamate 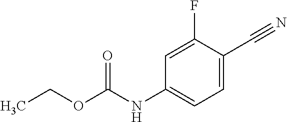 |
| | ¹H-NMR (400 MHz, DMSO-d₆) δ [ppm]: 1.237 (7.36), 1.255 (16.00), |
| | 1.272 (7.46), 2.518 (0.74), 2.523 (0.46), 4.144 (2.23), 4.162 (7.22), |
| | 4.180 (7.19), 4.198 (2.20), 7.344 (2.07), 7.349 (2.04), 7.365 (2.23), |
| | 7.370 (2.23), 7.599 (2.03), 7.603 (1.93), 7.630 (2.00), 7.635 (1.88), |
| | 7.789 (2.24), 7.809 (2.79), 7.811 (2.46), 7.830 (2.09), 10.432 (2.41); |
| | LC-MS (Method II): Rₜ = 1.03 min; MS (ESIneg): m/z = 207 [M − H]⁻. |
| INT-27 s.m.: INT-52 | Ethyl (5-bromo-4-cyano-2-methoxyphenyl)carbamate 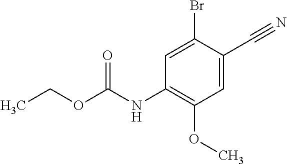 [SMILES: CCOC(=O)Nc1cc(Br)c(C#N)cc1OC] |
| | ¹H-NMR (400 MHz, DMSO-d₆) δ [ppm]: 1.225 (7.33), 1.243 (16.00), |
| | 1.261 (7.56), 3.332 (12.79), 4.133 (2.19), 4.151 (6.89), 4.169 (6.82), |
| | 4.187 (2.13), 7.575 (8.90), 8.252 (8.72), 9.103 (3.64); LC-MS |
| | (Method I): Rₜ = 1.24 min; MS (ESIneg): m/z = 298 [M − H]⁻. |
| INT-28 s.m.: commercially available 5-bromo-4- chloro-2- methoxyaniline (CAS: 102169-94-8) | Ethyl (5-bromo-4-chloro-2-methoxyphenyl)carbamate 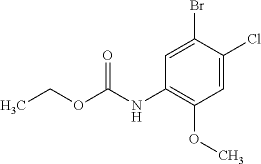 [SMILES: CCOC(=O)Nc1cc(Br)c(Cl)cc1OC] |
| | ¹H-NMR (400 MHz, DMSO-d₆) δ [ppm]: 1.206 (7.39), 1.223 (16.00), |
| | 1.240 (7.63), 4.089 (2.54), 4.107 (8.04), 4.124 (7.94), 4.142 (2.45), |
| | 7.276 (9.31), 8.035 (3.95), 8.742 (3.09); LC-MS (Method I): Rₜ = 1.36 |
| | min; MS (ESIpos): m/z = 308 [M + H]⁺. |

The following intermediates INT-22-28 were prepared by reacting the respective starting materials, as specified below, with commercially available ethyl carbonochloridate (CAS: 541-41-3) according to GP1 (see the preamble of table 3 for a reference protocol).

TABLE 3

| Intermediate No & | |
|---|---|
| starting material | |
| (s.m.) | Structure, lUPAC-Name and analytics |
| INT-29 s.m.: INT-4 | Methyl [4-cyano-2-fluoro-5-(2-methylphenoxy)phenyl]carbamate 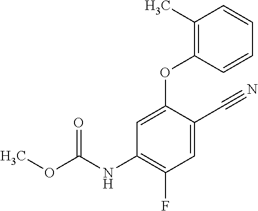 [SMILES: COC(=O)Nc1cc(Oc2ccccc2C)c(C#N)cc1F] |
| | LC-MS (Method B, 0-2.00 min 5-100% B): Rₜ = 1.12 min; MS (ESIpos): |
| | m/z = 301 (M + H)⁺. |
| INT-30 s.m.: INT-18 | Methyl [4-cyano-3-(2-methylphenoxy)naphthalen-1-yl]carbamate 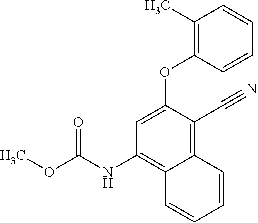 [SMILES: COC(=O)Nc1cc(Oc2ccccc2C)c(C#N)c2ccccc12] |
| | LC-MS (Method D, 0-2.00 min 10-95% B): Rₜ = 1.08 min; MS (ESIpos): |
| | m/z = 333 (M + H)⁺. |
| INT-31 s.m.: INT-7 | Methyl [2-bromo-4-cyano-5-(2-methylphenoxy)phenyl]carbamate 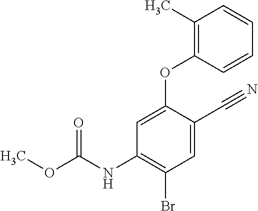 [SMILES: COC(=O)Nc1cc(Oc2ccccc2C)c(C#N)cc1Br] |
| | LC-MS (Method E, 0-1.50 min 5-95% B): Rₜ = 0.96 min; MS (ESIneg): |
| | m/z = 359 |
| INT-32 s.m.: INT-9 | Methyl [2-chloro-4-cyano-5-(2-methylphenoxy)phenyl]carbamate 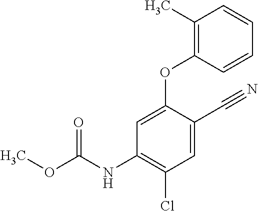 [SMILES: COC(=O)Nc1cc(Oc2ccccc2C)c(C#N)cc1Cl] |
| | LC-MS (Method B, 0-3.00 min 5-95% B): Rₜ = 1.71 min; MS (ESIpos): |
| | m/z = 317 (M + H)⁺. |
| INT-33 s.m.: INT-6 | Methyl 4-cyano-2-iodo-5-(o-tolyloxy)phenylcarbamate 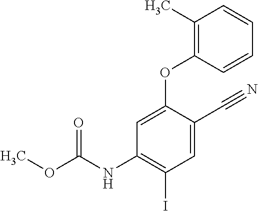 [SMILES: COC(=O)Nc1cc(Oc2ccccc2C)c(C#N)cc1I] |
| | LC-MS (Method F, 0-2.00 min 10-95% B): Rₜ = 1.41 min; MS (ESIneg): |
| | m/z = 407 (M − H)⁻. |
| INT-34 s.m.: INT-20 | Methyl [4-chloro-3-(2-methylphenoxy)naphthalen-1-yl]carbamate 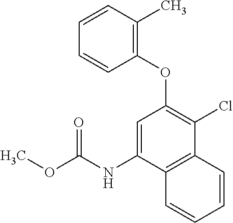 [SMILES: COC(=O)Nc1cc(Oc2ccccc2C)c(Cl)c2ccccc12] |
| | LC-MS (Method E, 0-1.50 min 5-95% B): Rₜ = 1.05 min; MS (ESIpos): |
| | m/z = 342 (M + H)⁺. |
| INT-35 s.m.: INT-11 | Methyl 2-chloro-4-cyano-5-(2,6-dimethylphenoxy)phenylcarbamate 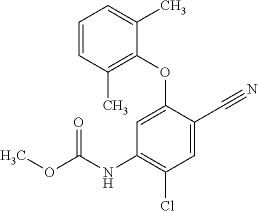 [SMILES: COC(=O)Nc1cc(Oc2c(C)cccc2C)c(C#N)cc1Cl] |
| | LC-MS (Method D, 0-2.00 min 10-95% B): Rₜ = 1.30 min; MS |
| | (ESIneg): m/z = 329 (M − H)⁻. |
| INT-36 s.m.: INT-13 | Methyl 4-cyano-2-propoxy-5-(o-tolyloxy)phenylcarbamate 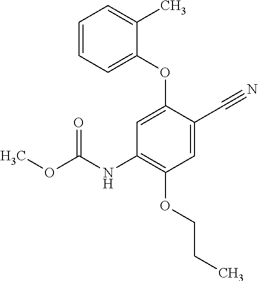 [SMILES: CCCOc1cc(C#N)c(Oc2ccccc2C)cc1NC(=O)OC] |
| | LC-MS (Method E, 0-1.50 min 5-95% B): Rₜ = 1.09 min; MS (ESIpos): |
| | m/z = 341 (M + H)⁺. |
| INT-37 s.m.: INT-21 | Methyl [4-cyano-5-(2,6-dimethylphenoxy)-2-fluorophenyl]carbamate 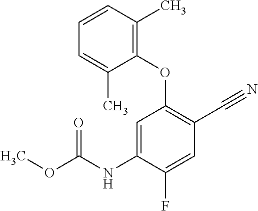 [SMILES: COC(=O)Nc1cc(Oc2c(C)cccc2C)c(C#N)cc1F] |
| | LC-MS (Method A, 0-2.00 min 5-95% B): Rₜ = 1.32 min; MS (ESIpos): |
| | m/z = 315 (M + H)⁺. |

The following intermediates INT-29-37 were prepared in a fashion to similar those in table 1 by reacting the respective starting materials, as specified below, with commercially available methyl carbonochloridoate (CAS: 79-22-1) according to the following example protocol:

According to GP1, to a solution of 4-amino-5-fluoro-2-(2-methylphenoxy)benzonitrile (INT-4, 2.9 g, 10.8 mmol) in pyridine (80 mL) was added methyl carbonochloridate (2.0 g, 21.5 mmol) and the resulting mixture was stirred at room temperature overnight. Upon completion of the reaction, the solvent was removed in vacuo and the residue was purified by Prep-HPLC [eluent A:water, eluent B: acetonitrile; gradient: 22% B to 62% B in 8 min] to give the desired intermediate INT-29 (400 mg, 12%) as a white solid.

TABLE 4

| Intermediate No & | |
|---|---|
| starting materials | |
| (s.m.) | Structure, lUPAC-Name and analytics |
| INT-38 s.m.: INT-27, INT-3 | 2-Bromo-4-{4-[difluoro(phenyl)methyl]-2,6-dioxo-3,6- dihydropyrimidin-1(2H)-yl}-5-methoxybenzonitrile 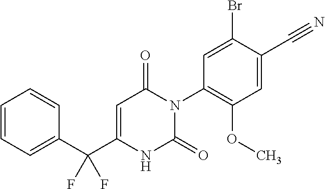 [SMILES: COc1cc(C#N)c(Br)cc1-n1c(=O)cc(C(F)(F)c2ccccc2)[nH]c1=O] |
| | ¹H-NMR (400 MHz, DMSO-d₆) δ [ppm]: 0.852 (0.56), 1.225 (4.96), |
| | 1.243 (10.93), 1.261 (4.85), 1.352 (0.45), 2.074 (1.69), 2.518 (16.00), |
| | 2.523 (11.49), 2.539 (1.80), 2.950 (0.79), 3.073 (1.13), 3.290 (0.68), |
| | 3.804 (7.10), 3.853 (14.42), 3.906 (0.45), 4.135 (1.35), 4.152 (4.51), |
| | 4.170 (4.51), 4.188 (1.24), 6.101 (0.79), 7.585 (5.86), 7.607 (1.92), |
| | 7.734 (1.24), 7.819 (1.58), 7.953 (0.68), 8.257 (5.18), 9.131 (1.69), |
| | 12.229 (1.01); LC-MS (Method I): Rₜ = 1.19 min; MS (ESIneg): m/z = |
| | 447 [M − H]⁻. |
| INT-39 s.m.: INT-24, commercially available ethyl 3- amino-4,4- difluorobut-2-enoate (CAS : 81982-54-9) | 4-[4-(Difluoromethyl)-2,6-dioxo-3,6-dihydropyrimidin-1(2H)-yl]-2- fluoro-5-methoxybenzonitrile 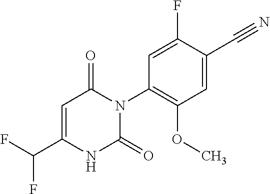 [SMILES: COc1cc(C#N)c(F)cc1-n1c(=O)cc(C(F)F)[nH]c1=O] |
| | ¹H-NMR (400 MHz, DMSO-d₆) δ [ppm]: 1.179 (0.76), 2.518 (1.67), |
| | 2.523 (1.09), 2.540 (1.05), 3.779 (0.75), 3.800 (16.00), 3.892 (0.56), |
| | 6.087 (3.30), 6.517 (0.58), 6.702 (1.02), 6.834 (2.07), 6.967 (0.87), |
| | 7.693 (2.85), 7.716 (2.90), 7.763 (2.44), 7.777 (2.48); LC-MS (Method |
| | I): Rₜ = 0.76 min; MS (ESIneg): m/z = 310 [M − H]⁻. |
| INT-40 s.m.: INT-24, INT-2 | 4-{4-[Chloro(difluoro)methyl]-2,6-dioxo-3,6-dihydropyrimidin- 1(2H)-yl}-2-fluoro-5-methoxybenzonitrile 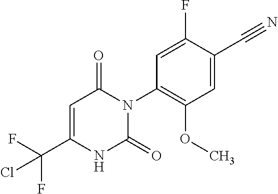 [SMILES: COc1cc(C#N)c(F)cc1-n1c(=O)cc(C(F)(F)Cl)[nH]c1=O] |
| | ¹H-NMR (400 MHz, DMSO-d₆) δ [ppm]: 1.154 (0.40), 1.172 (0.75), |
| | 1.190 (0.40), 1.987 (1.35), 2.518 (3.50), 2.523 (2.29), 2.539 (0.55), |
| | 2.957 (3.17), 2.994 (1.06), 3.337 (2.94), 3.779 (16.00), 3.898 (1.85), |
| | 6.228 (1.62), 6.319 (7.18), 6.475 (2.91), 6.504 (2.87), 7.074 (2.44), |
| | 7.090 (2.30), 7.701 (5.11), 7.724 (5.05), 7.779 (4.76), 7.793 (4.76); LC- |
| | MS (Method III): Rₜ = 0.84 min; MS (ESIpos): m/z = 346 [M + H]⁺. |
| INT-41 s.m.: INT-28, commercially available ethyl 3- amino-4,4- difluorobut-2-enoate (CAS : 81982-54-9) | 3-(5-Bromo-4-chloro-2-methoxyphenyl)-6- (difluoromethyl)pyrimidine-2,4(1H,3H)-dione 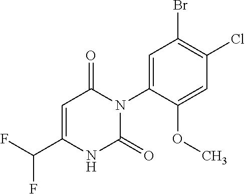 [SMILES: COc1cc(Cl)c(Br)cc1-n1c(=O)cc(C(F)F)[nH]c1=O] |
| | ¹H-NMR (400 MHz, DMSO-d₆) δ [ppm]: 1.158 (2.89), 1.168 (0.44), |
| | 1.176 (5.81), 1.194 (2.86), 1.277 (0.51), 1.295 (1.17), 1.313 (0.54), |
| | 2.326 (1.81), 2.331 (1.30), 2.336 (0.60), 2.518 (7.46), 2.522 (4.70), |
| | 2.539 (1.43), 2.669 (1.81), 2.673 (1.33), 2.678 (0.60), 3.774 (16.00), |
| | 3.872 (4.29), 4.021 (0.86), 4.039 (2.63), 4.056 (2.60), 4.074 (0.83), |
| | 4.308 (0.41), 4.325 (0.41), 4.722 (1.59), 6.018 (2.16), 6.259 (0.67), |
| | 6.381 (0.44), 6.393 (1.52), 6.529 (0.67), 6.661 (0.63), 6.794 (1.30), |
| | 6.927 (0.60), 7.210 (1.56), 7.381 (0.44), 7.471 (5.78), 7.748 (4.44), |
| | 8.169 (0.57), 8.497 (1.81); LC-MS (Method IV): Rₜ = 0.90 min; MS |
| | (ESIpos): m/z = 383 [M + H]⁺. |
| INT-42 s.m.: INT-25, commercially available ethyl 3- amino-4,4,4- trifluorobut-2-enoate (CAS : 372-29-2) | 4-[2,6-Dioxo-4-(trifluoromethyl)-3,6-dihydropyrimidin-1(2H)-yl]-2- fluoro-5-methylbenzonitrile 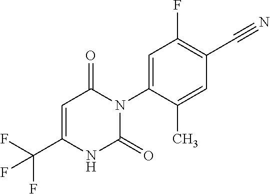 [SMILES: Cc1cc(C#N)c(F)cc1-n1c(=O)cc(C(F)(F)F)[nH]c1=O] |
| | ¹H-NMR (400 MHz, DMSO-d₆) δ [ppm]: 2.002 (1.63), 2.087 (16.00), |
| | 3.348 (1.42), 6.449 (7.38), 6.477 (0.43), 7.649 (3.83), 7.674 (3.77), |
| | 7.989 (3.00), 8.007 (2.99); LC-MS (Method IV): Rₜ = 0.81 min; MS |
| | (ESIpos): m/z = 314 [M + H]⁺. |
| INT-43 s.m.: INT-26, commercially available ethyl 3- amino-4,4,4- trifluorobut-2-enoate (CAS : 372-29-2) | 4-[2,6-Dioxo-4-(trifluoromethyl)-3,6-dihydropyrimidin-1(2H)-yl]-2- fluorobenzonitrile 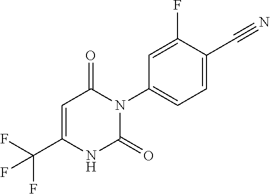 [SMILES: N#Cc1ccc(-n2c(=O)cc(C(F)(F)F)[nH]c2=O)cc1F] |
| | ¹H-NMR (400 MHz, DMSO-d₆) δ [ppm]: 2.518 (5.21), 2.523 (3.29), |
| | 2.990 (1.27), 6.423 (16.00), 7.450 (6.72), 7.455 (6.40), 7.471 (7.04), |
| | 7.475 (6.75), 7.662 (6.82), 7.666 (6.55), 7.688 (6.77), 7.691 (6.38), |
| | 8.066 (6.60), 8.085 (8.30), 8.106 (5.99), 12.709 (0.71); LC-MS (Method |
| | IV): Rₜ = 0.74 min; MS (ESIpos): m/z = 300 [M + H]⁺. |
| INT-44 s.m.: INT-57 commercially available ethyl 3- amino-4,4,4- trifluorobut-2-enoate (CAS : 372-29-2) | 5-[2,6-Dioxo-4-(trifluoromethyl)-3,6-dihydropyrimidin-1(2H)-yl]-3- fluoropyridine-2-carbonitrile 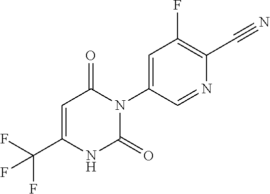 [SMILES: N#Cc1ncc(-n2c(=O)cc(C(F)(F)F)[nH]c2=O)cc1F] |
| | ¹H-NMR (400 MHz, DMSO-d₆) δ [ppm]: 1.365 (0.78), 1.383 (1.79), |
| | 1.400 (0.80), 2.518 (7.12), 2.523 (4.53), 2.539 (1.14), 2.971 (0.44), |
| | 3.088 (5.93), 3.365 (1.42), 4.220 (0.78), 4.238 (0.75), 6.423 (0.65), |
| | 6.468 (0.85), 6.501 (16.00), 6.987 (0.83), 7.196 (1.19), 7.619 (0.52), |
| | 7.624 (0.54), 7.994 (0.65), 7.998 (0.65), 8.026 (0.52), 8.031 (0.52), |
| | 8.287 (7.95), 8.291 (8.10), 8.311 (7.53), 8.315 (7.77), 8.682 (8.72), |
| | 8.685 (9.73), 8.687 (10.12), 8.689 (8.26); LC-MS (Method IV): Rₜ = 0.71 |
| | min; MS (ESIpos): m/z = 301 [M + H]⁺. |
| INT-45 s.m.: INT-28, commercially available ethyl 3- amino-4,4,4- trifluorobut-2-enoate (CAS : 372-29-2) | 3-(5-Bromo-4-chloro-2-methoxyphenyl)-6- (trifluoromethyl)pyrimidine-2,4(1H,3H)-dione 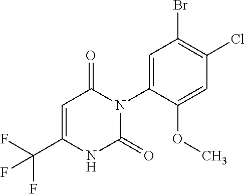 [SMILES: COc1cc(Cl)c(Br)cc1-n1c(=O)cc(C(F)(F)F)[nH]c1=O] |
| | ¹H-NMR (400 MHz, DMSO-d₆) δ [ppm]: 1.155 (0.71), 1.173 (1.55), |
| | 1.190 (0.79), 1.988 (2.63), 2.518 (0.97), 2.523 (0.74), 2.922 (1.02), |
| | 3.784 (16.00), 3.860 (0.44), 4.017 (0.53), 4.035 (0.51), 6.330 (1.02), |
| | 7.485 (5.54), 7.751 (3.23); LC-MS (Method IV): Rₜ = 1.00 min; MS |
| | (ESIpos): m/z = 400 [M + H]⁺. |
| INT-46 s.m.: INT-27, commercially available ethyl 3- amino-5,5,5,4,4- pentafluoropent-2- enoate (CAS: 72850- 56-7) | 2-Bromo-4-[2,6-dioxo-4-(pentafluoroethyl)-3,6-dihydropyrimidin- 1(2H)-yl]-5-methoxybenzonitrile 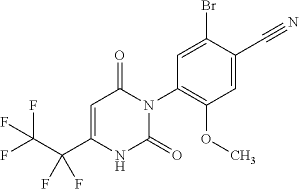 [SMILES: COc1cc(C#N)c(Br)cc1-n1c(=O)cc(C(F)(F)C(F)(F)F)[nH]c1=O] |
| | ¹H-NMR (400 MHz, DMSO-d₆) δ [ppm]: 2.326 (3.20), 2.331 (2.36), |
| | 2.336 (1.07), 2.518 (13.53), 2.522 (8.53), 2.539 (3.93), 2.669 (3.20), |
| | 2.673 (2.36), 2.678 (1.07), 3.818 (16.00), 6.942 (1.57), 7.070 (1.74), |
| | 7.198 (1.57), 7.828 (2.36), 7.906 (0.62); LC-MS (Method IV): Rₜ = 1.00 |
| | min; MS (ESIpos): m/z = 442 [M + H]⁺. |

The following intermediates INT-38-46 were prepared, according to GP2, from the respective carbamates and amino acrylate esters as specified below. Besides from intermediate INT-43 and INT-44 intermediates from this table are assumed to have been obtained as diasteromeric mixtures; their atropisomeric and/or diasteroisomeric ratio was not investigated at that step.

### INT-47

### 1-[4-cyano-2-fluoro-5-(2-methylphenoxy)phenyl]urea

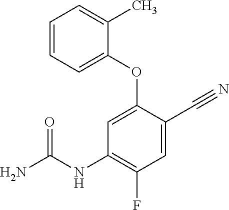 [SMILES: Cc1ccccc1Oc1cc(NC(N)=O)c(F)cc1C#N]

To a solution of aforementioned 4-amino-5-fluoro-2-(2-methylphenoxy)benzonitrile (INT-4, 20 g, 57.8 mmol, 1.0 eq) in acetonitrile (100 mL) was added commercially available chlorosulfonyl-isocyanate (CAS: 1189-71-5, 9.82 g, 69.4 mmol, 6.0 mL, 1.2 eq), the mixture was stirred for 0.5 h at 25° C. Upon reaction completion, the reaction mixture was poured into water (500 mL) and extracted with ethyl acetate (3×200 mL). The combined organic layer was washed with brine (500 mL), dried over sodium sulfate, filtered and concentrated, the residue was triturated by MTBE (50 mL) and filtered, filter cake was dried in vacuo to give the title compound (9.5 g, 56%).

### INT-48

### 5-Fluoro-2-(2-methylphenoxy)-4-(2,4,6-trioxotetrahydropyrimidin-1(2H)-yl)benzonitrile

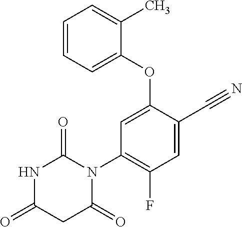 [SMILES: Cc1ccccc1Oc1cc(N2C(=O)CC(=O)NC2=O)c(F)cc1C#N]

A mixture of aforementioned 1-[4-cyano-2-fluoro-5-(2-methylphenoxy)phenyl]urea (INT-47, 6.0 g, 21.0 mmol, 1.0 eq), commercially available dimethyl propanedioate (CAS: 108-59-8, 11.5 g, 87.1 mmol, 10 mL, 4.14 eq) and sodium ethoxide (5.73 g, 84.1 mmol, 4.0 eq) in ethanol (50 mL) was stirred for 6 h at 100° C. Upon reaction completion, the reaction mixture was poured into water (200 mL) and extracted with ethyl acetate (3×200 mL); the combined organic layer was washed with brine (200 mL), dried over sodium sulfate, filtered and concentrated. The residue was triturated by MTBE/PE=1/1 (100 mL) and was then filtered, the filter cake was dried in vacuo to give the title compound (5.50 g, 74%) as a white solid. ¹H-NMR (400 MHz, DMSO-d₆) 5 [ppm]: 11.65 (s, 1H), 8.13 (d, 1H), 7.38 (d, 1H), 7.32-7.29 (m, 1H), 7.25-7.23 (m, 1H), 5.09 (s, 2H), 7.11 (d, 1H), 6.86 (d, 1H), 3.95-3.91 (m, 1H), 3.62-3.53 (m, 1H), 2.18 (s, 3H).

### INT-49

### 1-[4-Cyano-2-fluoro-5-(2-methylphenoxy)phenyl]-2,6-dioxo-1,2,3,6-tetrahydropyrimidine-4-carboxylic acid

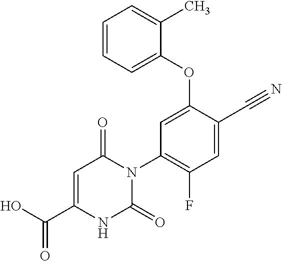 [SMILES: Cc1ccccc1Oc1cc(-n2c(=O)cc(C(=O)O)[nH]c2=O)c(F)cc1C#N]

In a parallel-autoclave reactor were placed 4-(4-Bromo-2,6-dioxo-3,6-dihydropyrimidin-1(2H)-yl)-5-fluoro-2-(2-methylphenoxy)benzonitrile (700 mg, 1.68 mmol, see example 3 below in the Examples part), 1,1′-bis(diphenylphosphino)ferrocene palladium(II)dichloride dichloromethane adduct (137 mg, 168 μmol, 0.1 eq,) suspended in methanol/THF (22 mL, v/v=10:1) with triethylamine (470 μL, 3.4 mmol, 2.0 eq). The mixture was stirred at room temperature under an atmosphere of carbon monoxide (10.5 bar, three times degassed) for 30 min. The pressure was changed to 14.6 bar and then the reaction mixture was heated to 100° C. overnight. Upon reaction completion (new peak detected), the mixture was filtered and purified using HPLC HT to give the title compound (70 mg, 10%) as a white solid. ¹H-NMR (400 MHz, DMSO-d₆) δ [ppm]: 1.168 (0.41), 2.161 (16.00), 2.518 (1.77), 2.523 (1.12), 5.843 (11.75), 7.011 (3.95), 7.026 (4.29), 7.032 (2.33), 7.049 (2.48), 7.052 (2.51), 7.179 (1.15), 7.181 (1.19), 7.197 (2.58), 7.200 (2.48), 7.215 (1.92), 7.219 (1.77), 7.264 (1.29), 7.267 (1.48), 7.282 (1.77), 7.287 (1.90), 7.303 (0.84), 7.306 (0.81), 7.355 (1.92), 7.357 (1.88), 7.374 (1.60), 8.107 (3.84), 8.129 (3.80). LC-MS (Method IV): Rₜ=0.88 min; MS (ESIpos): m/z=382 [M+H]⁺.

### INT-50

### 2-Bromo-5-methoxy-4-nitrobenzamide

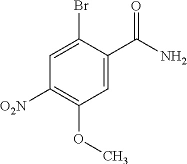

To a solution of commercially available 2-bromo-5-methoxy-4-nitrobenzoic acid (CAS: 1432752-25-4, 50 g, 0.181 mol) in dichloromethane (500 mL) was added oxalyl chloride (46.6 mL, 0.543 mol) at 0° C. The mixture was stirred at room temperature for 3 h and after reaction completion, the solvent was concentrated in vacuo. To the crude residue was added THF (500 ml), and the resulting mixture was purged with ammonia gas at room temperature for 30 min. The reaction was monitored by thin layer chromatography. After completion of the reaction, the reaction mixture was concentrated completely, and to the solid residue was added water and the mixture was stirred for 10 min. The solid was filtered off and was dried under vacuum to give the title compound as light yellow solid (42.1 g, 84%). ¹H-NMR (400 MHz, DMSO-d₆) δ [ppm]: 14.06 (br s, 1H), 8.25 (s, 1H), 7.63 (s, 1H), 3.96 (s, 3H).

### INT-51

### 2-Bromo-5-methoxy-4-nitrobenzonitrile

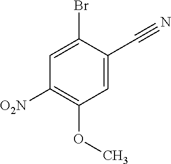

To a stirred solution of aforementioned 2-bromo-5-methoxy-4-nitrobenzamide (INT-50 (42 g, 0.152 mol) in dioxane (420 mL) at 0° C. was added pyridine (121 mL, 1.53 mol) and then, slowly, trifluoroacetic anhydride (107 mL, 0.763 mol). After completion of the addition, the reaction mixture was stirred for 16 h at room temperature. After completion of the reaction (TLC), the reaction was stopped by adding water (500 mL). The obtained solid was filtered, washed with water (500 mL) and dried under vacuum to give the title compound (34 g, 86%) as an off-white solid. ¹H-NMR (400 MHz, DMSO-d₆) δ [ppm]: 8.44 (s, 1H), 8.09 (s, 1H), 3.98 (s, 3H).

### INT-52

### 4-Amino-2-bromo-5-methoxybenzonitrile

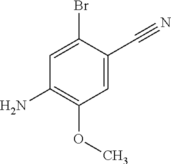

To a stirred solution of aforementioned 2-bromo-5-methoxy-4-nitrobenzonitrile (INT-51, 33 g, 0.128 mol) in acetic acid (165 mL) and ethyl acetate (165 mL) was added iron powder (33 g). The reaction mixture was then heated to reflux for 10 h. After completion of the reaction (TLC), the mixture was cooled to room temperature and was filtered through Celite®. The resulting filtrate was concentrated and saturated aq. sodium bicarbonate solution (300 mL) was added. The mixture was extracted with ethyl acetate (2×500 mL). After concentration of the combined the organic layers, ether (500 mL) was added to the residue and the mixture was stirred for 15 min. The solid was filtered off and dried under vacuum to give the title compound (25.5 g, 87%) as a white solid. ¹H-NMR (400 MHz, DMSO-d₆) δ [ppm]: 7.19 (s, 1H), 6.88 (s, 1H), 6.09 (br s, 2H), 3.80 (5, 3H).

### INT-53

### Di-tert-butyl 5-bromo-4-cyano-2-methoxyphenyldicarbamate

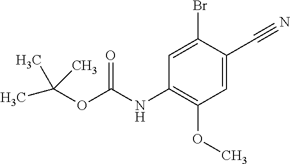 [SMILES: COc1cc(C#N)c(Br)cc1NC(=O)OC(C)(C)C]

To a stirred solution of aforementioned 4-amino-2-bromo-5-methoxybenzonitrile (INT-52, 25 g, 0.11 mol) in THF (500 mL) was added DIPEA (98 mL, 0.55 mol), di-tert-butyl dicarbonate (126 mL, 0.55 mol) and DMAP (6.73 g, 0.055 mol). The reaction mixture was stirred for 16 h at room temperature. After completion of the reaction, the reaction mixture was filtered over a Celite® pad. To the filtrate was added water (1 L) and the mixture was extracted with ethyl acetate (2×500 mL). The combined organic layers were concentrated to obtain the crude reaction product, which was triturated with pentane (200 mL) to give the title compound (25 g, 53%) as pale yellow solid. ¹H-NMR (400 MHz, DMSO-d₆) δ [ppm]: 7.41 (s, 1H), 7.14 (s, 1H), 3.86 (s, 3H), 1.42 (s, 18H).

### INT-54

### 4-Amino-5-methoxy-2-(o-tolyloxy)benzonitrile

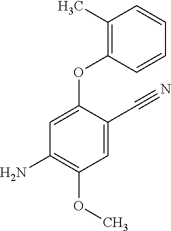 [SMILES: COc1cc(C#N)c(Oc2ccccc2C)cc1N]

To a stirred solution of di-tert-butyl 5-bromo-4-cyano-2-methoxyphenyldicarbamate (INT-53, 25 g, 0.058 mol) in DMF (250 mL) was added o-cresol (31.6 g, 0.292 mol), cesium carbonate (95.3 g, 0.292 mol) and copper powder (18.6 g, 0.292 mol). The reaction mixture was heated under reflux for 16 h. After completion of the reaction, the reaction mixture was cooled to room temperature, and filtered over a Celite® pad. The filtrate was added to ice water (1 L), and was extracted with ethyl acetate (2×500 mL). The combined organic layers were evaporated completely. The crude reaction product was purified by RP column chromatography using C₁₈ column using 70% ACN in water as an eluent to give the title compound (9.2 g, 62%) as yellow oil. ¹H-NMR (300 MHz, DMSO-d₆): δ [ppm]=7.35 (d, 1H), 7.28-7.10 (m, 2H), 7.07 (s, 1H), 6.96 (d, 1H), 6.00 (s, 1H), 5.92 (br s, 2H), 3.78 (s, 3H), 2.19 (s, 3H).

### INT-55

### 1-(4-Cyano-2-methoxy-5-(o-tolyloxy)phenyl)urea

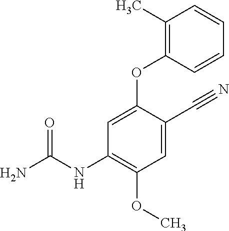 [SMILES: COc1cc(C#N)c(Oc2ccccc2C)cc1NC(N)=O]

To a stirred solution of 4-amino-5-methoxy-2-(o-tolyloxy) benzonitrile (INT-54, 10 g, 0.040 mol) in acetic acid (700 mL) and water (30 mL) was added sodium cyanate (6.40 g, 0.098 mol). The reaction mixture was at room temperature for 16 h. The reaction was monitored by thin layer chromatography. After completion of the reaction, the solid formed during reaction was filtered off, washed with petrol ether (200 mL) and dried under vacuum to give the title compound (10 g, 86%) as off-white solid. ¹H-NMR (300 MHz, DMSO-d₆): δ [ppm]=8.45 (s, 1H), 7.88 (s, 1H), 7.40 (s, 1H), 7.33 (br d, 1H), 7.25-7.17 (m, 1H), 7.15-7.07 (m, 1H), 6.85 (d, 1H), 6.47 (br s, 2H), 3.90 (s, 3H), 2.21 (s, 3H).

### INT-56

### 5-Methoxy-2-(o-tolyloxy)-4-(2,4,6-trioxotetrahydropyrimidin-1(2H)-yl)benzonitrile

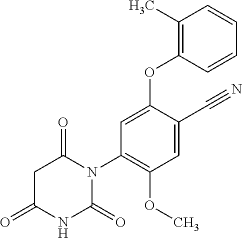 [SMILES: COc1cc(C#N)c(Oc2ccccc2C)cc1N1C(=O)CC(=O)NC1=O]

To stirred ethanol (200 mL) at 0° C. was added metallic sodium (6.2 g, 0.269 mol) and the mixture was stirred at 0° C. until the sodium metal was dissolved completely. Then was added the aforementioned 1-(4-cyano-2-methoxy-5-(o-tolyloxy) phenyl) urea (INT-55, 10 g, 0.033 mol) and diethyl malonate (27 g, 0.168 mol). The reaction mixture was stirred for 16 h at reflux temperature. After completion of the reaction, the solvent was evaporated under vacuum, and the thus obtained solid was acidified with 20% aq. HCl (up to 6.5-7 pH). The solid was filtered off and washed with water (50 mL) and dried under vacuum to give the title compound (6.8 g) as an off-white solid. ¹H-NMR (300 MHz, DMSO-d₆): δ [ppm]=11.56 (br s, 1H), 7.72 (5, 1H), 7.36 (br d, 1H), 7.32-7.23 (m, 1H), 7.23-7.14 (m, 1H), 7.01 (d, 1H), 6.81 (s, 1H), 3.80 (s, 3H), 3.36 (s, 2H), 2.16 (s, 3H).

### INT-57

### Ethyl (6-cyano-5-fluoropyridin-3-yl)carbamate

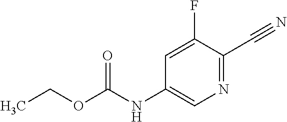

According to GP1, to a solution of commercially available 5-amino-3-fluoropyridine-2-carbonitrile (CAS: 573763-07-2, 1.0 g, 7.29 mmol) in pyridine (11 mL) was added ethyl carbonochloridate (840 μL, 8.8 mmol) and the resulting mixture was stirred at room temperature overnight. Upon completion of the reaction, an aq. solution of HCl (1 M) was slowly added and a precipitate was formed. The precipitate was collected by filtration to give the title compound (1.12 g, 70% yield, 50% purity) as a light yellow solid which was used without further purification in the next step.

¹H-NMR (400 MHz, DMSO-d₆) δ [ppm]: 1.251 (7.28), 1.268 (16.00), 1.286 (7.45), 4.175 (2.16), 4.193 (6.77), 4.211 (6.61), 4.228 (2.02), 8.051 (2.09), 8.056 (2.11), 8.080 (2.05), 8.085 (2.09), 8.520 (2.68), 8.523 (3.36), 8.528 (2.70), 10.744 (2.20); LC-MS (Method IV): Rₜ=0.93 min; ESIpos): m/z=209 [M+H]⁺.

### INT-58

### 2-Bromo-4-[2,6-dioxo-4-(trifluoromethyl)-3,6-dihydropyrimidin-1(2H)-yl]-5-methoxybenzonitrile

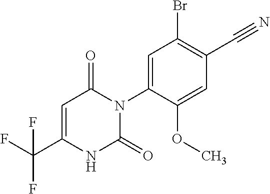 [SMILES: COc1cc(C#N)c(Br)cc1-n1c(=O)cc(C(F)(F)F)[nH]c1=O]

To anhydrous DMF (75 mL) was added 60% sodium hydride (6.0 g, 0.15 mol) at 0° C., and the mixture was stirred for 10 min. Subsequently, (Z)-ethyl 3-amino-4,4,4-trifluorobut-2-enoate (18.4 g, 0.10 mol) was added slowly at 0° C. and the mixture was stirred for 30 min. A solution of ethyl 5-bromo-4-cyano-2-methoxyphenylcarbamate (INT-27, 15 g, 0.05 mol) in DMF (75 mL) was added slowly. The reaction mixture was heated at reflux for 15 h. The reaction was monitored by thin layer chromatography. After completion of the reaction, the reaction mixture was cooled to room temperature and was poured into ice water, neutralised with 1M aqueous solution of hydrochloric acid and extracted twice with ethyl acetate (150 mL each). The organic layers were combined and washed with saturated brine solution (150 mL) and dried over anhydrous sodium sulfate, filtered and concentrated completely. The crude compound was purified by column chromatography on silica gel to give the title compound (6.3 g) as pale brown solid. ¹H-NMR (400 MHz, DMSO-d₆) δ [ppm]: 1.173 (0.40), 1.988 (0.77), 2.518 (1.62), 2.523 (1.10), 3.744 (0.43), 3.829 (16.00), 6.423 (3.50), 7.853 (6.07), 7.933 (7.21).

### EXPERIMENTAL SECTION—EXAMPLES

### Example 1

### 2-(2,6-Dimethylphenoxy)-4-[2,6-dioxo-4-(trifluoromethyl)-3,6-dihydropyrimidin-1 (2H)-yl]-5-fluorobenzonitrile

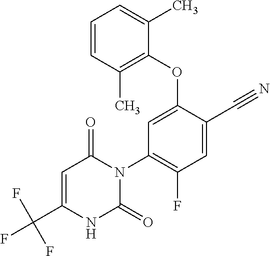 [SMILES: Cc1cccc(C)c1Oc1cc(-n2c(=O)cc(C(F)(F)F)[nH]c2=O)c(F)cc1C#N]

According to GP4, literature known 4-[2,6-dioxo-4-(trifluoromethyl)-3,6-dihydropyrimidin-1(2H)-yl]-2,5-difluorobenzonitrile (DE 10307142 A1 20040902, CAS: 162926-25-2, 100 mg, 315 μmol), commercially available 2,6-dimethylphenol (CAS: 576-26-1, 38.5 mg, 315 μmol) and potassium carbonate (109 mg, 788 μmol) were stirred in DMSO (2 mL) for 21 h. Upon reaction completion, the mixture was filtered and purified by HT HPLC giving the title compound as an ochre solid (29 mg; 21%). ¹H-NMR (400 MHz, DMSO-d₆) δ [ppm]: 2.090 (16.00), 2.518 (0.62), 2.523 (0.46), 3.352 (0.70), 6.339 (2.98), 6.840 (1.93), 6.855 (1.85), 7.143 (0.49), 7.158 (0.79), 7.166 (0.97), 7.180 (2.13), 7.204 (3.37), 7.219 (1.02), 7.221 (0.92), 7.225 (0.88), 8.152 (2.08), 8.174 (2.10); LC-MS (Method III): Rₜ=1.14 min; MS (ESIpos): m/z=419 [M+H]⁺.

### Example 2

### 2-(2-Chloro-6-methylphenoxy)-4-[2,6-dioxo-4-(trifluoromethyl)-3,6-dihydropyrimidin-1(2H)-yl]-5-fluorobenzonitrile

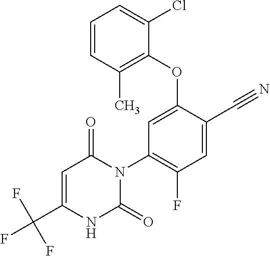 [SMILES: Cc1cccc(Cl)c1Oc1cc(-n2c(=O)cc(C(F)(F)F)[nH]c2=O)c(F)cc1C#N]

According to GP4, literature known 4-[2,6-dioxo-4-(trifluoromethyl)-3,6-dihydropyrimidin-1(2H)-yl]-2,5-difluorobenzonitrile (CAS: 162926-25-2, DE 10307142 A1 20040902, 150 mg, 473 μmol), commercially available 2-chloro-6-methylphenol (CAS: 87-64-9, 74.2 mg, 520 μmol) and potassium carbonate (163 mg, 1.18 mmol) were stirred in DMSO (1.6 mL) for 17 h. at 110° C. Upon reaction completion, the mixture was filtered and purified by HT HPLC to give the title compound as a beige solid (37.8 mg, 17%). ¹H-NMR (400 MHz, DMSO-d₆) δ [ppm]: 2.075 (16.00), 2.159 (15.28), 2.518 (4.00), 2.523 (2.51), 2.674 (0.54), 6.341 (3.09), 6.936 (2.21), 6.950 (2.21), 7.284 (1.71), 7.304 (4.18), 7.323 (3.00), 7.382 (2.17), 7.384 (2.46), 7.386 (2.07), 7.399 (1.29), 7.401 (1.57), 7.403 (1.45), 7.405 (1.29), 7.489 (2.11), 7.492 (2.08), 7.509 (1.81), 7.512 (1.70), 8.183 (3.74), 8.206 (3.69); LC-MS (Method I): Rₜ=1.21 min; MS (ESIpos): m/z=439 [M+H]⁺.

### Example 3

### 4-(4-Bromo-2,6-dioxo-3,6-dihydropyrimidin-1(2H)-yl)-5-fluoro-2-(2-methylphenoxy)benzonitrile

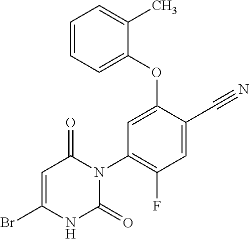 [SMILES: Cc1ccccc1Oc1cc(-n2c(=O)cc(Br)[nH]c2=O)c(F)cc1C#N]

Water (300 mg, 16.7 mmol, 300 μL, 1.25 eq) was added to a mixture of 5-fluoro-2-(2-methylphenoxy)-4-(2,4,6-trioxotetrahydropyrimidin-1(2H)-yl)benzonitrile (INT-48, 4.7 g, 13.3 mmol, 1.0 eq) in POBr₃ (38 g, 133 mmol, 13.5 mL, 9.96 eq) with stirring, and the mixed solution was stirred at 100° C. for 1 h. After reaction completion (TLC), the reaction mixture was poured into ice water (200 mL) and was extracted three times with ethyl acetate (100 mL). The obtained organic layer was dried over anhydrous sodium sulfate and then the solvent was distilled off under reduced pressure. The residue was purified by silica gel column chromatography (petroleum ether/ethyl acetate=5/1-1/1, product: Rₜ=0.5) to give the title compound (2.1 g, 36%) as a light yellow solid. ¹H-NMR (400 MHz, DMSO-d₆) δ [ppm]: 1.232 (0.44), 2.145 (16.00), 2.518 (3.35), 2.522 (2.00), 6.100 (2.99), 7.029 (2.32), 7.044 (2.28), 7.064 (1.96), 7.067 (2.02), 7.084 (2.34), 7.087 (2.32), 7.200 (0.81), 7.203 (0.86), 7.219 (2.24), 7.221 (2.14), 7.237 (1.67), 7.240 (1.48), 7.280 (1.13), 7.284 (1.29), 7.299 (1.58), 7.303 (1.69), 7.318 (0.67), 7.322 (0.63), 7.369 (1.85), 7.387 (1.51), 8.124 (3.54), 8.146 (3.46); LC-MS (Method IV): Rₜ=1.04 min; MS (ESIpos): m/z=416 [M+H]⁺.

### Example 4

### 4-[2,6-Dioxo-4-(trifluoromethyl)-3,6-dihydropyrimidin-1(2H)-yl]-5-methoxy-2-(2-methylphenoxy)benzonitrile (rac)

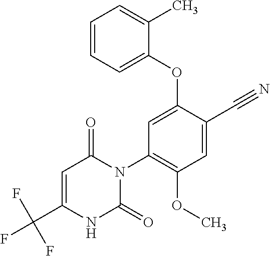 [SMILES: COc1cc(C#N)c(Oc2ccccc2C)cc1-n1c(=O)cc(C(F)(F)F)[nH]c1=O]

According to GP3, 2-bromo-4-[2,6-dioxo-4-(trifluoromethyl)-3,6-dihydropyrimidin-1(2H)-yl]-5-methoxybenzonitrile (INT-58, 600 mg, 1.54 mmol), commercially available 2-methylphenol (CAS: 95-48-7, 220 μL, 2.20 mmol), N,N-dimethylglycine (42.0 mg, 408 μmol), copper (I) iodide (39.5 mg, 208 μmol) and cesium carbonate (1.20 g, 3.69 mmol) were heated in DMF (30 mL) for 18 h at 140° C. Upon reaction completion, work-up and purification the title compound was obtained as an ochre solid (35 mg, 6%). ¹H-NMR (400 MHz, DMSO-d₆) δ [ppm]: 2.047 (0.66), 2.074 (0.85), 2.152 (0.76), 2.176 (12.89), 2.518 (1.41), 2.522 (0.91), 2.539 (0.76), 3.775 (1.56), 3.797 (16.00), 5.989 (0.59), 6.298 (3.49), 6.967 (1.81), 6.986 (7.77), 7.080 (0.50), 7.152 (0.85), 7.168 (1.81), 7.170 (1.83), 7.187 (1.25), 7.189 (1.20), 7.246 (1.01), 7.250 (1.04), 7.269 (1.41), 7.285 (0.57), 7.343 (1.64), 7.362 (1.29), 7.740 (6.02), 12.553 (0.41); LC-MS (Method I): Rₜ=1.15 min; MS (ESIneg): m/z=416 [M−H]⁻. Atropisomeric ratio: atrop 1/atrop 2=50:34 (16% impurities); Rₜ (atrop1)=3.20 min; Rt (atrop2)=4.99 min. The atropisomeric ratio was determined using the following chiral HPLC method: instrument: Agilent HPLC 1260; column: Chiralpak ID 3 μm 100×4.6 mm; eluent A: hexane+0.1 vol-% diethylamine (99%); eluent B: 2-propanol; gradient: 20-50% B in 7 min; flow: 1.4 mL/min; temperature: 25° C.; DAD 254 nm. The corresponding single atropisomers were separated using preparative chiral HPLC (Labomatic HD5000, Gilson GX-241, labcol Vario 4000; column: Chiralpak ID 5 μm 250×30 mm; eluent A: hexane+0.1 vol-% diethylamine (99%); eluent B: 2-propanol; isocratic 50% A+50% B; flow: 50 mL/min; UV 254 nm).

### Example 5

### 4-[2,6-Dioxo-4-(trifluoromethyl)-3,6-dihydropyrimidin-1(2H)-yl]-5-methoxy-2-(2-methylphenoxy)benzonitrile (atrop 1)

5 mg; colorless solid, [α]₀=+2.7°+/−0.92°.

### Example 6

### 4-[2,6-Dioxo-4-(trifluoromethyl)-3,6-dihydropyrimidin-1 (2H)-yl]-5-methoxy-2-(2-methylphenoxy)benzonitrile (atrop 2)

25 mg; colorless solid, [α]₀=−2.1°+/−0.47°.

### Example 7

### 4-[2,6-Dioxo-4-(trifluoromethyl)-3,6-dihydropyrimidin-1(2H)-yl]-5-methyl-2-(2-methylphenoxy)benzonitrile (rac)

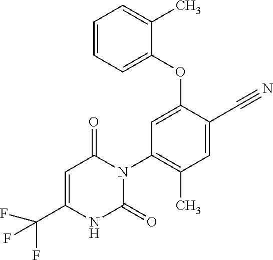 [SMILES: Cc1ccccc1Oc1cc(-n2c(=O)cc(C(F)(F)F)[nH]c2=O)c(C)cc1C#N]

According to GP4, 4-[2,6-Dioxo-4-(trifluoromethyl)-3,6-dihydropyrimidin-1(2H)-yl]-2-fluoro-5-methylbenzonitrile (INT-42, 100 mg, 319 μmol), potassium carbonate (110 mg, 798 μmol) and commercially available 2-methylphenol (CAS: 95-48-7, 33 μL, 320 μmol) were stirred in DMSO (2 mL) for 21 h. Upon reaction completion, work-up and purification the title compound was obtained as a light brown solid (18 mg, 13%). ¹H-NMR (400 MHz, DMSO-d₆) δ [ppm]: 2.035 (15.64), 2.135 (16.00), 2.518 (0.65), 2.523 (0.40), 6.304 (6.01), 6.879 (7.46), 7.050 (1.95), 7.053 (2.02), 7.070 (2.39), 7.073 (2.37), 7.183 (0.84), 7.185 (0.86), 7.201 (2.29), 7.204 (2.15), 7.219 (1.65), 7.222 (1.48), 7.269 (1.12), 7.272 (1.30), 7.287 (1.57), 7.291 (1.72), 7.307 (0.68), 7.310 (0.65), 7.353 (1.89), 7.355 (1.81), 7.372 (1.53), 7.920 (5.38); LC-MS (Method I): Rₜ=1.13 min; MS (ESIpos): m/z=402 [M+H]⁺. Atropisomeric ratio: atrop 1/atrop 2=44:44 (12% impurities); Rₜ (atrop1)=1.61 min; Rₜ (atrop2)=2.25 min. The atropisomeric ratio was determined using the following chiral HPLC method: instrument: Agilent HPLC 1260; column: cellulose SB 3 μm 100×4.6 mm; eluent A: hexane+0.1 vol-% TFA; eluent B: 2-propanol; isocratic: 80% A+20% B; flow 1.4 mL/min; T: 25° C.; DAD 254 nm. The corresponding single atropisomers were separated using preparative chiral HPLC (instrument: labomatic HD5000, Labocord-5000; Gilson GX-241, Labcol Vario 4000; column: cellulose SB 5 μm 250×30 mm; eluent A: hexane+0.1 vol % TFA; eluent B: 2-propanol; isocratic: 80% A+20% B; flow 50.0 mL/min; UV 254 nm).

### Example 8

### 4-[2,6-Dioxo-4-(trifluoromethyl)-3,6-dihydropyrimidin-1 (2H)-yl]-5-methyl-2-(2-methylphenoxy)benzonitrile (atrop 1)

4 mg; brown oil, contains TFA.

### Example 9

### 4-[2,6-Dioxo-4-(trifluoromethyl)-3,6-dihydropyrimidin-1(2H)-yl]-5-methyl-2-(2-methylphenoxy)benzonitrile (atrop 2)

3 mg; brown oil, contains TFA.

### Example 10

### 2-(2-Chlorophenoxy)-4-[2,6-dioxo-4-(trifluoromethyl)-3,6-dihydropyrimidin-1 (2H)-yl]-5-methylbenzonitrile (rac)

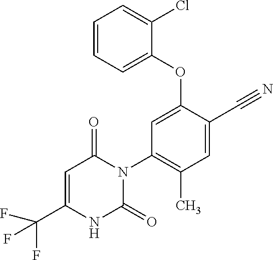 [SMILES: Cc1cc(C#N)c(Oc2ccccc2Cl)cc1-n1c(=O)cc(C(F)(F)F)[nH]c1=O]

According to GP4, 4-[2,6-Dioxo-4-(trifluoromethyl)-3,6-dihydropyrimidin-1(2H)-yl]-2-fluoro-5-methylbenzonitrile (INT-42, 70.0 mg, 50% purity, 112 μmol), potassium carbonate (38.6 mg, 279 μmol) and commercially available 2-chlorophenol (CAS: 95-57-8, 17.2 mg, 134 μmol) were stirred in DMSO (2 mL) for 3 d at 110° C. Upon reaction completion and purification the title compound was obtained as a brown solid (10 mg, 20%). ¹H-NMR (400 MHz, DMSO-d₆) δ [ppm]: 2.045 (16.00), 2.079 (0.71), 2.326 (0.52), 2.539 (4.17), 2.668 (0.48), 2.729 (0.95), 2.888 (1.09), 6.290 (3.52), 6.961 (5.08), 7.292 (1.90), 7.295 (2.27), 7.315 (3.71), 7.332 (2.47), 7.336 (2.05), 7.352 (1.65), 7.355 (1.38), 7.431 (1.60), 7.435 (1.70), 7.451 (2.18), 7.455 (2.15), 7.470 (0.95), 7.474 (0.94), 7.640 (2.56), 7.643 (2.55), 7.660 (2.35), 7.663 (2.16), 7.950 (5.80); LC-MS (Method I): Rₜ=1.11 min; MS (ESIneg): m/z=420 [M−H]⁻.

### Example 11

### 5-Chloro-4-[2,6-dioxo-4-(trifluoromethyl)-3,6-dihydropyrimidin-1(2H)-yl]-2-(2-methylphenoxy)benzonitrile (rac)

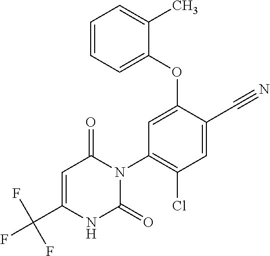 [SMILES: Cc1ccccc1Oc1cc(-n2c(=O)cc(C(F)(F)F)[nH]c2=O)c(Cl)cc1C#N]

According to GP2, to a solution of sodium hydride (60% in oil, 152 mg, 3.79 mmol) in DMF (50 mL), commercially available ethyl 3-amino-4,4,4-trifluorobut-2-enoate (CAS: 372-29-2, 690 mg, 3.79 mmol) and methyl [2-chloro-4-cyano-5-(2-methylphenoxy)phenyl]carbamate (INT-32, 1.00 g, 80% purity, 2.53 mmol) were added, and the mixture was at 100° C. overnight. Upon completion of the reaction, aq. ammonium chloride solution was added at 0° C. to stop the reaction. The solvent was removed in vacuo and the residue was purified by Prep-HPLC [column: XBridge C₁₈ 19*150; eluent A: water/0.1% TFA, eluent B: ACN flow rate: 20 mL/min; gradient: 40% B to 60% B in 8 min, hold 1.5 min; 254 nm Rₜ=7.80 min] to give the title compound (480 mg, 44%) as an off-white solid. ¹H-NMR (400 MHz, DMSO-d₆): δ [ppm]=2.13 (s, 3H), 6.37 (s, 1H), 7.10 (s, 1H), 7.16 (d, 1H), 7.26 (t, 1H), 7.32 (t, 1H), 7.39 (d, 1H), 8.35 (s, 1H), 12.68 (br, 1H). LC-MS (Method E, 0-3.00 min 5-95% B): Rₜ=1.41 min; MS (ESIneg): m/z=420 (M−H)⁻. The corresponding single atropisomers were separated by chiral HPLC [column: CHIRAL ART Cellulose-SB, 2×25 cm, 5 μm; eluent A: n-Hexane (0.1% TFA), eluent B: IPA; Gradient: 20% B to 20% B in 16 min).

### Example 12

### 5-Chloro-4-[2,6-dioxo-4-(trifluoromethyl)-3,6-dihydropyrimidin-1(2H)-yl]-2-(2-methylphenoxy)benzonitrile (atrop 1)

Rₜ=7.55 min, 161.3 mg; light yellow solid

### Example 13

### 5-Chloro-4-[2,6-dioxo-4-(trifluoromethyl)-3,6-dihydropyrimidin-1(2H)-yl]-2-(2-methylphenoxy)benzonitrile (atrop 2)

Rₜ=9.20 min, 142 mg; light yellow solid

### Example 14

### 4-[2,6-Dioxo-4-(trifluoromethyl)-3,6-dihydropyrimidin-1(2H)-yl]-5-iodo-2-(2-methylphenoxy)benzonitrile (rac)

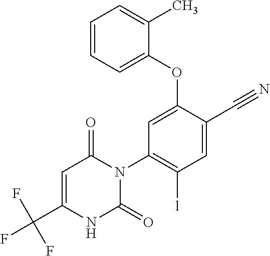

According to GP2, to a slurry of sodium hydride (47 mg, 1.2 mmol, 60% in mineral oil), in DMF (5 mL) were added commercially available ethyl 3-amino-4,4,4-trifluorobut-2-enoate (CAS: 372-29-2, 215 mg, 1.2 mmol), and methyl 4-cyano-2-iodo-5-(o-tolyloxy)phenylcarbamate (INT-33, 300 mg, 0.6 mmol), at 0° C. The resulting mixture was stirred at 100° C. overnight under nitrogen atmosphere. Upon completion of the reaction, aq. ammonium chloride solution was added at 0° C. and the resulting mixture was extracted with ethyl acetate. The combined organic layers were dried over anhydrous sodium sulfate and concentrated in vacuo. The residue was purified by Prep-HPLC [column: eluent A: water (0.1% TFA), eluent B: ACN; gradient: 40% B to 60% B in 8 min] to give 113.2 mg (37% yield) of the title compound as a white solid. ¹H-NMR (400 MHz, DMSO-d₆): δ [ppm]=2.12 (s, 3H), 6.33 (s, 1H), 6.96 (d, 1H), 7.14 (d, 1H), 7.21-7.26 (m, 1H), 7.29-7.34 (m, 1H), 7.38 (d, 1H), 8.50 (s, 1H), 12.66 (br, 1H); LC-MS (Method E, 0-3.00 min 5-95% B): Rₜ=1.41 min; MS (ESIneg): m/z=512 (M−H)⁻.

### Example 15

### 2-(2-Chlorophenoxy)-4-[2,6-dioxo-4-(trifluoromethyl)-3,6-dihydropyrimidin-1(2H)-yl]-5-methoxybenzonitrile (rac)

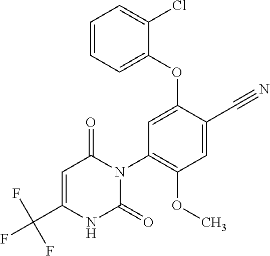 [SMILES: COc1cc(C#N)c(Oc2ccccc2Cl)cc1-n1c(=O)cc(C(F)(F)F)[nH]c1=O]

According to GP3, INT-48 (500 mg, 1.28 mmol), commercially available 2-chlorophenol (CAS: 95-57-8, 234 mg, 1.82 mmol), N,N-dimethylglycine (35.0 mg, 340 μmol), copper (i) iodide (33.0 mg, 173 μmol) and cesium carbonate (1.00 g, 3.08 mmol) were heated in DMF (25 mL) for 18 h at 140° C. Upon reaction completion, work-up and purification the title compound was obtained as a brown solid (35 mg, 6%). ¹H-NMR (400 MHz, DMSO-d₆) δ [ppm]: 2.074 (9.53), 2.518 (1.80), 2.523 (1.21), 3.808 (16.00), 6.300 (1.97), 6.873 (0.51), 7.079 (4.74), 7.199 (1.70), 7.203 (1.89), 7.219 (2.01), 7.223 (2.13), 7.280 (1.04), 7.284 (1.03), 7.299 (1.77), 7.303 (1.61), 7.318 (1.52), 7.322 (1.29), 7.410 (1.45), 7.414 (1.41), 7.429 (1.45), 7.433 (1.48), 7.450 (0.94), 7.453 (0.92), 7.631 (2.25), 7.634 (2.24), 7.650 (2.05), 7.654 (1.85), 7.772 (5.87), 9.945 (0.58), 10.094 (0.47); LC-MS (Method IV): Rₜ=1.15 min; MS (ESIpos): m/z=437 [M+H]⁺. Atropisomeric ratio: atrop 1/atrop 2=52:48; Rₜ (atrop1)=1.77 min; Rₜ (atrop2)=4.74 min. The atropisomeric ratio was determined using the following chiral HPLC method: Instrument: Agilent: 1260, Aurora SFC-Modul; column: Chiralpak ID 5 μm 100×4.6 mm; eluent A: CO₂, eluent B: 2-propanol+0.2 vol-% diethylamine (99%); isocratic: 21% B; flow: 4.0 mL/min; T: 37.5° C.; BPR: 100 bar; MWD: 220 nm. The corresponding single atropisomers were separated using preparative chiral HPLC: instrument: Sepiatec: Prep SFC100; column: chiralpak ID 5 μm 250×30 mm; eluent A: CO₂, eluent B: 2-propanol+0.4 vol-% diethylamine (99%); isocratic: 21% B; flow 100.0 mL/min, T: 40° C.; BPR: 150 bar; MWD: 220 nm.

### Example 16

### 2-(2-Chlorophenoxy)-4-[2,6-dioxo-4-(trifluoromethyl)-3,6-dihydropyrimidin-1(2H)-yl]-5-methoxybenzonitrile (atrop 1)

11.2 mg; brown solid.

### Example 17

### 2-(2-Chlorophenoxy)-4-[2,6-dioxo-4-(trifluoromethyl)-3,6-dihydropyrimidin-1(2H)-yl]-5-methoxybenzonitrile (atrop 2)

16 mg; brown solid.

### Example 18

### 5-Bromo-4-[2,6-dioxo-4-(trifluoromethyl)-3,6-dihydropyrimidin-1(2H)-yl]-2-(2-methylphenoxy)benzonitrile (rac)

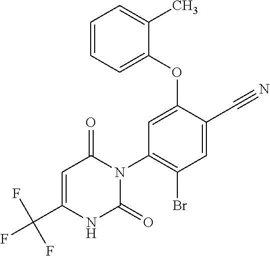 [SMILES: Cc1ccccc1Oc1cc(-n2c(=O)cc(C(F)(F)F)[nH]c2=O)c(Br)cc1C#N]

According to GP2, to a suspension of sodium hydride (337 mg, 8.4 mmol, 60% in mineral oil) in DMF (50 mL) was added commercially available ethyl 3-amino-4,4,4-trifluorobut-2-enoate (CAS: 372-29-2, 1.5 g, 8.4 mmol) at 0° C. After stirring at this temperature for 30 min, methyl [2-bromo-4-cyano-5-(2-methylphenoxy)phenyl]carbamate (INT-31, 1.9 g, 4.2 mmol, 80% purity) was added to the above slurry. The resulting mixture was stirred at 100° C. overnight under nitrogen atmosphere. After cooling to room temperature, saturated aq. ammonium chloride solution was added at 0° C. and the resulting mixture was extracted with ethyl acetate. The combined organic layer was dried over anhydrous sodium sulfate and was concentrated in vacuo. The residue was purified by silica gel column chromatography (petroleum ether:ethyl acetate=1:1) to give (1.1 g, 50% yield) of the title compound as a white solid. ¹H-NMR (400 MHz, DMSO-d₆): δ [ppm]=2.13 (s, 3H), 6.21 (br, 1H), 6.96 (s, 1H), 7.15 (d, 1H), 7.21-7.26 (m, 1H), 7.30-7.34 (m, 1H), 7.39 (d, 1H), 8.42 (s, 1H), 12.68 (br, 1H). LC-MS (Method E, 0-3.00 min 5-95% B): Rₜ=1.39 min; MS (ESIneg): m/z=464 (M−H)⁻.

### Example 19

### 4-[4-Bromo-2,6-dioxo-3,6-dihydropyrimidin-1(2H)-yl]-5-methoxy-2-(2-methylphenoxy)benzonitrile (rac)

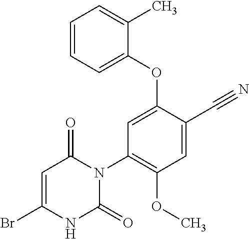 [SMILES: COc1cc(C#N)c(Oc2ccccc2C)cc1-n1c(=O)cc(Br)[nH]c1=O]

To a stirred solution of 5-fluoro-2-(2-methylphenoxy)-4-(2,4,6-trioxotetrahydropyrimidin-1(2H)-yl)benzonitrile (INT-56, 6.5 g, 0.017 mol) in acetonitrile (65 mL) was added phosphoryl bromide (POBr₃, 12.8 g, 0.045 mol). The resulting mixture was stirred at 110° C. under microwave irradiation for 30 min. After completion of the reaction, the solvent was removed under reduced pressure. The crude residue was treated with saturated aq. solution of sodium bicarbonate (500 mL), and the mixture was then extracted twice with ethyl acetate (250 mL each). The combined organic layers were concentrated under vacuum. The residue was purified using RP C₁₈ 80 g column, eluting 40% acetonitrile in water to give the title compound (3.02 g, 39%) as an off-white solid.

¹H-NMR (400 MHz, DMSO-d₆) δ [ppm]: 1.233 (0.70), 2.074 (4.12), 2.183 (12.55), 2.518 (1.30), 2.522 (0.80), 3.742 (0.42), 3.757 (16.00), 5.683 (0.46), 6.724 (1.13), 6.907 (1.60), 6.910 (1.68), 6.927 (1.82), 6.929 (1.82), 7.125 (0.72), 7.129 (0.77), 7.144 (1.83), 7.147 (1.83), 7.163 (1.25), 7.165 (1.17), 7.225 (0.87), 7.228 (0.99), 7.244 (1.29), 7.248 (1.38), 7.264 (0.59), 7.267 (0.58), 7.329 (1.49), 7.331 (1.45), 7.347 (1.26), 7.640 (4.70). LC-MS (Method I): Rₜ=1.01 min; MS (ESIpos): m/z=428 (M−H)⁺. Atropisomeric ratio: atrop 1/atrop 2=49:49; Rₜ (atrop1)=2.10 min; Rₜ (atrop2)=2.59 min. The atropisomeric ratio was determined using the following chiral HPLC method: Instrument: Waters Alliance 2695A gilent HPLC 1260; column: Chiralpak IA 3 μm 100×4.6 mm; eluent A: hexane+0.1 vol-% TFA (99%), eluent B: 2-propanol; isocratic: 20% B; flow: 1.4 mL/min; T: 25° C.; DAD 254 nm. The corresponding single atropisomers were separated using preparative chiral HPLC: instrument: Labomatic HD5000, Labocord-5000; Gilson GX-241, Labcol Vario 4000; column: Chiralpak IA 5 μm 250×30 mm; eluent A: hexane+0.1 vol-% TFA (99%); eluent B: 2-propanol; isocratic: 80% A+20% B; flow 60.0 mL/min; UV 254 nm.

### Example 20

### 4-[4-Bromo-2,6-dioxo-3,6-dihydropyrimidin-1(2H)-yl]-5-methoxy-2-(2-methylphenoxy)benzonitrile (atrop 1)

25 mg; white solid.

### Example 21

### 4-[4-Bromo-2,6-dioxo-3,6-dihydropyrimidin-1(2H)-yl]-5-methoxy-2-(2-methylphenoxy)benzonitrile (atrop 2)

20 mg; white solid.

### Example 22

### 4-[4-(Difluoromethyl)-2,6-dioxo-3,6-dihydropyrimidin-1(2H)-yl]-5-methoxy-2-(2-methylphenoxy)benzonitrile (rac)

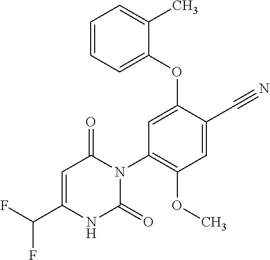 [SMILES: COc1cc(C#N)c(Oc2ccccc2C)cc1-n1c(=O)cc(C(F)F)[nH]c1=O]

According to GP4, 4-[4-(difluoromethyl)-2,6-dioxo-3,6-dihydropyrimidin-1(2H)-yl]-2-fluoro-5-methoxybenzonitrile (INT-39, (20.0 mg, 64.3 μmol), commercially available 2-methylphenol (8.0 μL, 77 μmol) and potassium carbonate (22.2 mg, 161 μmol) were stirred in DMSO (850 μL) for 3 days at 110° C. Upon reaction completion, the mixture was filtered and purified by HT HPLC giving the title compound as an ochre solid (1 mg; 3%). ¹H-NMR (400 MHz, METHANOL-d₄) δ [ppm]: 1.192 (2.23), 1.983 (0.45), 2.124 (1.82), 2.209 (1.66), 2.251 (10.49), 3.211 (0.71), 3.847 (16.00), 5.986 (2.65), 6.454 (0.96), 6.588 (1.96), 6.721 (0.91), 6.775 (4.46), 6.951 (1.18), 6.972 (1.35), 7.104 (0.47), 7.122 (1.22), 7.141 (0.85), 7.197 (0.63), 7.216 (0.86), 7.288 (0.98), 7.306 (0.81), 7.542 (4.64); LC-MS (Method IV): Rₜ=1.04 min; MS (ESIpos): m/z=399 [M+H]⁺.

### Example 23

### 4-(4-Cyclopropyl-2,6-dioxo-3,6-dihydropyrimidin-1(2H)-yl)-5-methoxy-2-(2-methylphenoxy)benzonitrile (rac)

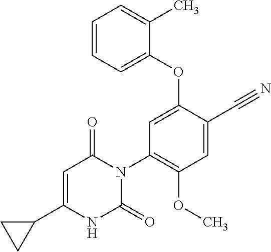 [SMILES: COc1cc(C#N)c(Oc2ccccc2C)cc1-n1c(=O)cc(C2CC2)[nH]c1=O]

In a pressure vessel, the aforementioned 4-[4-bromo-2,6-dioxo-3,6-dihydropyrimidin-1(2H)-yl]-5-methoxy-2-(2-methylphenoxy)benzonitrile (Example 19, 250 mg, 584 μmol), the commercially available catalyst palladium(ii) acetate (CAS: 3375-31-3, 6.92 mg, 11.7 μmol), sodium carbonate (161 mg, 1.17 mmol), commercially available potassium cyclopropyl(trifluoro)borate (CAS: 1065010-87-8, 95.0 mg, 642 μmol) and the commercially available tricyclohexylphosphine (CAS: 2622-14-2, 6.55 mg, 23.4 μmol) were suspended in toluene (4.9 mL) under argon atmosphere. The reaction mixture was heated to 90° C. for 18 h. After cooling, the mixture was filtered and washed with ethyl acetate. The residue was purified by HPLC giving the target compound as a light yellow solid (5 mg, 2%, 85% purity). ¹H-NMR (400 MHz, DMSO-d₆) δ [ppm]: 0.899 (0.40), 0.993 (0.73), 0.998 (0.59), 1.013 (0.71), 1.019 (0.64), 1.648 (0.40), 1.656 (0.43), 1.669 (0.52), 2.181 (11.79), 2.190 (5.48), 2.322 (0.68), 2.327 (0.92), 2.331 (0.69), 2.522 (2.27), 2.539 (4.00), 2.664 (0.68), 2.669 (0.94), 2.673 (0.68), 3.776 (16.00), 5.280 (1.14), 5.284 (1.16), 6.867 (2.84), 6.886 (0.73), 6.901 (1.02), 6.920 (2.27), 6.939 (1.75), 7.134 (0.75), 7.152 (1.80), 7.170 (1.30), 7.234 (0.99), 7.252 (1.42), 7.269 (0.64), 7.332 (2.10), 7.351 (1.77), 7.693 (5.76), 11.292 (0.73); LC-MS (Method IV): Rₜ=1.04 min; MS (ESIpos): m/z=389 [M+H]⁺.

### Example 24

### 4-{4-[Difluoro(phenyl)methyl]-2,6-dioxo-3,6-dihydropyrimidin-1(2H)-yl}-5-methoxy-2-(2-methylphenoxy)benzonitrile (rac)

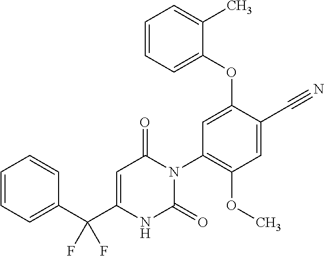 [SMILES: COc1cc(C#N)c(Oc2ccccc2C)cc1-n1c(=O)cc(C(F)(F)c2ccccc2)[nH]c1=O]

According to GP3, 2-bromo-4-{4-[difluoro(phenyl)methyl]-2,6-dioxo-3,6-dihydropyrimidin-1(2H)-yl}-5-methoxybenzonitrile (INT-38, 60.0 mg, 134 μmol), commercially available 2-methylphenol (CAS: 95-48-7, 17 μL, 160 μmol), N,N-dimethylglycine (3.66 mg, 35.5 μmol), copper (i) iodide (3.44 mg, 18.1 μmol) and cesium carbonate (105 mg, 321 μmol) were heated in DMF for 18 h at 140° C. Upon reaction completion, work-up and purification the target compound was obtained as a brown solid (6 mg, 8%). ¹H-NMR (400 MHz, DMSO-d₆) δ [ppm]: 2.083 (0.68), 2.176 (12.77), 2.205 (0.70), 2.226 (2.08), 2.518 (1.77), 2.523 (1.13), 3.779 (16.00), 3.848 (0.41), 5.978 (2.85), 6.868 (0.47), 6.900 (0.64), 6.921 (0.67), 6.934 (1.68), 6.951 (1.85), 6.954 (1.85), 7.013 (2.75), 7.131 (0.71), 7.135 (0.77), 7.150 (1.78), 7.153 (1.77), 7.169 (1.20), 7.171 (1.16), 7.227 (0.83), 7.230 (0.99), 7.246 (1.24), 7.250 (1.39), 7.266 (0.54), 7.269 (0.53), 7.328 (1.44), 7.330 (1.41), 7.347 (1.40), 7.421 (0.43), 7.506 (0.41), 7.540 (0.71), 7.556 (2.46), 7.575 (2.84), 7.584 (1.74), 7.602 (1.18), 7.691 (2.29), 7.695 (2.81), 7.714 (7.67), 8.429 (0.72), 10.367 (0.90), 11.433 (1.10), 12.024 (1.57); LC-MS (Method IV): Rₜ=1.23 min; MS (ESIpos): m/z=475 [M+H]⁺.

### Example 25

### 4-{4-[Chloro(difluoro)methyl]-2,6-dioxo-3,6-dihydropyrimidin-1(2H)-yl}-5-methoxy-2-(2-methylphenoxy)benzonitrile (rac)

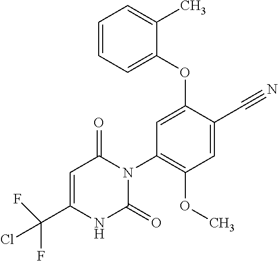 [SMILES: COc1cc(C#N)c(Oc2ccccc2C)cc1-n1c(=O)cc(C(F)(F)Cl)[nH]c1=O]

According to GP4, 4-{4-[chloro(difluoro)methyl]-2,6-dioxo-3,6-dihydropyrimidin-1(2H)-yl}-2-fluoro-5-methoxybenzonitrile (INT-40, 120 mg, 70% purity, 243 μmol), commercially available 2-methylphenol (31.5 mg, 292 μmol) and potassium carbonate (84.0 mg, 608 μmol) were stirred in DMSO (3.2 mL) for 21 h at 110° C. An additional equivalent of 2-methylphenol was added and the mixture was stirred for additional 18 h. Upon reaction completion, the mixture was filtered and purified by HT HPLC giving the title compound as an ochre solid (5 mg; 4%). ¹H-NMR (400 MHz, DMSO-d₆) δ [ppm]: 0.852 (0.44), 1.234 (1.07), 2.084 (0.50), 2.170 (1.39), 2.180 (13.42), 2.332 (2.65), 2.518 (16.00), 2.522 (10.08), 2.539 (1.07), 2.673 (2.65), 3.774 (1.45), 3.799 (15.75), 5.988 (0.57), 6.187 (0.94), 6.959 (1.70), 6.979 (2.08), 7.010 (1.51), 7.080 (0.50), 7.148 (0.76), 7.167 (1.89), 7.183 (1.32), 7.247 (1.13), 7.266 (1.51), 7.285 (0.63), 7.343 (1.64), 7.361 (1.39), 7.735 (4.60), 12.501 (0.44); LC-MS (Method IV): Rₜ=1.09 min; MS (ESIpos): m/z=434 [M+H]⁺.

### Example 26

### 5-Chloro-4-{4-[chloro(difluoro)methyl]-2,6-dioxo-3,6-dihydropyrimidin-1(2H)-yl}-2-(2-methylphenoxy)benzonitrile (rac)

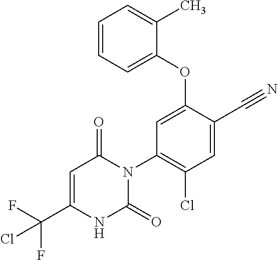 [SMILES: Cc1ccccc1Oc1cc(-n2c(=O)cc(C(F)(F)Cl)[nH]c2=O)c(Cl)cc1C#N]

According to GP2, to a suspension of sodium hydride (27 mg, 0.68 mmol, 60% in mineral oil) in DMF (3 mL) were added methyl [2-chloro-4-cyano-5-(2-methylphenoxy)phenyl]carbamate (INT-32, 150 mg, 0.90 mmol) and ethyl 3-amino-4-chloro-4,4-difluorobut-2-enoate (153 mg, 0.45 mmol, 70% purity) at 0° C. The resulting mixture was then heated to 100° C. and stirred at this temperature overnight under nitrogen atmosphere. After cooling to room temperature, aq. ammonium chloride solution was added at 0° C. and the solvent was removed in vacuo. The residue was diluted with water and the resulting mixture was extracted with ethyl acetate. The combined organic layer was dried over anhydrous sodium sulfate and concentrated in vacuo. The residue was purified by Prep-HPLC [column: Gemini C₁₈ 30×100 mm; eluent A: water (0.1% ammoniac), eluent B: Acetonitrile; Gradient: 10% B to 42% B in 8 min] to give the title compound (10.9 mg (5% yield) as an off-white solid. 1H-NMR (400 MHz, DMSO-d₆): δ [ppm]=2.08 (s, 3H), 6.24 (5, 1H), 6.95 (s, 1H), 7.15 (d, 1H), 7.25 (t, 1H), 7.33 (t, 1H), 7.39 (d, 1H), 8.35 (s, 1H), 12.66 (br, 1H); LC-MS (Method E, 0-1.80 min 5-100% B): Rt=1.05 min; MS (ESIneg): m/z=436 (M−H)⁻.

### Example 27

### 3-[4-Chloro-2-methoxy-5-(2-methylphenoxy)phenyl]-6-(trifluoromethyl)pyrimidine-2,4(1H,3H)-dione (rac)

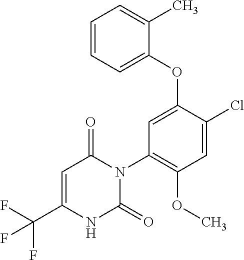 [SMILES: COc1cc(Cl)c(Oc2ccccc2C)cc1-n1c(=O)cc(C(F)(F)F)[nH]c1=O]

According to GP3, 3-(5-bromo-4-chloro-2-methoxyphenyl)-6-(trifluoromethyl)pyrimidine-2,4(1H,3H)-dione (INT-45, 160 mg, 400 μmol), commercially available 2-methylphenol (CAS: 95-48-7, 59 μL, 570 μmol), N,N-dimethylglycine (10.9 mg, 106 μmol), copper (i) iodide (10.3 mg, 54.1 μmol) and cesium carbonate (313 mg, 961 μmol) were heated in DMF (7.9 mL) for 18 h at 140° C. Upon reaction completion, work-up and purification the title compound was obtained as a dark brown solid (16 mg, 8%). ¹H-NMR (400 MHz, DMSO-d₆) δ [ppm]: 2.168 (1.32), 2.226 (0.64), 2.237 (11.82), 2.518 (1.52), 2.523 (1.04), 3.782 (16.00), 6.300 (2.24), 6.670 (1.60), 6.672 (1.67), 6.690 (1.75), 6.692 (1.74), 7.026 (0.73), 7.028 (0.76), 7.045 (1.72), 7.047 (1.74), 7.063 (1.33), 7.070 (5.20), 7.154 (0.79), 7.156 (0.83), 7.172 (1.20), 7.176 (1.28), 7.192 (0.57), 7.195 (0.56), 7.286 (1.32), 7.288 (1.30), 7.304 (1.16), 7.429 (6.03), 8.428 (0.55), 12.530 (0.48); LC-MS (Method I): Rₜ=1.22 min; MS (ESIpos): m/z=427 [M+H]⁺.

### Example 28

### 3-[4-Chloro-2-methoxy-5-(2-methylphenoxy)phenyl]-6-(difluoromethyl)pyrimidine-2,4(1H,3H)-dione (rac)

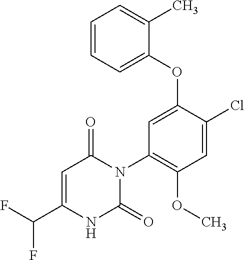 [SMILES: COc1cc(Cl)c(Oc2ccccc2C)cc1-n1c(=O)cc(C(F)F)[nH]c1=O]

According to GP3, 3-(5-bromo-4-chloro-2-methoxyphenyl)-6-(difluoromethyl)pyrimidine-2,4(1H,3H)-dione (INT-41, 35.0 mg, 91.7 μmol), commercially available 2-methylphenol (CAS: 95-48-7, 14.1 mg, 130 μmol), N,N-dimethylglycine (2.51 mg, 24.3 μmol), copper (i) iodide (32.36 mg, 12.4 μmol) and cesium carbonate (71.7 mg, 220 μmol) were heated in DMF (1.8 mL) for 18 h at 140° C. Upon reaction completion, work-up and purification the title compound was obtained as a dark brown solid (2 mg, 5%). ¹H-NMR (400 MHz, DMSO-d₆) δ [ppm]: 1.999 (0.48), 2.160 (1.20), 2.246 (12.25), 2.518 (5.00), 2.522 (3.06), 2.673 (0.89), 3.775 (16.00), 3.808 (0.61), 5.992 (3.04), 6.637 (1.81), 6.655 (2.80), 6.787 (2.13), 6.920 (0.89), 7.015 (0.84), 7.030 (1.79), 7.033 (1.79), 7.049 (1.18), 7.078 (6.83), 7.145 (0.86), 7.165 (1.33), 7.181 (0.61), 7.279 (1.41), 7.297 (1.24), 7.416 (6.16), 12.000 (0.89); LC-MS (Method IV): Rₜ=1.13 min; MS (ESIpos): m/z=409 [M+H]⁺.

### Example 29

### 3-[4-Chloro-3-(2-methylphenoxy)naphthalen-1-yl]-6-[difluoro(phenyl)methyl]pyrimidine-2,4(1H,3H)-dione (rac)

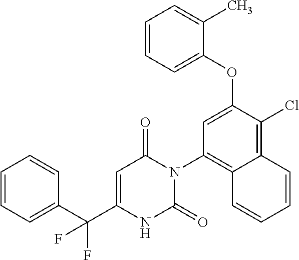 [SMILES: Cc1ccccc1Oc1cc(-n2c(=O)cc(C(F)(F)c3ccccc3)[nH]c2=O)c2ccccc2c1Cl]

According to GP2, to a suspension of sodium hydride (60% in mineral oil) (1.17 g, 29.32 mmol) in DMF (40 mL) was added a solution of ethyl 3-amino-4,4-difluoro-4-phenylbut-2-enoate (INT-3, 4.24 g, 17.59 mmol) in DMF (10 mL) at 0° C., and stirred for 10 min. Then was slowly added a solution of methyl [4-chloro-3-(2-methylphenoxy)naphthalen-1-yl]carbamate (INT-34, 5.0 g, 14.66 mmol) in DMF (10 mL). The resulting mixture was allowed to warm up to room temperature for 10 min and was then heated at 100° C. for 24 h under nitrogen atmosphere. Upon completion of the reaction, the reaction mixture was cooled to 0° C., and saturated aqueous ammonium chloride solution was added at 0° C. The solvent was removed in vacuo and the residue was diluted with water and the mixture was extracted with ethyl acetate and the combined organic layers were dried over anhydrous sodium sulfate, filtered and the solvent was removed in vacuo. The residue was purified by Prep-HPLC to afford the tite compound (2.1 g, 14%) as an off-white solid. ¹H-NMR (400 MHz, DMSO-d₆) δ [ppm]: 2.249 (16.00), 2.518 (4.29), 2.523 (2.61), 6.043 (1.94), 6.791 (2.09), 6.793 (2.19), 6.810 (2.32), 6.813 (2.32), 7.086 (0.92), 7.089 (0.95), 7.105 (2.34), 7.108 (2.27), 7.123 (1.56), 7.126 (1.50), 7.180 (1.09), 7.183 (1.20), 7.199 (1.63), 7.203 (1.73), 7.219 (0.71), 7.222 (0.71), 7.334 (1.81), 7.352 (1.60), 7.403 (1.18), 7.561 (1.73), 7.563 (1.81), 7.580 (4.28), 7.599 (5.16), 7.621 (1.28), 7.748 (3.32), 7.752 (3.24), 7.757 (3.37), 7.769 (4.03), 7.772 (3.86), 7.786 (1.51), 8.286 (2.66), 8.306 (2.22), 12.092 (2.43); LC-MS (Method IV): Rₜ=1.44 min; MS (ESIpos): m/z=505 [M+H]⁺. The corresponding single atropisomers were separated using preparative chiral HPLC: instrument: Sepiatec: Prep SFC100; column: Chiralpak IG 5 μm 250×30 mm; eluent A: CO₂; eluent B: methanol; isocratic: 20% B; flow: 100 mL/min; temperature: 40° C.; BPR: 150 bar; UV: 254 nm;

### Example 30

### 3-[4-chloro-3-(2-methylphenoxy)naphthalen-1-yl]-6-[difluoro(phenyl)methyl]pyrimidine-2,4(1H,3H)-dione (atrop 1)

¹H-NMR (400 MHz, DMSO-d₆) δ [ppm]: 1.107 (3.88), 2.249 (2.23), 2.518 (0.69), 2.523 (0.46), 2.539 (16.00), 7.578 (0.55), 7.597 (0.64), 7.750 (0.48), 7.768 (0.55); LC-MS (Method IV): Rₜ=1.44 min; MS (ESIpos): m/z=505 [M+H]⁺; optical rotation [α]: −1.0°+1-0.55°; honey-like sticky oil.

### Example 31

### 3-[4-chloro-3-(2-methylphenoxy)naphthalen-1-yl]-6-[difluoro(phenyl)methyl]pyrimidine-2,4(1H,3H)-dione (atrop 2)

¹H-NMR (400 MHz, DMSO-d₆) δ [ppm]: 2:25 (s, 3H), 6.04 (bs, 1H), 6.80 (dd, 1H), 7.11, 7.20 (2 td, 1H each), 7.34 (dd, 1H), 7.41 (bs, 1H), 7.56-7.63 (m, 4H), 7.75-7.79 (m, 4H), 8.30 (dd, 1H), 12.09 (bs, 1H); LC-MS (Method IV): Rₜ=1.44 min; MS (ESIpos): m/z=505 [M+H]⁺; optical rotation [α]: +4.7°+1-0.68°; Off-white solid.

### Example 32

### 3-[4-Chloro-3-(2-methylphenoxy)naphthalen-1-0]-6-(trifluoromethyl)pyrimidine-2,4(1H,3H)-dione (rac)

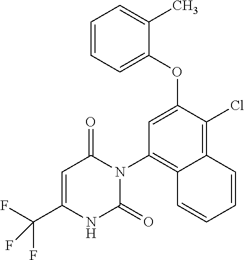 [SMILES: Cc1ccccc1Oc1cc(-n2c(=O)cc(C(F)(F)F)[nH]c2=O)c2ccccc2c1Cl]

According to GP2, to a suspension of sodium hydride (60% in mineral oil) (1.17 g, 29.3 mmol) in DMF (40 mL) was added a solution of ethyl 3-amino-4,4,4-trifluorobut-2-enoate (5.36 g, 29.3 mmol) in DMF (10 mL) at 0° C. and the mixture was stirred for 10 min. Then was slowly added a solution of methyl [4-chloro-3-(2-methylphenoxy)naphthalen-1-yl]carbamate (INT-34, 5.0 g, 14.7 mmol) in DMF (10 mL). The resulting mixture was allowed to warm up to room temperature and was then heated at 100° C. for 24 h under nitrogen atmosphere. The progress of the reaction was monitored by TLC. Upon completion of the reaction, the reaction mixture was cooled to 0° C., and then saturated aqueous ammonium chloride solution was added at 0° C. The solvent was removed in vacuo, and the residue was diluted with water and the mixture was extracted with ethyl acetate and the combined organic layers were dried over anhydrous sodium sulfate, filtered and the solvent was removed in vacuo and the residue was purified by Prep-HPLC to afford the title compound (2.5 g, 38.2%) as an off-white solid. ¹H-NMR (400 MHz, DMSO-d₆) δ [ppm]: 2.243 (16.00), 2.518 (2.58), 2.523 (1.68), 6.357 (3.59), 6.835 (2.17), 6.853 (2.38), 6.855 (2.36), 7.107 (0.87), 7.110 (0.93), 7.126 (2.31), 7.129 (2.29), 7.145 (1.59), 7.147 (1.48), 7.202 (1.10), 7.206 (1.20), 7.221 (1.63), 7.226 (1.73), 7.241 (0.72), 7.245 (0.69), 7.348 (1.88), 7.372 (6.46), 7.558 (1.18), 7.561 (1.20), 7.575 (1.51), 7.579 (2.15), 7.582 (1.52), 7.597 (1.54), 7.599 (1.52), 7.754 (1.27), 7.757 (1.40), 7.772 (1.30), 7.775 (1.97), 7.778 (1.58), 7.792 (1.19), 7.796 (1.18), 7.849 (2.33), 7.870 (1.98), 8.291 (2.71), 8.312 (2.43), 12.554 (0.51), LC-MS (Method E, 0-3.00 min 5-95% B): Rₜ=1.63 min; MS (ESIpos): m/z=447 (M+H)⁺. Atropisomeric ratio: atrop 1/atrop 2=49:51 (1% impurities); Rₜ (atrop1)=4.33 min; Rₜ (atrop2)=5.27 min. The atropisomeric ratio was determined using the following chiral HPLC method: Instrument: Agilent HPLC 1260; column: Reprosil Chiral NR 5 μm 100×4.6 mm; eluent A: hexane+0.1 vol-% TFA; eluent B: 2-propanol; isocratic: 65% A+35% B; flow 1.4 mL/min; T: 25° C.; DAD 254 nm. The corresponding atropisomers were separated using preparative chiral HPLC: Instrument: Labomatic HD 5000, Labocord-5000; Gilson GX-241, Labcol Vario 4000; column: Repropsil Chiral NR 5 μm 250×30 mm; Eluent A: hexane+0.1 Vol-% TFA; eluent B: 2-propanol; isocratic: 65% A+35% B; flow 50.0 mL/min; UV 254 nm.

### Example 33

### 3-[4-Chloro-3-(2-methylphenoxy)naphthalen-1-yl]-6-(trifluoromethyl)pyrimidine-2,4(1H,3H)-dione (atrop 1)

optical rotation [α]: 4.2°+/−0.68°

### Example 34

### 3-[4-Chloro-3-(2-methylphenoxy)naphthalen-1-yl]-6-(trifluoromethyl)pyrimidine-2,4(1H,3H)-dione (atrop 2)

optical rotation [α]: −5.1°+/−0.53°

### Example 35

### 4-[2,6-Dioxo-4-(trifluoromethyl)-3,6-dihydropyrimidin-1(2H)-yl]-2-(2-methylphenoxy)naphthalene-1-carbonitrile (rac)

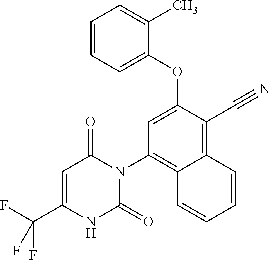 [SMILES: Cc1ccccc1Oc1cc(-n2c(=O)cc(C(F)(F)F)[nH]c2=O)c2ccccc2c1C#N]

According to GP2, to a suspension of sodium hydride (60% in mineral oil) (1.17 g, 29.3 mmol, in 40 mL DMF) was added a solution of commercially available ethyl (2Z)-3-amino-4,4,4-trifluorobut-2-enoate (CAS: 372-29-2, 5.36 g, 29.3 mmol, in 10 mL DMF) at 0° C. and the mixture was stirred for 10 min. Then was slowly added a solution of methyl [4-cyano-3-(2-methylphenoxy)naphthalen-1-yl]carbamate (INT-30, 5.0 g, 14.7 mmol, in 10 mL DMF). The resulting mixture was allowed to warm up to room temperature and was then heated at 100° C. for 24 h under nitrogen atmosphere. Upon completion of the reaction, the reaction mixture was cooled to 0° C., and then saturated aqueous ammonium chloride solution was added at 0° C. The solvent was removed in vacuo and the residue was diluted with water and the mixture was extracted with ethyl acetate and the combined organic layers were dried over anhydrous sodium sulfate, filtered and the solvent was removed in vacuo and the residue was purified by Prep-HPLC (column: Sunfire C₁₈, 5 μm, (19*150 mm); eluent A: 0.1% TFA in Water, eluent B: acetonitrile, flow: 20.0 mL/min; gradient: Time/% B: 0/5, 1/5, 5/20, 5.1/100, 8/100, 8.1/5, 10/5) to afford the title compound (2.50 g, 38%) as an off-white solid. ¹H-NMR (400 MHz, DMSO-d₆) δ [ppm]: 2.18 (s, 3H), 6.33 (s, 1H), 7.16 (d, 1H), 7.24-7.35 (m, 3H), 7.41 (d, 1H), 7.61 (t, 1H), 7.86 (t, 1H), 7.93 (d, 1H), 8.15 (d, 1H), 12.55 (br, 1H); LC-MS (Method C, 0-2.00 min 10-95% B): Rₜ=1.50 min; MS (ESIpos): m/z=438 (M+H)⁺.

The corresponding atropisomers were separated using preparative chiral HPLC: column: CHIRALPAK IA, 2×25 cm, 5 μm; Mobile Phase A: Hex (0.1% TFA)-HPLC, Mobile Phase B: Ethanol-HPLC; Gradient: 10% B to 10% B in 20 min to give 27.2 mg (37% yield) of atrop1 as a white solid and 26.8 mg (36% yield) of atrop2 (second eluting isomer) as a white solid.

### Example 36

### 4-[2,6-Dioxo-4-(trifluoromethyl)-3,6-dihydropyrimidin-1(2H)-yl]-2-(2-methylphenoxy)naphthalene-1-carbonitrile (atrop 1)

tr=10.365. ¹H-NMR (400 MHz, DMSO-d₆): δ [ppm]=2.07 (s, 3H), 6.36 (s, 1H), 7.15 (d, 1H), 7.26 (t, 1H), 7.31-7.35 (m, 2H), 7.41 (d, 1H), 7.60 (t, 1H), 7.86 (t, 1H), 7.95 (d, 1H), 8.15 (d, 1 H), 12.57 (br, 1H); LC-MS (Method C, 0-2.00 min 10-95% B): Rₜ=1.42 min; MS (ESIpos): m/z=438 (M+H)⁺.

### Example 37

### 4-[2,6-Dioxo-4-(trifluoromethyl)-3,6-dihydropyrimidin-1(2H)-yl]-2-(2-methylphenoxy)naphthalene-1-carbonitrile (atrop 2)

Ry=14.427. ¹H-NMR (400 MHz, DMSO-d₆): δ [ppm]=2.18 (s, 3H), 6.36 (s, 1H), 7.15 (d, 1H), 7.25 (t, 1H), 7.31-7.35 (m, 2H), 7.41 (d, 1H), 7.60 (t, 1H), 7.84 (t, 1H), 7.95 (d, 1H), 8.15 (d, 1H), 12.57 (br, 1H); LC-MS (Method C, 0-2.00 min 10-95% B): Rₜ=1.43 min; MS (ESIpos): m/z=438 (M+H)⁺.

### Example 38

### 5-Chloro-2-(2,6-dimethylphenoxy)-4-(2,6-dioxo-4-(trifluoromethyl)-2,3-dihydropyrimidin-1(6H)-yl)benzonitrile (rac)

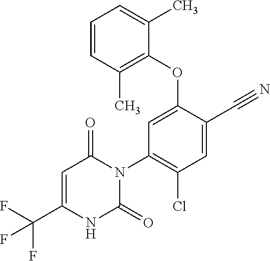 [SMILES: Cc1cccc(C)c1Oc1cc(-n2c(=O)cc(C(F)(F)F)[nH]c2=O)c(Cl)cc1C#N]

According to GP2, to a suspension of sodium hydride (20 mg, 0.48 mmol, 60% in mineral oil, in 3 mL DMF) were added commercially available ethyl 3-amino-4,4,4-trifluorobut-2-enoate (CAS: 372-29-2, 89 mg, 0.48 mmol) and methyl 2-chloro-4-cyano-5-(2,6-dimethylphenoxy)phenylcarbamate (INT-35, 100 mg, 0.24 mmol) at 0° C. The resulting mixture was then heated to 100° C. and was stirred at this temperature overnight under nitrogen atmosphere. After cooling to room temperature, aq. ammonium chloride solution was added at 0° C. to stop the reaction. The solvent was removed in vacuo and the residue was diluted with water. The resulting mixture was extracted with ethyl acetate and the combined organic layers were dried over anhydrous sodium sulfate. The solvent was removed in vacuo and the residue was purified by Prep-HPLC [Column: Xselect Cia 5 μm 19×150 mm, eluent A: Water (0.1% ammonium bicarbonate), eluent B: acetonitrile; gradient: 15% B to 50% B in 8 min] to give the title compound (18.6 mg, 17% yield) as a white solid. ¹H-NMR (400 MHz, DMSO-d₆): δ [ppm]=2.08 (s, 6H), 6.29 (s, 1H), 6.88 (s, 1H), 7.15-7.23 (m, 3H), 8.34 (s, 1H), 12.68 (br, 1H); LC-MS (Method E, 0-3.00 min 5-95% B): Rt=1.58 min; MS (ESIneg): m/z=434 (M−H)⁻.

### Example 39

### 4-(2,6-Dioxo-4-(trifluoromethyl)-2,3-dihydropyrimidin-1(6H)-yl)-5-propoxy-2-(o-tolyloxy)benzonitrile (rac)

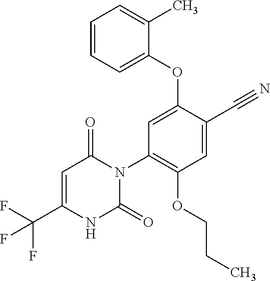 [SMILES: CCCOc1cc(C#N)c(Oc2ccccc2C)cc1-n1c(=O)cc(C(F)(F)F)[nH]c1=O]

According to GP2, to a suspension of sodium hydride (38 mg, 0.9 mmol, 60% in mineral oil, in 5 mL DMF) was added commercially available ethyl 3-amino-4,4,4-trifluorobut-2-enoate (CAS: 372-29-2, 173 mg, 0.9 mmol) at 0° C. After stirred at this temperature for 30 min, methyl 4-cyano-2-propoxy-5-(o-tolyloxy)phenylcarbamate (INT-36, 200 mg, 0.5 mmol) was added to the above mixture and the resulting solution was stirred at 100° C. for overnight under nitrogen atmosphere. After cooling to room temperature, aq. ammonium chloride solution was added at 0° C. and the solvent was removed in vacuo. The residue was purified by Prep-HPLC [eluent A: Water (0.1% ammonium bicarbonate), eluent B: acetonitrile; Gradient: 25% B to 50% B in 8 min] to give the title compound (65.6 mg, 31%) as a white solid. ¹H-NMR (400 MHz, DMSO-d₆): δ [ppm]=0.84 (t, 3H), 1.56-1.64 (m, 2H), 2.19 (s, 3H), 3.99 (t, 2H), 6.27 (s, 1H), 6.94 (s, 1H), 6.98 (d, 1H), 7.17 (t, 1H), 7.26 (t, 1H), 7.35 (d, 1H), 7.72 (s, 1H), 12.54 (br, 1H). LC-MS (Method E, 0-1.50 min 5-95% B): Rₜ=0.98 min; MS (ESIpos): m/z=446 (M+H)⁺.

### Example 40

### 4-{4-[Chloro(difluoro)methyl]-2,6-dioxo-3,6-dihydropyrimidin-1(2H)-yl}-5-fluoro-2-(2-methylphenoxy)benzonitrile

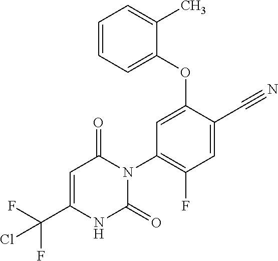 [SMILES: Cc1ccccc1Oc1cc(-n2c(=O)cc(C(F)(F)Cl)[nH]c2=O)c(F)cc1C#N]

According to GP2, to a suspension of sodium hydride (60% in mineral oil, 13.7 mg, 344 μmol, in 40 mL DMF) was added a solution of commercially available ethyl (2Z)-3-amino-4-chloro-4,4-difluorobut-2-enoate (CAS: 136757-13-6, 80.0 mg, 229 μmol, in 0.2 mL DMF) at 0° C. and stirred for 40 min (yellow reaction mixture) until the formation of hydrogen gas could no longer be observed. Then a solution of ethyl [4-cyano-2-fluoro-5-(2-methylphenoxy)phenyl]carbamate (INT-22, (64.0 mg, 321 μmol, in 0.2 mL DMF) was slowly added. The resulting mixture was allowed to warm up to room temperature and was then heated at 90° C. for 19 h under nitrogen atmosphere. The reaction was poured into water (15 mL), the resulting precipitation was filtered off (remaining starting material), washed with water and dried in vacuo. To the aqueous phase (containing the desired product), 2 M HCl (1 mL) was added until pH 3 was reached, and the resulting precipitate was filtered, washed with water and dried in vacuo. The residue was dissolved in DMSO, filtered (Chromafil, 0-45/15 MS, 0.45 μm, Polytetrafluorethylene). and purified using HT-HPLC giving the title compound (28 mg, 28%) as a beige solid. ¹H-NMR (400 MHz, DMSO-d₆) δ [ppm]: 2.153 (16.00), 2.327 (0.69), 2.331 (0.50), 2.518 (2.28), 2.523 (1.67), 2.539 (0.96), 2.669 (0.71), 2.673 (0.50), 6.193 (0.76), 6.945 (0.64), 7.041 (0.98), 7.055 (0.96), 7.073 (0.85), 7.087 (1.89), 7.090 (1.95), 7.108 (2.29), 7.110 (2.26), 7.203 (1.07), 7.206 (0.92), 7.222 (2.29), 7.224 (2.16), 7.240 (1.67), 7.243 (1.45), 7.286 (1.11), 7.290 (1.30), 7.305 (1.61), 7.310 (1.73), 7.325 (0.67), 7.328 (0.63), 7.373 (1.87), 7.375 (1.79), 7.392 (1.51), 8.145 (2.85), 8.168 (2.85); LC-MS (Method IV): Rₜ=1.16 min; MS (ESIpos): m/z=422.

### Example 41

### 4-[2,6-Dioxo-4-(trifluoromethyl)-3,6-dihydropyrimidin-1(2H)-yl]-2-(2-methylphenoxy)benzonitrile

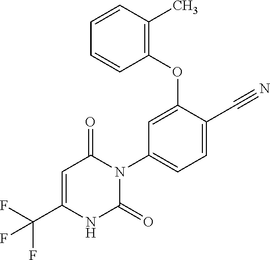 [SMILES: Cc1ccccc1Oc1cc(-n2c(=O)cc(C(F)(F)F)[nH]c2=O)ccc1C#N]

According to GP4, 4-[2,6-dioxo-4-(trifluoromethyl)-3,6-dihydropyrimidin-1(2H)-yl]-2-fluorobenzonitrile (INT-43, 70.0 mg, 234 μmol), commercially available 2-methylphenol (CAS: 95-48-7, 24 μL, 230 μmol) and potassium carbonate (80.8 mg, 585 μmol), were heated in DMSO (2.1 mL) at 110° C. for 20 h. Upon reaction completion, the reaction mixture was filtered and purified using HPLC-HT to give the title compound (50 mg, 52%) as a colourless solid. ¹H-NMR (400 MHz, DMSO-d₆) δ [ppm]: 2.138 (16.00), 2.331 (0.53), 2.518 (2.88), 2.522 (1.82), 2.539 (3.27), 2.673 (0.54), 6.254 (2.24), 6.818 (2.79), 6.822 (2.83), 7.099 (1.86), 7.102 (1.95), 7.119 (2.30), 7.122 (2.28), 7.193 (2.43), 7.197 (2.32), 7.206 (0.96), 7.209 (1.11), 7.214 (2.48), 7.217 (2.42), 7.224 (2.29), 7.228 (2.15), 7.243 (1.60), 7.246 (1.44), 7.287 (1.11), 7.291 (1.31), 7.306 (1.55), 7.310 (1.68), 7.325 (0.62), 7.329 (0.59), 7.370 (1.86), 7.389 (1.45), 7.993 (4.41), 8.013 (4.24); LC-MS (Method IV): Rₜ=1.18 min; MS (ESIpos): m/z=478.

### Example 42

### 4-(2,6-Dioxo-4-phenyl-3,6-dihydropyrimidin-1(2H)-yl)-5-methoxy-2-(2-methylphenoxy)benzonitrile (rac)

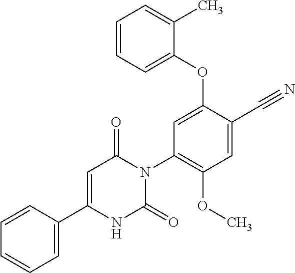 [SMILES: COc1cc(C#N)c(Oc2ccccc2C)cc1-n1c(=O)cc(-c2ccccc2)[nH]c1=O]

A mixture of aforementioned 4-[4-bromo-2,6-dioxo-3,6-dihydropyrimidin-1(2H)-yl]-5-methoxy-2-(2-methylphenoxy)benzonitrile (Example 19, 250 mg, 584 μmol), commercially available phenylboronic acid (CAS: 95-48-7, 71.2 mg, 584 μmol) and commercially available potassium fluoride (CAS: 7789-23-3, 102 mg, 1.75 mmol in dioxane (2.4 mL) were stirred under argon atmosphere at room temperature. Then, commercially available tris(dibenzylideneacetone)dipalladium(0) (CAS: 51364-51-3, 26.7 mg, 29.2 μmol, 0.05 eq.) and commercially available tri-tert-butylphosphoniumtetrafluoroboronate (CAS: 131274-22-1, 16.9 mg, 58.4 μmol) were added to the brown reaction mixture which was then heated to 110° C. for 18 h. Upon reaction completion the reaction mixture was concentrated and then purified using HPLC-HT giving the title compound as a yellow glittery solid (30 mg, 11%). ¹H-NMR (400 MHz, DMSO-d₆) δ [ppm]: 2.074 (2.36), 2.206 (12.43), 2.518 (1.71), 2.522 (1.05), 2.539 (0.54), 3.813 (16.00), 5.988 (3.71), 6.946 (1.71), 6.967 (2.18), 6.972 (7.95), 7.138 (0.75), 7.140 (0.76), 7.156 (1.86), 7.158 (1.80), 7.174 (1.24), 7.177 (1.15), 7.240 (0.88), 7.243 (0.97), 7.263 (1.39), 7.279 (0.57), 7.282 (0.56), 7.341 (1.51), 7.360 (1.25), 7.487 (0.73), 7.491 (1.15), 7.495 (0.53), 7.509 (3.32), 7.513 (1.58), 7.527 (3.16), 7.538 (1.08), 7.542 (1.94), 7.546 (1.15), 7.554 (0.60), 7.560 (1.79), 7.578 (0.45), 7.733 (3.04), 7.740 (7.47), 7.750 (1.64), 7.754 (3.06), 11.537 (1.50); LC-MS (Method I): Rₜ=1.14 min; MS (ESIpos): m/z=426 [M+H]⁺. Atropisomeric ratio: atrop 1/atrop 2=50:47 (3% impurities); Rₜ (atrop1)=2.73 min; Rₜ (atrop2)=3.38 min. The atropisomeric ratio was determined using the following chiral HPLC method: Instrument: Agilent HPLC 1260; column: cellulose SC 3 μm 100×4.6 mm; eluent A: hexane+0.1 vol-% TFA; eluent B: 2-propanol; gradient: 20-50% B in 7 min; flow 1.4 mL/min; T: 25° C.; DAD 254 nm. The corresponding atropisomers were separated using preparative chiral HPLC: Instrument: Labomatic HD5000, Labocord-5000; Gilson GX-241, Labcol Vario 4000; column: cellulose SC 5 μm 250×30 mm; eluent A: hexane+0.1 Vol-% TFA; eluent B: 2-propanol; gradient: 20-50% B in 7 min; flow 40.0 mlLmin; UV 254 nm.

### Example 43

### 4-(2,6-Dioxo-4-phenyl-3,6-dihydropyrimidin-1(2H)-yl)-5-methoxy-2-(2-methylphenoxy)benzonitrile (atrop 1)

white solid, 8 mg.

### Example 44

### 4-(2,6-Dioxo-4-phenyl-3,6-dihydropyrimidin-1(2H)-yl)-5-methoxy-2-(2-methylphenoxy)benzonitrile (atrop 2)

white solid, 7 mg.

### Example 45

### 4-[2,6-Dioxo-4-(trifluoromethyl)-3,6-dihydropyrimidin-1(2H)-yl]-5-fluoro-2-(2-methylphenoxy)benzonitrile

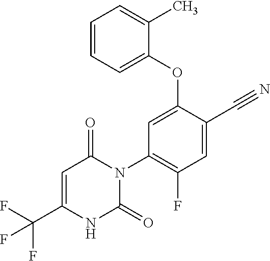 [SMILES: Cc1ccccc1Oc1cc(-n2c(=O)cc(C(F)(F)F)[nH]c2=O)c(F)cc1C#N]

According to GP4, literature known 4-[2,6-dioxo-4-(trifluoromethyl)-3,6-dihydropyrimidin-1(2H)-yl]-2,5-difluorobenzonitrile (DE 10307142 A1 20040902, CAS: 162926-25-2, 500 mg, 1.58 mmol), commercially available 2-methylphenol (CAS: 95-48-7, 160 μL, 1.6 mmol) and potassium carbonate (545 mg, 3.94 mmol) were heated in DMSO (5.5 mL) in two separate vials at 110° C. for 20 h. Upon reaction completion, the reaction was filtered and purified using HPLC-HT to give the title compound (409 mg, 61%) as a colourless solid. ¹H-NMR (400 MHz, DMSO-d₆) δ [ppm]: 2.148 (16.00), 2.518 (2.17), 2.523 (1.47), 2.539 (2.90), 6.349 (3.51), 7.048 (2.89), 7.063 (2.89), 7.097 (1.88), 7.100 (2.05), 7.117 (2.30), 7.119 (2.38), 7.209 (0.83), 7.211 (0.88), 7.227 (2.29), 7.230 (2.22), 7.245 (1.69), 7.249 (1.55), 7.291 (1.09), 7.294 (1.29), 7.309 (1.58), 7.314 (1.77), 7.329 (0.64), 7.332 (0.61), 7.376 (1.82), 7.378 (1.77), 7.395 (1.48), 7.397 (1.36), 8.156 (3.77), 8.179 (3.77); LC-MS (Method E, 0-3.00 min 5-95% B): Rₜ=1.41 min; MS (ESIneg): m/z=404 (M−H)⁻.

### Example 46

### 2-(2-Chlorophenoxy)-4-[2,6-dioxo-4-(trifluoromethyl)-3,6-dihydropyrimidin-1 (2H)-yl]benzonitrile

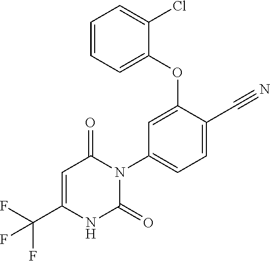 [SMILES: N#Cc1ccc(-n2c(=O)cc(C(F)(F)F)[nH]c2=O)cc1Oc1ccccc1Cl]

According to GP4, 4-[2,6-dioxo-4-(trifluoromethyl)-3,6-dihydropyrimidin-1(2H)-yl]-2-fluorobenzonitrile (INT-43, 100 mg, 334 μmol), commercially available 2-chlorophenol (CAS: 95-57-8, 43.0 mg, 334 μmol) and potassium carbonate (115 mg, 836 μmol) were heated in DMSO (2 mL) at 110° C. for 21 h. Upon reaction completion, the reaction mixture was filtered and purified using HPLC-HT to give the title compound (29 mg, 20%) as a light brown solid. ¹H-NMR (400 MHz, DMSO-d₆) δ [ppm]: 1.315 (0.40), 2.074 (1.89), 2.331 (0.43), 2.518 (2.23), 2.522 (1.49), 2.539 (0.98), 2.673 (0.43), 6.273 (14.13), 6.924 (12.30), 6.927 (12.45), 6.946 (0.54), 7.073 (0.54), 7.201 (0.52), 7.253 (8.22), 7.258 (7.50), 7.274 (8.18), 7.278 (8.32), 7.339 (3.02), 7.343 (3.91), 7.355 (6.03), 7.359 (14.48), 7.379 (16.00), 7.452 (5.57), 7.456 (5.84), 7.470 (5.43), 7.472 (5.16), 7.474 (5.41), 7.491 (2.76), 7.495 (2.76), 7.657 (8.51), 7.660 (6.18), 7.665 (2.18), 7.678 (8.80), 7.682 (4.36), 8.026 (13.81), 8.046 (13.10), 12.471 (1.47); LC-MS (Method I): Rₜ=1.08 min; MS (ESIneg): m/z=406 [M−H]⁻.

### Example 47

### 5-[2,6-Dioxo-4-(trifluoromethyl)-3,6-dihydropyrimidin-1(2H)-yl]-3-(2-methylphenoxy)pyridine-2-carbonitrile

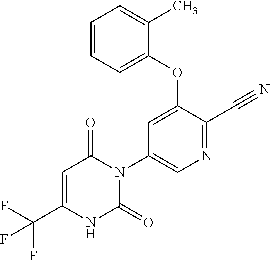 [SMILES: Cc1ccccc1Oc1cc(-n2c(=O)cc(C(F)(F)F)[nH]c2=O)cnc1C#N]

According to GP4, 5-[2,6-dioxo-4-(trifluoromethyl)-3,6-dihydropyrimidin-1(2H)-yl]-3-fluoropyridine-2-carbonitrile (INT-44, 84.0 mg, 280 μmol), commercially available 2-methylphenol (CAS: 95-48-7, 29 μL, 280 μmol) and potassium carbonate (96.7 mg, 700 μmol) were heated in DMSO (2 mL) at 110° C. for 21 h. Upon reaction completion, the reaction mixture was filtered and purified using HPLC-HT to give the title compound (40 mg, 33%) as an ochre solid. ¹H-NMR (400 MHz, DMSO-d₆) δ [ppm]: 2.150 (16.00), 2.518 (0.65), 2.523 (0.44), 6.348 (5.52), 7.205 (1.73), 7.208 (1.82), 7.225 (2.38), 7.228 (2.52), 7.255 (0.76), 7.259 (0.81), 7.274 (2.26), 7.277 (2.06), 7.292 (1.84), 7.295 (1.47), 7.322 (1.21), 7.325 (1.45), 7.341 (1.53), 7.345 (1.66), 7.360 (0.59), 7.363 (0.55), 7.407 (1.85), 7.410 (1.78), 7.426 (1.47), 7.446 (5.86), 7.450 (5.95), 8.438 (5.66), 8.443 (5.81); LC-MS (Method I): Rₜ=1.09 min; MS (ESIpos): m/z=389 [M+H]⁺.

### Example 48

### 2-(2,6-Dimethylphenoxy)-4-[2,6-dioxo-4-(trifluoromethyl)-3,6-dihydropyrimidin-1 (2H)-yl]benzonitrile

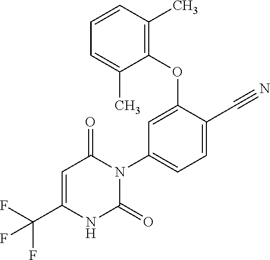 [SMILES: Cc1cccc(C)c1Oc1cc(-n2c(=O)cc(C(F)(F)F)[nH]c2=O)ccc1C#N]

According to GP4, 4-[2,6-dioxo-4-(trifluoromethyl)-3,6-dihydropyrimidin-1(2H)-yl]-2-fluorobenzonitrile (INT-43, 100 mg, 334 μmol), commercially available 2,6-dimethylphenol (CAS: 576-26-1, 40.8 mg, 334 μmol) and potassium carbonate (115 mg, 836 μmol) were heated in DMSO (2 mL) at 110° C. for 21 h. Upon reaction completion, the reaction mixture was filtered and purified using HPLC-HT to give the title compound (11 mg, 8%) as reddish brown solid. ¹H-NMR (400 MHz, DMSO-d₆) δ [ppm]: 2.075 (0.89), 2.086 (16.00), 2.523 (0.58), 2.540 (0.49), 6.248 (2.26), 6.630 (1.88), 6.635 (1.87), 7.135 (0.44), 7.149 (0.77), 7.155 (1.86), 7.159 (1.53), 7.171 (1.87), 7.175 (1.56), 7.180 (1.44), 7.195 (3.10), 7.211 (1.00), 7.992 (2.10), 8.012 (2.01); LC-MS (Method I): Rₜ=1.15 min; MS (ESIneg): m/z=400 [M−H]⁻.

### Example 49

### 4-(2,6-Dioxo-4-phenyl-3,6-dihydropyrimidin-1(2H)-yl)-5-fluoro-2-(2-methylphenoxy)benzonitrile

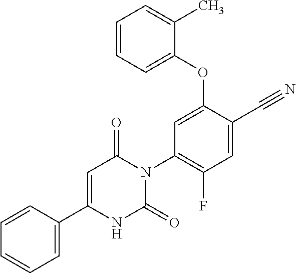 [SMILES: Cc1ccccc1Oc1cc(-n2c(=O)cc(-c3ccccc3)[nH]c2=O)c(F)cc1C#N]

A mixture of aforementioned 4-[2,6-Dioxo-4-(trifluoromethyl)-3,6-dihydropyrimidin-1(2H)-yl]-5-methoxy-2-(2-methylphenoxy)benzonitrile (Example 3, 50.0 mg, 120 μmol), commercially available phenylboronic acid (CAS: 98-80-6, 24.5 mg, 120 μmol) and commercially available potassium fluoride (CAS: 7789-23-3, 20.9 mg, 360 μmol) in dioxane (500 μL) was stirred under argon atmosphere at room temperature. Then, commercially available tris(dibenzylideneacetone)dipalladium(0) (CAS: 51364-51-3, 5.50 mg, 6.01 μmol, 0.05 eq.) and commercially available tri-tert-butylphosphoniumtetrafluoroboronate (CAS: 131274-22-1, 3.49 mg, 12.0 μmol μmol) were added to the brown reaction mixture which was heated to 110° C. for 18 h. Upon reaction completion the reaction mixture was purified using HPLC-HT to give the title compound as two fractions with different purity: one as a light yellow solid (2 mg, 3%, ca. 80% pure) and the second one as a white solid (2 mg, 4%). ¹H-NMR (400 MHz, CHLOROFORM-d) δ [ppm]:—0.014 (0.61),—0.008 (2.91), 0.008 (3.55), 0.013 (1.06), 0.070 (12.88), 0.077 (2.20), 0.079 (2.07), 0.095 (0.65), 0.174 (0.87), 0.881 (0.49), 1.253 (1.04), 1.308 (0.54), 1.465 (0.58), 1.497 (0.57), 1.582 (16.00), 2.012 (1.24), 2.212 (0.53), 2.220 (0.55), 2.236 (14.80), 2.622 (1.14), 6.031 (3.15), 6.035 (3.22), 6.657 (3.43), 6.671 (3.36), 6.999 (0.46), 7.027 (1.59), 7.030 (1.62), 7.047 (1.96), 7.050 (1.96), 7.142 (0.73), 7.158 (1.89), 7.161 (1.86), 7.176 (1.60), 7.179 (1.41), 7.204 (1.07), 7.207 (1.30), 7.222 (1.38), 7.227 (1.54), 7.247 (0.91), 7.289 (1.21), 7.495 (1.12), 7.514 (2.97), 7.522 (0.79), 7.526 (1.08), 7.535 (5.05), 7.542 (3.92), 7.547 (6.13), 7.556 (3.88), 7.560 (1.51), 7.564 (2.16), 7.568 (1.24), 7.574 (1.18), 7.578 (1.21), 7.590 (0.76), 7.596 (2.02), 7.603 (0.45), 7.609 (0.43), 7.613 (0.69), 8.498 (0.97); LC-MS (Method I): Rₜ=1.17 min; MS (ESIpos): m/z=413 [M−H]⁺.

### Example 50

### 3-[4-Chloro-5-(2-chlorophenoxy)-2-methoxyphenyl]-6-(trifluoromethyl)pyrimidine-2,4(1H,3H)-dione (rac)

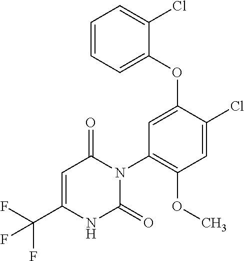 [SMILES: COc1cc(Cl)c(Oc2ccccc2Cl)cc1-n1c(=O)cc(C(F)(F)F)[nH]c1=O]

According to GP3, INT-45 (160 mg, 400 μmol), commercially available 2-chlorophenol (CAS: 95-57-8, 73.1 mg, 569 μmol), N,N-dimethylglycine (10.9 mg, 106 μmol), copper (i) iodide (10.3 mg, 54.1 μmol) and cesium carbonate (313 mg, 961 μmol) were heated in DMF (7.9 mL) for 18 h at 140° C. Upon reaction completion, work-up and two purifications the title compound was obtained as a brown solid with low purity (3 mg, 1%). ¹H-NMR (400 MHz, METHANOL-d₄) δ [ppm]: 1.221 (0.48), 2.209 (0.69), 2.659 (5.58), 3.011 (0.48), 3.847 (16.00), 3.991 (0.93), 6.215 (2.97), 6.717 (0.40), 6.736 (0.76), 6.755 (0.53), 6.827 (1.40), 6.831 (1.43), 6.848 (1.53), 6.851 (1.59), 7.046 (0.96), 7.052 (5.72), 7.064 (1.24), 7.068 (1.26), 7.084 (1.04), 7.088 (1.01), 7.140 (0.43), 7.144 (0.44), 7.160 (0.41), 7.195 (1.03), 7.198 (1.15), 7.213 (1.01), 7.215 (1.13), 7.218 (1.00), 7.219 (1.02), 7.234 (0.75), 7.238 (0.78), 7.356 (4.77), 7.452 (1.56), 7.456 (1.60), 7.472 (1.49), 7.476 (1.43); LC-MS (Method I): Rₜ=1.20 min; MS (ESIpos): m/z=446 [M−H]⁺.

### Example 51

### 4-[2,6-Dioxo-4-(pentafluoroethyl)-3,6-dihydropyrimidin-1(2H)-yl]-5-methoxy-2-(2-methylphenoxy)benzonitrile (rac)

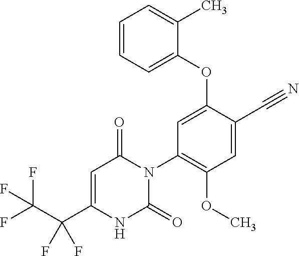 [SMILES: COc1cc(C#N)c(Oc2ccccc2C)cc1-n1c(=O)cc(C(F)(F)C(F)(F)F)[nH]c1=O]

According to GP3, 2-bromo-4-[2,6-dioxo-4-(pentafluoroethyl)-3,6-dihydropyrimidin-1(2H)-yl]-5-methoxybenzonitrile (INT-46, 90.0 mg, 204 μmol), commercially available 2-methylphenol (CAS: 95-48-7, 31.4 mg, 290 μmol), N,N-dimethylglycine (5.59 mg, 54.2 μmol), copper(I) iodide (5.26 mg, 27.6 μmol) and cesium carbonate (160 mg, 491 μmol) were heated in DMF (2 mL) for 18 h at 140° C. Upon reaction completion, work-up and two purifications, the title compound was obtained as a brown solid with low purity (10 mg, 9%). ¹H-NMR (400 MHz, DMSO-d₆) δ [ppm]: 2.117 (0.50), 2.160 (1.45), 2.167 (2.14), 2.181 (13.71), 2.323 (1.57), 2.327 (2.14), 2.332 (1.60), 2.522 (5.84), 2.665 (1.53), 2.669 (2.21), 2.673 (1.57), 3.775 (0.69), 3.797 (16.00), 3.847 (0.42), 6.261 (0.61), 6.967 (1.79), 6.987 (2.44), 7.150 (0.84), 7.166 (1.95), 7.184 (1.37), 7.249 (1.03), 7.268 (1.45), 7.287 (0.65), 7.343 (1.60), 7.362 (1.37), 7.739 (4.43), 8.259 (0.50), 8.428 (0.61), 9.526 (0.50); LC-MS (Method I): Rₜ=1.20 min; MS (ESIpos): m/z=468 [M+H]⁺.

### Example 52

### 4-[2,6-dioxo-4-(pentafluoroethyl)-3,6-dihydropyrimidin-1(2H)-yl]-5-fluoro-2-(2-methylphenoxy)benzonitrile

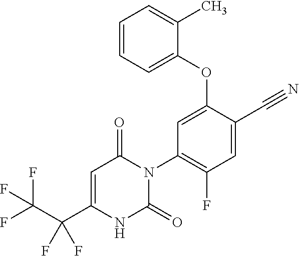 [SMILES: Cc1ccccc1Oc1cc(-n2c(=O)cc(C(F)(F)C(F)(F)F)[nH]c2=O)c(F)cc1C#N]

According to GP2, to a suspension of sodium hydride (60% in mineral oil, 16.3 mg, 408 μmol) in 710 μL DMF was added a solution of commercially available ethyl (2Z)-3-amino-4,4,5,5,5-pentafluoropent-2-enoate (CAS: 72850-56-7, 90.0 mg, 272 μmol) at 0° C., and the mixture was stirred for 40 min (yellow reaction mixture) the formation of hydrogen gas could no longer be observed. Then a solution of methyl [4-cyano-2-fluoro-5-(2-methylphenoxy)phenyl]carbamate (INT-29, 88.8 mg, 381 μmol, in 0.3 mL DMF) was slowly added. The resulting mixture was allowed to warm up to room temperature and was then heated at 90° C. for 19 h under nitrogen atmosphere. Then, the reaction mixture was poured into water, the resulting precipitate was filtered off (remaining starting material), washed with water and dried in vacuo. To the aqueous phase (containing the desired product), 2 M aq. HCl (1 mL) was added until pH 3 was reached, and the resulting precipitate was filtered, washed with water and dried in vacuo. The residue was dissolved in DMSO, filtered and purified using HT-HPLC giving the title compound as a colourless solid (46.8 mg, 34%). Atropisomeric ratio: atrop 1/atrop 2=50:50; Rₜ (atrop1)=1.38 min; Rₜ (atrop2)=2.28 min. The atropisomeric ratio was determined using the following chiral HPLC method: Instrument: Agilent HPLC 1260; column: Amylose SB 3 μm 100×4.6 mm; eluent A: hexane+0.1 vol-% TFA (99%); eluent B: 2-propanol; gradient: 20-50% B in 7 min; flow 1.4 mL/min; T: 25° C.; DAD 254 nm. ¹H-NMR (400 MHz, DMSO-d₆) δ [ppm]: 2.157 (16.00), 2.518 (2.45), 2.523 (1.79), 2.539 (2.21), 6.264 (0.72), 6.944 (0.91), 7.024 (1.01), 7.038 (1.01), 7.072 (1.00), 7.092 (1.83), 7.095 (1.92), 7.112 (2.25), 7.114 (2.26), 7.200 (0.97), 7.203 (0.93), 7.206 (0.88), 7.222 (2.23), 7.225 (2.16), 7.240 (1.66), 7.244 (1.47), 7.287 (1.09), 7.291 (1.26), 7.306 (1.57), 7.311 (1.71), 7.326 (0.66), 7.329 (0.64), 7.374 (1.83), 7.376 (1.76), 7.393 (1.51), 8.149 (2.85), 8.171 (2.85).

### Example 53

### 5-Chloro-4-{4-[difluoro(phenyl)methyl]-2,6-dioxo-3,6-dihydropyrimidin-1(2H)-yl}-2-(2-methylphenoxy)benzonitrile (rac)

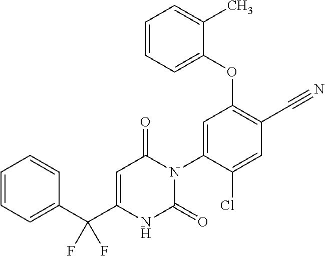 [SMILES: Cc1ccccc1Oc1cc(-n2c(=O)cc(C(F)(F)c3ccccc3)[nH]c2=O)c(Cl)cc1C#N]

According to GP2, to a suspension of sodium hydride (18 mg, 0.4 mmol, 60% in mineral oil) in DMF (3 mL) were added ethyl 3-amino-4,4-difluoro-4-phenylbut-2-enoate (INT-3, 107 mg, 0.44 mmol) and methyl [2-chloro-4-cyano-5-(2-methylphenoxy)phenyl]carbamate (INT-32, 100 mg, 0.22 mmol, 70% purity) at 0° C. The resulting mixture was stirred at 130° C. for 24 h under nitrogen atmosphere. After cooling to room temperature, aq. ammonium chloride solution was added at 0° C. and the solvent was removed in vacuo. The residue was re-dissolved with water and the resulting mixture was extracted with ethyl acetate. The combined organic layers were dried over anhydrous sodium sulfate and concentrated in vacuo. The residue was purified by Prep-HPLC [eluent A: water (10 mmol/L ammonium bicarbonate), eluent B: acetonitrile; Gradient: 30% B to 70% B in 8 min] to give the title compound (17.5 mg, 16%) as a white solid.

¹H-NMR (400 MHz, DMSO-d₆): δ [ppm]=2.13 (s, 3H), 6.01 (s, 1H), 7.09 (s, 1H), 7.11-7.13 (m, 2H), 7.23 (t, 1H), 7.31 (t, 1H), 7.38 (t, 1H), 7.54-7.59 (m, 3H), 7.69 (d, 2H), 8.32 (s, 1H), 12.15 (s, 1H); LC-MS (Method E, 0-3.00 min 5-95% B): Rₜ=1.62 min; MS (ESIneg): m/z=478 (M−H)⁻.

### Example 54

### 4-[2,6-Dioxo-4-(trifluoromethyl)-3,6-dihydropyrimidin-1(2H)-yl]-5-hydroxy-2-(2-methylphenoxy)benzonitrile (rac)

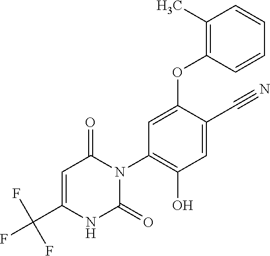 [SMILES: Cc1ccccc1Oc1cc(-n2c(=O)cc(C(F)(F)F)[nH]c2=O)c(O)cc1C#N]

To a solution of aforementioned 5-bromo-4-[2,6-dioxo-4-(trifluoromethyl)-3,6-dihydropyrimidin-1(2H)-yl]-2-(2-methylphenoxy)benzonitrile (18 1.0 g, 1.9 mmol) in dry 1,4-dioxane (30 mL) were added bis(pinacolato)diboron (1.0 g, 3.9 mmol), potassium acetate (570 mg, 5.8 mmol) and [1,1′-Bis(diphenylphosphino)ferrocene]dichloropalladium(ii) (141 mg, 0.2 mmol). The resulting mixture was stirred at 90° C. for overnight under nitrogen atmosphere. After cooling to room temperature, hydrogen peroxide (0.6 mL, 5.8 mmol, 30% aqueous solution) was added and the resulting mixture was stirred at room temperature for another 1 h. Upon completion of the reaction, the solvent was removed in vacuo and the residue was diluted with water. The resulting mixture was extracted with ethyl acetate and the combined organic layers were dried over anhydrous sodium sulfate. The solvent was removed in vacuo and the residue was purified by Prep-HPLC [eluent A: water, eluent B: acetonitrile; gradient 30% B to 60% B in 15 min] to give the title compound (250 mg, 26% yield) as a white solid. ¹H-NMR (400 MHz, DMSO-d₆): δ [ppm]=2.08 (5, 3H), 6.29 (s, 1H), 6.90-6.93 (m, 2H), 7.13 (t, 1H), 7.24 (t, 1H), 7.32-7.35 (m, 2H), 10.28 (s, 1H), 12.67 (br, 1H); LC-MS (Method B, 0-3.00 min 5-95% B): Rₜ=1.34 min; MS (ESIpos): m/z=404 (M+H)⁺.

### Example 55

### 4-[2,6-Dioxo-4-(trifluoromethyl)-3,6-dihydropyrimidin-1(2H)-yl]-2-(2-methylphenoxy)-5-(trifluoromethoxy)benzonitrile (rac)

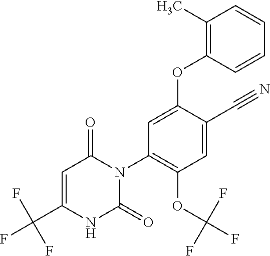 [SMILES: Cc1ccccc1Oc1cc(-n2c(=O)cc(C(F)(F)F)[nH]c2=O)c(OC(F)(F)F)cc1C#N]

To a solution of the aforementioned 4-[2,6-dioxo-4-(trifluoromethyl)-3,6-dihydropyrimidin-1(2H)-yl]-5-hydroxy-2-(2-methylphenoxy)benzonitrile (Example 54, 200 mg, 0.4 mmol, 82% purity) in (trifluoromethyl)benzene/toluene (12 mL, v:v=2:1) were added silver (I) triflate (522 mg, 2.0 mmol), Selectfluor® (288 mg, 0.8 mmol), NFSI (256 mg, 0.8 mmol), cesium fluoride (371 mg, 2.4 mmol), 2-fluoropyridine (189 mg, 2.0 mmol), and trimethyl(trifluoromethyl)silane (289 mg, 2.0 mmol). The resulting mixture was stirred at room temperature overnight under nitrogen atmosphere. Upon completion of the reaction, water was added and the resulting mixture was extracted with ethyl acetate. The combined organic layers were dried over anhydrous sodium sulfate and concentrated in vacuo. The residue was purified by Prep-HPLC [column: Xselect C₁₈ 5 μm 19×150 mm, eluent A: Water (0.1% ammonium bicarbonate), eluent B: acetonitrile; Gradient: 25% B to 55% B in 8 min] to give the title compound (5.2 mg, 3%) as a white solid. ¹H-NMR (400 MHz, DMSO-d₆): δ [ppm]=2.15 (s, 3H), 6.21 (br, 1H), 7.01 (5, 1H), 7.17 (d, 1H), 7.25 (t, 1H), 7.35 (t, 1H), 7.40 (d, 1H), 8.29 (s, 1H), 12.68 (br, 1H); LC-MS (Method E, 0-3.00 min 5-95% B): Rₜ=1.45 min; MS (ESIneg): m/z=470 (M−H)⁻.

### Example 56

### 4-{4-[Difluoro(phenyl)methyl]-2,6-dioxo-3,6-dihydropyrimidin-1(2H)-yl}-5-fluoro-2-(2-methylphenoxy)benzonitrile

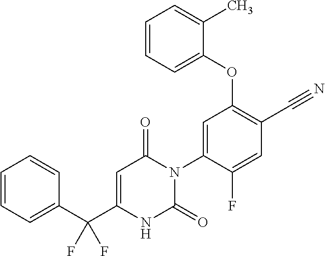 [SMILES: Cc1ccccc1Oc1cc(-n2c(=O)cc(C(F)(F)c3ccccc3)[nH]c2=O)c(F)cc1C#N]

According to GP2, to a solution of sodium hydride (60% in mineral oil, 27 mg, 0.7 mmol) in DMF (5 mL) were added ethyl 3-amino-4,4-difluoro-4-phenylbut-2-enoate (INT-3, 180 mg, 0.7 mmol) and methyl [4-cyano-2-fluoro-5-(2-methylphenoxy)phenyl]carbamate (INT-29, 150 mg, 0.5 mmol) at 0° C. The resulting mixture was stirred at 130° C. overnight under nitrogen atmosphere. Upon completion of the reaction, aq. ammonium chloride solution was added at 0° C. and the resulting solution was extracted with ethyl acetate. The combined organic layer was dried over anhydrous sodium sulfate and concentrated in vacuo. The residue was purified by Prep-HPLC [eluent A: Water (0.1% ammonium bicarbonate), eluent B: acetonitrile; gradient: 20% B to 55% B in 7 min] to give the title compound (47.9 mg, 15%) as a white solid. ¹H-NMR (400 MHz, DMSO-d₆): δ [ppm]=2.15 (s, 3H), 6.00 (s, 1H), 7.05-7.09 (m, 2H), 7.21 (t, 1H), 7.30 (t, 1H), 7.37 (d, 1H), 7.53-7.59 (m, 3H), 7.69 (d, 2H), 8.13 (d, 1H), 12.18 (br, 1H); LC-MS (Method C, 0-2.00 min 10-95% B): Rₜ=1.56 min; MS (ESIneg): m/z=462 (M−H)⁻.

### Example 57

### 4-{4-[Difluoro(phenyl)methyl]-2,6-dioxo-3,6-dihydropyrimidin-1(2H)-yl}-2-(2,6-dimethylphenoxy)-5-fluorobenzonitrile

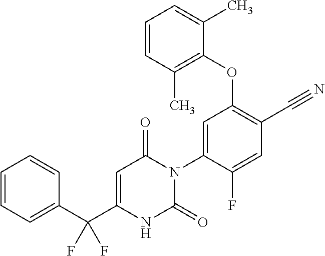 [SMILES: Cc1cccc(C)c1Oc1cc(-n2c(=O)cc(C(F)(F)c3ccccc3)[nH]c2=O)c(F)cc1C#N]

According to GP2, to a suspension of sodium hydride (20 mg, 0.51 mmol, 60% in mineral oil) in DMF (3 mL) were added ethyl 3-amino-4,4-difluoro-4-phenylbut-2-enoate (INT-3, 123 mg, 0.51 mmol) and methyl 4-cyano-5-(2,6-dimethylphenoxy)-2-fluorophenylcarbamate (INT-37, 100 mg, 0.26 mmol, 80% purity) at 0° C. The resulting mixture was stirred at 130° C. for 24 h under nitrogen atmosphere. After cooling to room temperature, aq. ammonium chloride solution was added at 0° C. and the solvent was removed in vacuo. The residue was re-dissolved with water and the resulting mixture was extracted with ethyl acetate. The combined organic layers were dried over anhydrous sodium sulfate and concentrated in vacuo. The residue was purified by Prep-HPLC [column: Gemini C₁₈ 30×100 mm; eluent A: water (0.1% ammoniac), eluent B: acetonitrile; gradient: 20% B to 62% B in 8 min] to give the title compound (30.6 mg, 24%) as a white solid. ¹H-NMR (400 MHz, DMSO-d₆): δ [ppm]=2.08 (s, 6H), 6.00 (s, 1H), 6.83 (s, 1H), 7.13-7.21 (m, 3H), 7.53-7.61 (m, 3H), 7.69 (d, 2H), 8.15 (d, 1H), 12.14 (s, 1H); LC-MS (Method E, 0-3.00 min 5-95% B): Rₜ=1.67 min; MS (ESIneg): m/z=476 (M−H)⁻.

### Example 58

### 2-(2-Acetyl-4,6-dimethylphenoxy)-4-[2,6-dioxo-4-(trifluoromethyl)-3,6-dihydropyrimidin-1(2H)-yl]-5-fluorobenzonitrile

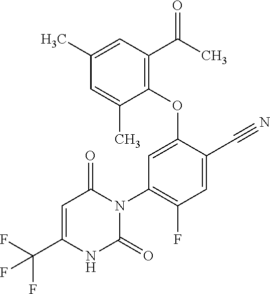 [SMILES: CC(=O)c1cc(C)cc(C)c1Oc1cc(-n2c(=O)cc(C(F)(F)F)[nH]c2=O)c(F)cc1C#N]

According to GP4, literature known 4-[2,6-dioxo-4-(trifluoromethyl)-3,6-dihydropyrimidin-1(2H)-yl]-2,5-difluorobenzonitrile (DE 10307142 A1 20040902, CAS: 162926-25-2, 100 mg, 315 μmol), commercially available 1-(2-hydroxy-3,5-dimethylphenyl)ethan-1-one (CAS: 1198-66-9, 51.8 mg, 315 μmol), and potassium carbonate (109 mg, 788 μmol) were heated in DMSO (2 mL) at 110° C. for 21 h. Upon reaction completion, the reaction mixture was filtered and purified using HPLC-HT to give the title compound (8 mg, 5%) as an ochre solid. ¹H-NMR (400 MHz, DMSO-d₆) δ [ppm]: 2.074 (6.81), 2.140 (0.62), 2.349 (6.79), 2.420 (0.62), 2.438 (16.00), 2.518 (2.12), 2.523 (1.68), 2.540 (0.76), 6.315 (0.57), 6.840 (0.55), 6.853 (0.54), 7.423 (1.20), 7.425 (1.16), 7.429 (1.32), 7.561 (1.31), 7.567 (1.24), 8.130 (1.49), 8.152 (1.50); LC-MS (Method IV): Rₜ=1.14 min; MS (ESIpos): m/z=462.

### Example 59

### 4-[2,6-Dioxo-4-(trifluoromethyl)-3,6-dihydropyrimidin-1(2H)-yl]-2-(2-ethyl-6-methylphenoxy)-5-fluorobenzonitrile

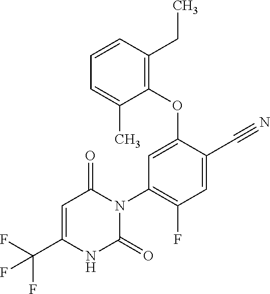 [SMILES: CCc1cccc(C)c1Oc1cc(-n2c(=O)cc(C(F)(F)F)[nH]c2=O)c(F)cc1C#N]

According to GP4, literature known 4-[2,6-dioxo-4-(trifluoromethyl)-3,6-dihydropyrimidin-1(2H)-yl]-2,5-difluorobenzonitrile (DE 10307142 A1 20040902, CAS: 162926-25-2, 100 mg, 315 μmol), commercially available 2-ethyl-6-methylphenol (CAS: 1687-64-5, 42.9 mg, 315 μmol), and potassium carbonate (109 mg, 788 μmol) were heated in DMSO (2.1 mL) at 110° C. for 21 h. Upon reaction completion, the reaction mixture was filtered and purified using HPLC-HT to give the title compound (23 mg, 16%) as an ochre solid. ¹H-NMR (400 MHz, DMSO-d₆) δ [ppm]: 1.076 (5.43), 1.095 (11.79), 1.114 (5.67), 2.071 (15.74), 2.442 (2.19), 2.459 (2.19), 2.518 (1.85), 2.523 (1.32), 2.539 (1.11), 6.333 (8.25), 6.836 (5.40), 6.851 (5.35), 7.182 (0.72), 7.201 (3.39), 7.209 (2.43), 7.216 (16.00), 7.227 (6.27), 7.237 (2.12), 7.241 (1.16), 7.251 (1.02), 8.150 (6.45), 8.172 (6.25); LC-MS (Method IV): Rₜ=1.19 min; MS (ESIpos): m/z=434.

### Example 60

### 4-[2,6-Dioxo-4-(trifluoromethyl)-3,6-dihydropyrimidin-1(2H)-yl]-5-fluoro-2-(2,3,6-trimethylphenoxy)benzonitrile

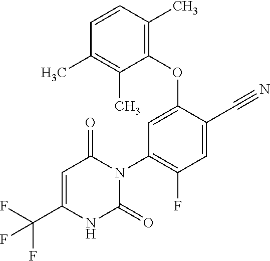 [SMILES: Cc1ccc(C)c(Oc2cc(-n3c(=O)cc(C(F)(F)F)[nH]c3=O)c(F)cc2C#N)c1C]

According to GP4, literature known 4-[2,6-dioxo-4-(trifluoromethyl)-3,6-dihydropyrimidin-1(2H)-yl]-2,5-difluorobenzonitrile (DE 10307142 A1 20040902, CAS: 162926-25-2, 100 mg, 315 μmol), commercially available 2,3,6-trimethylphenol (CAS: 2416-94-6, 42.9 mg, 315 μmol), and potassium carbonate (109 mg, 788 μmol) were heated in DMSO (2 mL) at 110° C. for 21 h. Upon reaction completion, the reaction mixture was filtered and purified using HPLC-HT to give the title compound (28 mg, 17%) as an ochre solid. ¹H-NMR (400 MHz, DMSO-d₆) δ [ppm]: 1.988 (2.05), 2.002 (16.00), 2.035 (13.54), 2.240 (15.31), 2.518 (1.36), 2.523 (0.93), 2.539 (1.79), 3.351 (0.70), 6.339 (5.23), 6.364 (0.72), 6.785 (0.49), 6.796 (3.17), 6.811 (3.13), 6.960 (0.41), 6.986 (0.44), 7.001 (0.44), 7.056 (1.28), 7.076 (5.05), 7.088 (4.61), 7.107 (1.16), 8.134 (0.56), 8.147 (4.29), 8.157 (0.68), 8.169 (4.15); LC-MS (Method IV): Rₜ=1.19 min; MS (ESIpos): m/z=434.

### Example 61

### 2-(4-Chloro-2,6-dimethylphenoxy)-4-[2,6-dioxo-4-(trifluoromethyl)-3,6-dihydropyrimidin-1(2H)-yl]-5-fluorobenzonitrile

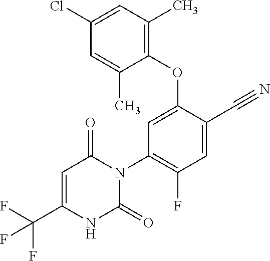 [SMILES: Cc1cc(Cl)cc(C)c1Oc1cc(-n2c(=O)cc(C(F)(F)F)[nH]c2=O)c(F)cc1C#N]

According to GP4, literature known 4-[2,6-dioxo-4-(trifluoromethyl)-3,6-dihydropyrimidin-1(2H)-yl]-2,5-difluorobenzonitrile (DE 10307142 A1 20040902, CAS: 162926-25-2, 100 mg, 315 μmol), commercially available 4-chloro-2,6-dimethylphenol (CAS: 1123-63-3, 49.4 mg, 315 μmol), and potassium carbonate (109 mg, 788 μmol) were heated in DMSO (2 mL) at 110° C. for 21 h. Upon reaction completion, the reaction mixture was filtered and purified using HPLC-HT to give the title compound (83 mg, 55%) as an light ochre solid. ¹H-NMR (400 MHz, DMSO-d₆) δ [ppm]: 2.074 (4.80), 2.079 (16.00), 2.518 (0.41), 2.539 (0.44), 6.359 (3.40), 6.917 (2.17), 6.931 (2.08), 7.333 (6.00), 8.162 (2.22), 8.185 (2.18); LC-MS (Method IV): Rₜ=1.22 min; MS (ESIneg): m/z=453.

### Example 62

### 4-[2,6-Dioxo-4-(trifluoromethyl)-3,6-dihydropyrimidin-1 (2H)-yl]-5-fluoro-2-(2,4,6-tri methylphenoxy)benzonitrile

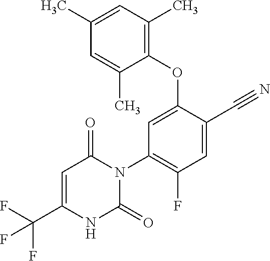 [SMILES: Cc1cc(C)c(Oc2cc(-n3c(=O)cc(C(F)(F)F)[nH]c3=O)c(F)cc2C#N)c(C)c1]

According to GP4, literature known 4-[2,6-dioxo-4-(trifluoromethyl)-3,6-dihydropyrimidin-1(2H)-yl]-2,5-difluorobenzonitrile (DE 10307142 A1 20040902, CAS: 162926-25-2, 100 mg, 315 μmol), commercially available 2,4,6-trimethylphenol (CAS: 527-60-6, 42.9 mg, 315 μmol), and potassium carbonate (109 mg, 788 μmol) were heated in DMSO (2 mL) at 110° C. for 21 h. Upon reaction completion, the reaction mixture was filtered and purified using HPLC-HT to give the title compound (46 mg, 55% as an light yellow solid. ¹H-NMR (400 MHz, DMSO-d₆) δ [ppm]: 2.042 (16.00), 2.074 (5.39), 2.084 (0.54), 2.254 (8.50), 6.338 (3.40), 6.833 (2.06), 6.848 (2.02), 7.006 (4.81), 8.131 (2.25), 8.154 (2.24). LC-MS (Method IV): Rt=1.21 min; MS (ESIneg): m/z=434.

### Example 63

### 2-(2,6-Difluorophenoxy)-4-[2,6-dioxo-4-(trifluoromethyl)-3,6-dihydropyrimidin-1 (2H)-yl]-5-fluorobenzonitrile

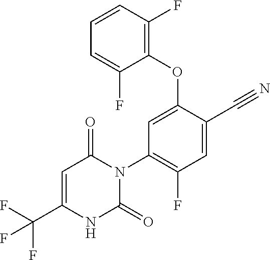 [SMILES: N#Cc1cc(F)c(-n2c(=O)cc(C(F)(F)F)[nH]c2=O)cc1Oc1c(F)cccc1F]

According to GP4, literature known 4-[2,6-dioxo-4-(trifluoromethyl)-3,6-dihydropyrimidin-1(2H)-yl]-2,5-difluorobenzonitrile (DE 10307142 A1 20040902, CAS: 162926-25-2, 100 mg, 315 μmol), commercially available 2,6-difluorophenol (CAS: 28177-48-2, 41.0 mg, 315 μmol), and potassium carbonate (109 mg, 788 μmol) were heated in DMSO (2 mL) at 110° C. for 21 h. Upon reaction completion, the reaction mixture was filtered and purified using HPLC-HT to give the title compound (12 mg, 8%) as an ochre brown solid. ¹H-NMR (400 MHz, DMSO-d₆) δ [ppm]: 2.074 (4.36), 2.518 (3.69), 2.523 (2.61), 2.539 (6.28), 3.351 (2.20), 6.390 (16.00), 7.338 (6.60), 7.353 (6.67), 7.365 (1.52), 7.369 (3.38), 7.374 (4.38), 7.380 (1.26), 7.392 (10.43), 7.404 (1.32), 7.414 (9.44), 7.419 (2.78), 7.428 (2.56), 7.444 (5.38), 7.452 (1.39), 7.458 (2.44), 7.464 (3.40), 7.469 (1.24), 7.476 (1.52), 7.485 (1.86), 7.501 (0.81), 8.221 (11.41), 8.243 (11.56); LC-MS (Method IV): Rₜ=1.03 min; MS (ESIpos): m/z=426 [M+H]⁺.

In a similar fashion, the following compounds were synthesized:

TABLE 5

| Example No | Structure, IUPAC-Name and analytics |
|---|---|
| 64 | 2-(2-Amino-6-fluorophenoxy)-4-[2,6-dioxo-4-(trifluoromethyl)-3,6- |
| | dihydropyrimidin-1(2H)-yl]-5-fluorobenzonitrile |
| | 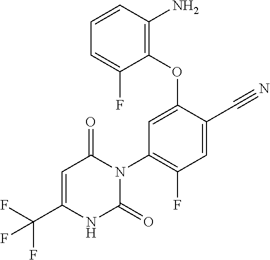 [SMILES: N#Cc1cc(F)c(-n2c(=O)cc(C(F)(F)F)[nH]c2=O)cc1Oc1c(N)cccc1F] |
| | LC-MS (Method I): Rₜ = 1.02 min; MS (ESIpos): m/z = 425 [M + H]⁺. |
| 65 | 2-(2-Amino-6-methylphenoxy)-4-[2,6-dioxo-4-(trifluoromethyl)-3,6- |
| | dihydropyrimidin-1(2H)-yl]-5-fluorobenzonitrile |
| | 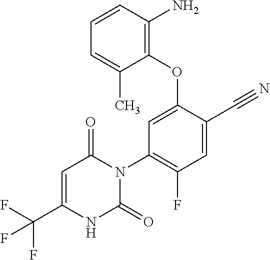 [SMILES: Cc1cccc(N)c1Oc1cc(-n2c(=O)cc(C(F)(F)F)[nH]c2=O)c(F)cc1C#N] |
| | LC-MS (Method I): Rₜ = 1.05 min; MS (ESIpos): m/z = 421 [M + H]⁺. |
| 66 | 2-(2,4-Dimethylphenoxy)-4-[2,6-dioxo-4-(trifluoromethyl)-3,6- |
| | dihydropyrimidin-1(2H)-yl]-5-fluorobenzonitrile |
| | 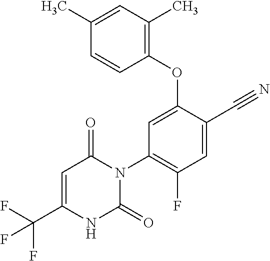 [SMILES: Cc1ccc(Oc2cc(-n3c(=O)cc(C(F)(F)F)[nH]c3=O)c(F)cc2C#N)c(C)c1] |
| | LC-MS (Method 1): Rₜ = 1.24 min; MS (ESIpos): m/z = 419 [M + H]⁺. |
| 67 | 2-(2,3-Dimethylphenoxy)-4-[2,6-dioxo-4-(trifluoromethyl)-3,6- |
| | dihydropyrimidin-1(2H)-yl]-5-fluorobenzonitrile |
| | 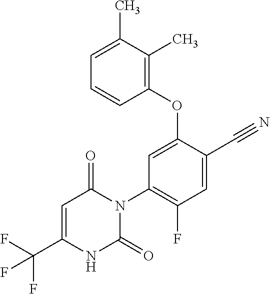 [SMILES: Cc1cccc(Oc2cc(-n3c(=O)cc(C(F)(F)F)[nH]c3=O)c(F)cc2C#N)c1C] |
| | ¹H-NMR (400 MHz, DMSO-d₆) δ [ppm]: 2.174 (16.00), 2.327 (1.28), 2.331 |
| | (0.98), 2.336 (0.41), 2.518 (4.27), 2.523 (3.09), 2.539 (0.85), 2.669 (1.28), |
| | 2.673 (0.89), 6.141 (10.01), 7.061 (3.20), 7.076 (3.15), 7.088 (2.01), 7.091 |
| | (2.06), 7.108 (2.40), 7.111 (2.38), 7.202 (0.87), 7.205 (0.91), 7.221 (2.31), |
| | 7.223 (2.22), 7.239 (1.65), 7.242 (1.44), 7.288 (1.14), 7.291 (1.30), 7.306 |
| | (1.58), 7.311 (1.71), 7.327 (0.69), 7.330 (0.66), 7.376 (1.87), 7.378 (1.81), |
| | 7.395 (1.51), 7.875 (2.93), 7.896 (5.30), 7.942 (5.12), 7.963 (2.70), 8.161 |
| | (3.66), 8.183 (3.66), 11.861 (0.55); LC-MS (Method I): Rₜ = 0.84 min; MS |
| | (ESIneg): m/z = 480 [M − H]⁻; LC-MS Method A): Rₜ = 1.19 min; MS (ESIpos): |
| | m/z = 420 [M + H]⁺. |

### Example 68

### 4-{2,6-Dioxo-4-[4-(trifluoromethyl)phenyl]-3,6-dihydropyrimidin-1(2H)-yl}-5-fluoro-2-(2-methylphenoxy)benzonitrile

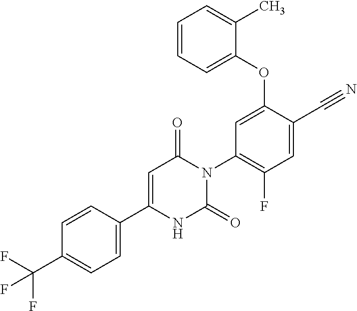 [SMILES: Cc1ccccc1Oc1cc(-n2c(=O)cc(-c3ccc(C(F)(F)F)cc3)[nH]c2=O)c(F)cc1C#N]

According to GP2, to a suspension of sodium hydride (13.7 mg, 344 μmol, 60% in mineral oil) in DMF (600 NL) was added commercially available ethyl (2E)-3-amino-3-[4-(trifluoromethyl)phenyl]prop-2-enoate (CAS: 68210-92-4, 83.1 mg, 321 μmol) at 0° C. After stirring at this temperature for 40 min, ethyl [4-cyano-2-fluoro-5-(2-methylphenoxy)phenyl]carbamate (INT-22, 80 mg, 229 μmol) was added. The resulting mixture was stirred for 2 days at 100° C. under nitrogen atmosphere. After cooling to room temperature, the reaction was poured into water (10 mL). To the aqueous phase, 2 M aq. HCl (1 mL) was slowly added until the pH reached 3 and the resulting precipitate was filtered off (starting material). The corresponding residue was dissolved in DMSO, filtered and purified using H PLC-HT giving the title compound as a colourless solid (24.8 mg, 21%).

### Example 69

### 4-{2,6-Dioxo-4-[4-(trifluoromethyl)phenyl]-3,6-dihydropyrimidin-1(2H)-yl}-5-methoxy-2-(2-methylphenoxy)benzonitrile (rac)

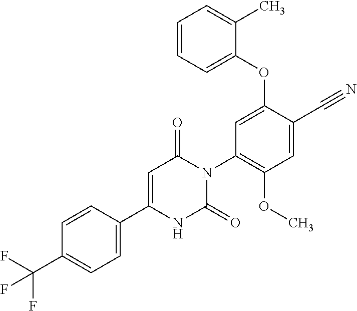 [SMILES: COc1cc(C#N)c(Oc2ccccc2C)cc1-n1c(=O)cc(-c2ccc(C(F)(F)F)cc2)[nH]c1=O]

A mixture of aforementioned 4-(4-bromo-2,6-dioxo-3,6-dihydropyrimidin-1(2H)-yl)-5-methoxy-2-(2 methylphenoxy)benzonitrile (Example 19, 100 mg, 234 μmol), commercially available [4-(trifluoromethyl)phenyl]boronic acid (CAS: 128796-39-4, 44.4 mg, 234 μmol) and commercially available potassium fluoride (CAS: 7789-23-3, 40.7 mg, 701 μmol) in dioxane (970 μL) were stirred under argon atmosphere at room temperature. Then, commercially available tris(dibenzylideneacetone)dipalladium(0) (CAS: 51364-51-3, 10.7 mg, 11.7 μmol, 0.05 eq.) and commercially available tri-tert-butylphosphoniumtetrafluoroboronate (CAS: 131274-22-1, 6.77 mg, 23.4 μmol) were added to the reaction mixture which was heated to 110° C. for 18 h. Upon reaction completion, the reaction mixture was purified using HPLC-HT to give the title compound as a light yellow solid (38 mg, 30%). ¹H-NMR (400 MHz, DMSO-d₆) δ [ppm]: 2.182 (0.43), 2.203 (16.00), 2.518 (1.43), 2.522 (0.88), 2.539 (0.44), 3.328 (1.84), 3.794 (0.50), 6.095 (4.04), 6.098 (4.06), 6.954 (2.09), 6.956 (2.19), 6.970 (10.59), 7.141 (0.93), 7.144 (0.95), 7.160 (2.38), 7.162 (2.30), 7.179 (1.58), 7.181 (1.49), 7.243 (1.09), 7.247 (1.24), 7.262 (1.63), 7.267 (1.75), 7.282 (0.72), 7.285 (0.70), 7.344 (1.89), 7.362 (1.62), 7.749 (8.26), 7.872 (2.99), 7.893 (5.21), 7.945 (5.07), 7.966 (2.79), 11.710 (2.64); LC-MS (Method I): Rₜ=1.25 min; MS (ESIpos): m/z=494 [M+H]⁺.

### Example 70

### 4-[4-(3-Chlorophenyl)-2,6-dioxo-3,6-dihydropyrimidin-1(2H)-yl]-5-fluoro-2-(2-methylphenoxy)benzonitrile

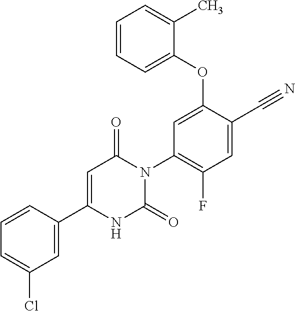 [SMILES: Cc1ccccc1Oc1cc(-n2c(=O)cc(-c3cccc(Cl)c3)[nH]c2=O)c(F)cc1C#N]

A mixture of aforementioned 4-(4-Bromo-2,6-dioxo-3,6-dihydropyrimidin-1(2H)-yl)-5-methoxy-2-(2 methylphenoxy)benzonitrile (Example 3, 100 mg, 240 μmol), commercially available 3-chlorophenyl)boronic acid (CAS: 63503-60-6, 37.6 mg, 240 μmol) and commercially available potassium fluoride (CAS: 7789-23-3, 41.9 mg, 721 μmol) in dioxane (1 mL) were stirred under argon atmosphere at room temperature. Then, commercially available tris(dibenzylideneacetone)dipalladium(0) (CAS: 51364-51-3, 11.0 mg, 12.0 μmol, 0.05 eq.) and commercially available tri-tert-butylphosphoniumtetrafluoroboronate (CAS: 131274-22-1, 6.97 mg, 24.0 μmol)) were added to the reaction mixture which was heated to 110° C. for 18 h. Upon reaction completion, the reaction mixture was purified using HPLC-HT to give the title compound as a light brown solid (14 mg, 12%). ¹H-NMR (400 MHz, DMSO-d₆) δ [ppm]: 2.074 (0.96), 2.154 (1.05), 2.173 (16.00), 2.518 (0.89), 2.523 (0.60), 6.127 (7.55), 7.053 (3.71), 7.068 (3.73), 7.084 (1.99), 7.088 (2.08), 7.105 (2.45), 7.107 (2.39), 7.200 (0.93), 7.203 (0.96), 7.219 (2.50), 7.222 (2.34), 7.237 (1.79), 7.240 (1.59), 7.287 (1.22), 7.290 (1.34), 7.306 (1.71), 7.309 (1.74), 7.325 (0.73), 7.328 (0.67), 7.374 (2.00), 7.376 (1.89), 7.393 (1.63), 7.517 (1.60), 7.537 (3.86), 7.557 (2.86), 7.613 (1.82), 7.616 (2.20), 7.618 (2.06), 7.621 (2.01), 7.634 (1.24), 7.636 (1.27), 7.639 (1.45), 7.641 (1.22), 7.696 (1.66), 7.699 (1.97), 7.701 (2.04), 7.703 (1.67), 7.716 (1.36), 7.720 (1.72), 7.723 (1.31), 7.836 (2.48), 7.840 (4.29), 7.844 (2.28), 8.157 (3.93), 8.180 (3.82), 11.730 (1.95); LC-MS (Method I): Rₜ=1.25 min; MS (ESIpos): m/z=448 [M+H]⁺.

### Example 71

### 1-[4-Cyano-2-fluoro-5-(2-methylphenoxy)phenyl]-2,6-dioxo-N-(2,2,2-trifluoroethyl)-1,2,3,6-tetrahydropyrimidine-4-carboxamide

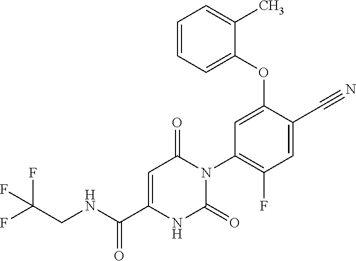 [SMILES: Cc1ccccc1Oc1cc(-n2c(=O)cc(C(=O)NCC(F)(F)F)[nH]c2=O)c(F)cc1C#N]

1-[4-Cyano-2-fluoro-5-(2-methylphenoxy)phenyl]-2,6-dioxo-1,2,3,6-tetrahydropyrimidine-4-carboxylic acid (INT-49, 60.0 mg, 157 μmol) and commercially available 2,2,2-trifluoroethan-1-amine (CAS: 753-90-2, 14 μL, 170 μmol, 1.1 eq.) were solved in ethylacetate and 0.5 mL DMF. Then commercially available T3P (CAS: 68957-94-8, 240 mg, 378 μmol, 2.4 eq.) and DIPEA (82 μL, 470 μmol) were added and the mixture was stirred under argon atmosphere at room temperature for 3 days. Upon reaction completion, water was added and the layers were separated. The aqueous layer was extracted three times with ethyl acetate. The combined organic layers were washed with brine, dried over sodium sulfate, filtered and evaporated to give crude material which was directly dissolved in DMSO and purified by HPLC-HT to give the title compound (13.0 mg, 17%) as a white solid. ¹H-NMR (400 MHz, DMSO-d₆) δ [ppm]: 1.237 (0.67), 2.151 (16.00), 2.331 (1.36), 2.518 (7.09), 2.523 (4.49), 2.539 (0.50), 2.673 (1.40), 4.054 (0.96), 4.070 (1.46), 4.079 (1.03), 4.086 (1.06), 4.094 (1.33), 4.110 (0.93), 6.285 (6.25), 7.051 (2.89), 7.066 (3.33), 7.089 (2.46), 7.092 (2.43), 7.200 (0.93), 7.215 (2.36), 7.218 (2.30), 7.233 (1.70), 7.236 (1.53), 7.282 (1.33), 7.303 (1.76), 7.318 (0.70), 7.367 (1.90), 7.386 (1.56), 8.141 (3.76), 8.163 (3.69), 9.501 (0.77), 9.516 (1.60), 9.532 (0.77), 11.540 (2.43); LC-MS (Method IV): Rₜ=1.11 min; MS (ESIpos): m/z=463.

### Example 72

### 4-(4-Cyclopropyl-2,6-dioxo-3,6-dihydropyrimidin-1(2H)-yl)-5-fluoro-2-(2-methylphenoxy)benzonitrile

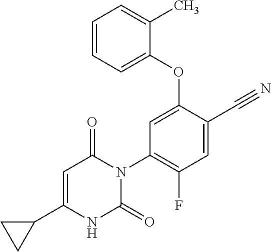 [SMILES: Cc1ccccc1Oc1cc(-n2c(=O)cc(C3CC3)[nH]c2=O)c(F)cc1C#N]

The aforementioned 4-(4-Bromo-2,6-dioxo-3,6-dihydropyrimidin-1(2H)-yl)-5-fluoro-2-(2-methylphenoxy)benzonitrile (Example 3, 50.0 mg, 120 μmol), commercially available potassium cyclopropyl(trifluorido)borate (19.6 mg, 132 μmol), commercially available palladium(II)-acetate (1.42 mg, 2.40 μmol), potassium carbonate 33.2 mg, 240 μmol) and commercially available tricyclohexylphosphine (CAS: 2622-14-2, 1.35 mg, 4.81 μmol) were suspended in toluene (1 mL) and water (40 μL) under argon atmosphere. The mixture was flushed with argon several times. After heating the reaction mixture for 18 h to 90° C., the reaction was not complete. Additional portions of potassium cyclopropyl(trifluorido)borate (1−) (1.1 eq.) and tricyclohexylphosphine (0.04 eq.) and palladium(ii)-acetate (0.02 eq) were added and the mixture was stirred for additional 6 h at 90° C. Upon reaction completion, the mixture was filtered and the residue was washed with ethyl acetate, filtered off, and the filtrate was evaporated to give crude material which was directly dissolved in DMSO and purified using HPLC-HT to give the title compound as a colourless solid (4 mg, 10%). ¹H-NMR (400 MHz, CHLOROFORM-d) δ [ppm]:—0.008 (1.27), 0.008 (1.23), 0.060 (0.56), 0.069 (16.00), 0.077 (1.07), 0.078 (1.07), 1.575 (0.43), 1.596 (0.42), 1.641 (0.43), 2.222 (7.17), 2.638 (0.41), 6.068 (4.96), 6.576 (1.63), 6.591 (1.63), 7.017 (0.82), 7.020 (0.84), 7.037 (1.02), 7.040 (1.02), 7.168 (0.85), 7.172 (0.90), 7.186 (0.74), 7.190 (0.66), 7.213 (0.45), 7.217 (0.58), 7.232 (0.60), 7.237 (0.71), 7.273 (0.86), 7.291 (0.56), 7.515 (1.70), 7.536 (1.67); LC-MS (Method III): Rₜ=1.09 min; MS (ESIneg): m/z=377.

### Example 73

### 1-[4-Cyano-2-methoxy-5-(2-methylphenoxy)phenyl]-2,6-dioxo-1,2,3,6-tetrahydropyrimidine-4-carboxamide (rac)

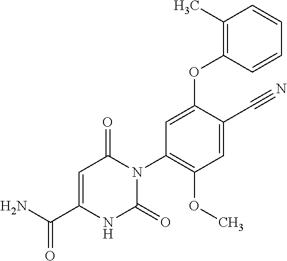 [SMILES: COc1cc(C#N)c(Oc2ccccc2C)cc1-n1c(=O)cc(C(N)=O)[nH]c1=O]

The aforementioned 4-(4-bromo-2,6-dioxo-3,6-dihydropyrimidin-1(2H)-yl)-5-methoxy-2-(2 methylphenoxy)benzonitrile (Example 19, 70.0 mg, 163 μmol), commercially available catalyst Palladium(π-cinnamyl) chloride dimer (CAS: 12131-44-1, 4.23 mg, 8.2 μmol), commercially available 1,1′-bis(diphenylphosphino)ferrocene (CAS: 12150-46-8, 4.53 mg, 8.17 μmol), commercially available zinc cyanide (CAS: 557-21-1, 19.2 mg, 163 μmol,) were placed in a 5 mL crimp sealable reaction vessel, sealed and flushed with argon. N,N-diisopropylethylamine (57 μL, 330 μmol) and degassed N,N-dimethylacetamide (1 ml) were added, and the mixture stirred for 14 h at 120° C. Saturated aqueous sodium bicarbonate solution was added and the mixture was extracted three times with dichloromethane. The combined organic layers were filtered using a water-repellent filter and concentrated in vacuo. The residue was purified by reverse phase HPLC, yielding the title compound (32.8 mg, 51% yield), which turned out to be a carboxamide instead of the initially expected nitrile. ¹H-NMR (600 MHz, DMSO-d₆) δ [ppm]: 0.000 (4.45), 1.959 (0.23), 2.189 (16.00), 2.384 (0.38), 2.388 (0.53), 2.391 (0.38), 2.518 (1.59), 2.522 (1.64), 2.525 (1.32), 2.543 (15.82), 2.612 (0.39), 2.616 (0.54), 2.619 (0.38), 2.785 (0.21), 2.945 (0.27), 3.342 (14.29), 6.213 (7.10), 6.927 (2.21), 6.940 (2.40), 6.949 (6.61), 7.140 (0.98), 7.142 (0.97), 7.152 (2.27), 7.154 (2.24), 7.165 (1.39), 7.238 (1.02), 7.240 (1.07), 7.251 (1.68), 7.253 (1.70), 7.264 (0.78), 7.266 (0.75), 7.337 (1.86), 7.349 (1.66), 7.719 (7.44), 8.046 (1.86), 8.290 (2.02), 8.319 (0.33), 11.098 (0.81); LC-MS (Method I): Rₜ=0.94 min; MS (ESIneg): m/z=391 [M−H]⁻.

### Example 74

### 4-[4-(Benzenesulfonyl)-2,6-dioxo-3,6-dihydropyrimidin-1(2H)-yl]-5-fluoro-2-(2-methylphenoxy)benzonitrile

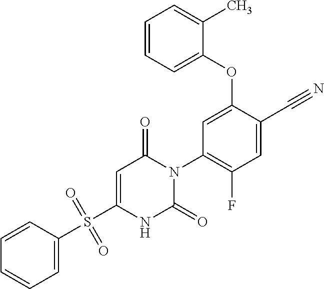 [SMILES: Cc1ccccc1Oc1cc(-n2c(=O)cc(S(=O)(=O)c3ccccc3)[nH]c2=O)c(F)cc1C#N]

The aforementioned 4-(4-Bromo-2,6-dioxo-3,6-dihydropyrimidin-1(2H)-yl)-5-fluoro-2-(2-methylphenoxy)benzonitrile (Example 3, 150 mg, 360 μmol), dissolved in DMSO (3 mL), was stirred with sodium benzenesulfinate (89.3 mg, 544 μmol), commercially available (1S,2S)-cyclohexane-1,2-diamine (CAS: 1121-22-8, 18 μL, 150 μmol, 0.41 eq) and commercially available copper (i) trifluoromethanesulfinate benzene complex (CAS: 42152-46-5; 20.2 mg, 36.0 μmol, 0.1 eq.) under argon atmosphere. The resulting reaction mixture was heated to 110° C. for 20 h. After reaction completion, the mixture was filtered. After two consecutive purifications using HPLC HT, the title compound was obtained as a light brown solid (7 mg, 4%). ¹H-NMR (400 MHz, DMSO-d₆) δ [ppm]: 1.235 (0.96), 2.075 (1.69), 2.121 (16.00), 2.327 (2.60), 2.332 (1.91), 2.522 (9.44), 2.664 (1.91), 2.669 (2.64), 2.673 (2.05), 3.361 (5.42), 6.380 (2.83), 6.995 (1.78), 7.009 (1.69), 7.057 (2.10), 7.077 (2.51), 7.181 (0.91), 7.200 (2.32), 7.216 (1.73), 7.263 (1.37), 7.282 (1.82), 7.300 (0.68), 7.347 (2.01), 7.364 (1.60), 7.710 (2.01), 7.729 (4.47), 7.749 (3.05), 7.831 (1.60), 7.849 (2.23), 7.868 (0.87), 8.094 (3.92), 8.116 (5.65), 8.138 (3.19; LC-MS (Method IV): Rₜ=1.18 min; MS (ESIpos): m/z=478.

### Example 75

### 5-Fluoro-4-[4-(methanesulfonyl)-2,6-dioxo-3,6-dihydropyrimidin-1(2H)-yl]-2-(2-methylphenoxy)benzonitrile

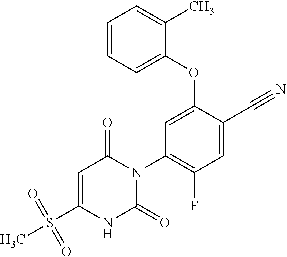 [SMILES: Cc1ccccc1Oc1cc(-n2c(=O)cc(S(C)(=O)=O)[nH]c2=O)c(F)cc1C#N]

The aforementioned 4-(4-Bromo-2,6-dioxo-3,6-dihydropyrimidin-1(2H)-yl)-5-fluoro-2-(2-methylphenoxy)benzonitrile (Example 3, 100 mg, 240 μmol, 1.0 eq.) was dissolved in DMSO (2 mL) under argon atmosphere and commercially available copper catalyst copper(I) trifluoromethansulfonate benzene complex (CAS: 42152-46-5, 13.4 mg, 0 μmol, 0.1 eq.), commercially available sodium methanesulfinate (37.0 mg, 363 μmol, 1.5 eq.) and commercially available ligand (+/−)-trans-1,2-diaminocyclohexane (CAS: 1121-22-8, 12 μL, 99 μmol, 0.4 eq.) were added to the mixture. The reaction mixture was heated to 110° C. for 20 h. After cooling, the solvent was removed under vacuum. The residue was purified by HPLC-HT giving the title compound (36 mg, 34%) as a light ochre solid. ¹H-NMR (400 MHz, DMSO-d₆) δ [ppm]: 2.147 (16.00), 2.459 (0.52), 2.518 (0.87), 2.523 (0.58), 2.539 (1.39), 6.212 (7.61), 7.028 (3.46), 7.042 (3.41), 7.095 (1.93), 7.098 (2.03), 7.115 (2.34), 7.118 (2.38), 7.207 (0.88), 7.210 (0.92), 7.226 (2.33), 7.228 (2.22), 7.244 (1.71), 7.247 (1.50), 7.289 (1.14), 7.293 (1.33), 7.308 (1.60), 7.313 (1.76), 7.328 (0.66), 7.331 (0.62), 7.375 (1.84), 7.377 (1.80), 7.394 (1.51), 8.160 (3.80), 8.182 (3.80). LC-MS (Method V): Rₜ=1.03 min; MS (ESIpos): m/z=415.

### Example 76

### 4-[4-(Benzenesulfonyl)-2,6-dioxo-3,6-dihydropyrimidin-1(2H)-yl]-5-methoxy-2-(2-methylphenoxy)benzonitrile (rac)

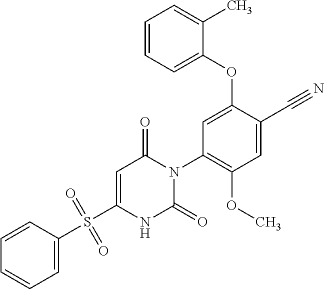 [SMILES: COc1cc(C#N)c(Oc2ccccc2C)cc1-n1c(=O)cc(S(=O)(=O)c2ccccc2)[nH]c1=O]

In a pressure vessel, the aforementioned 4-[4-bromo-2,6-dioxo-3,6-dihydropyrimidin-1(2H)-yl]-5-methoxy-2-(2-methylphenoxy)benzonitrile (Example 19, 500 mg, 1.17 mmol) was dissolved in DMSO (9.6 mL) under argon atmosphere. The commercially available copper catalyst copper (i) trifluoromethansulfonate benzene complex (CAS: 42152-46-5, 65.3 mg, 117 μmol), commercially available sodium benzenesulfinate (CAS: 873-55-2, 289 mg, 1.76 mmol) and the commercially available ligand (+/−)-trans-1,2-diaminocyclohexane (CAS: 1121-22-8, 57 μL, 480 μmol) were added and the reaction mixture was heated to 110° C. for 20 h. After cooling, the reaction mixture was filtered and purified by HPLC giving the title compound as a dark yellow solid (113 mg, 19%). ¹H-NMR (400 MHz, DMSO-d₆) δ [ppm]: 2.074 (1.79), 2.149 (16.00), 2.169 (0.77), 2.518 (1.02), 2.522 (0.63), 2.539 (1.01), 3.775 (1.04), 6.372 (6.84), 6.932 (2.18), 6.950 (2.42), 6.952 (2.43), 6.974 (8.24), 7.124 (0.89), 7.126 (0.99), 7.142 (2.32), 7.144 (2.31), 7.160 (1.61), 7.163 (1.51), 7.217 (1.13), 7.221 (1.28), 7.236 (1.64), 7.240 (1.79), 7.255 (0.74), 7.259 (0.72), 7.315 (1.91), 7.317 (1.90), 7.333 (1.61), 7.704 (8.11), 7.724 (2.17), 7.743 (4.61), 7.763 (3.32), 7.845 (0.98), 7.847 (1.74), 7.851 (1.10), 7.862 (0.89), 7.866 (2.56), 7.882 (0.61), 7.885 (0.96), 8.118 (3.64), 8.121 (4.83), 8.140 (4.00); LC-MS (Method III): Rₜ=1.18 min; MS (ESIpos): m/z=489 [M+H]⁺. Atropisomeric ratio: atrop 1/atrop 2=44:44 (12% impurities); Rₜ (atrop1)=2.39 min; Rₜ (atrop2)=2.85 min. The atropisomeric ratio was determined using the following chiral HPLC method: instrument: Waters Alliance 2695; column: Amylose SA 3 μm 100×4.6 mm; eluent A: 2-methoxy-2-methylpropane+0.1 vol % diethylamine (99%); eluent B: methanol; isocratic: 50% A+50% B; flow: 1.4 mL/min; T: 25° C.; DAD: 254 nm.

The racemate was separated into its single atropisomers using preparative chiral HPLC: instrument: PrepCon Labomatic HPLC; column: YMC Amylose SA 5 μm 250×30; eluent A: hexane+0.1% diethylamine; eluent B: ethanol; gradient 20 to 50% B in 20 min; flow: 50 mL/min; temperature: 25° C.; UV 254 nm.

### Example 77

### 4-[4-(Benzenesulfonyl)-2,6-dioxo-3,6-dihydropyrimidin-1(2H)-yl]-5-methoxy-2-(2-methylphenoxy)benzonitrile (atrop 1)

58 mg; light yellow solid, [α]D=−21.4°+/−0.87°.

### Example 78

### 4-[4-(Benzenesulfonyl)-2,6-dioxo-3,6-dihydropyrimidin-1(2H)-yl]-5-methoxy-2-(2-methylphenoxy)benzonitrile (atrop 2)

57 mg; light yellow solid, [α]D=+29.6°+/−0.95°.

### Example 79

### 4-[4-(Methanesulfonyl)-2,6-dioxo-3,6-dihydropyrimidin-1(2H)-yl]-5-methoxy-2-(2-methylphenoxy)benzonitrile (rac)

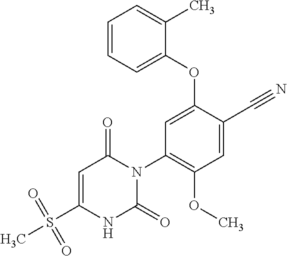 [SMILES: COc1cc(C#N)c(Oc2ccccc2C)cc1-n1c(=O)cc(S(C)(=O)=O)[nH]c1=O]

In a pressure vessel the aforementioned 4-[4-bromo-2,6-dioxo-3,6-dihydropyrimidin-1(2H)-yl]-5-methoxy-2-(2-methylphenoxy)benzonitrile (Example 19, 100 mg, 234 μmol) was dissolved in DMSO (1.9 mL) under argon atmosphere. The commercially available copper catalyst copper (i) trifluoromethansulfonate benzene complex (CAS: 42152-46-5, 13.1 mg, 23.4 μmol), commercially available sodium methanesulfinate (CAS: 20277-69-4, 36.0 mg, 353 μmol) and the commercially available ligand (+/−)-trans-1,2-diaminocyclohexane (CAS: 1121-22-8, 11 μL, 96 μmol) were added and the reaction mixture was heated to 110° C. for 20 h. After cooling, the reaction mixture was filtered and purified by HPLC giving the title compound as a light yellow solid (42 mg, 38%). ¹H-NMR (400 MHz, DMSO-d₆) δ [ppm]: 2.174 (12.22), 2.518 (0.83), 2.523 (0.52), 2.539 (1.18), 3.336 (0.85), 3.796 (16.00), 6.162 (1.47), 6.929 (4.10), 6.964 (1.57), 6.966 (1.64), 6.984 (1.80), 6.987 (1.81), 7.148 (0.69), 7.150 (0.73), 7.166 (1.79), 7.169 (1.75), 7.184 (1.22), 7.188 (1.14), 7.245 (0.83), 7.248 (0.94), 7.264 (1.24), 7.268 (1.35), 7.284 (0.54), 7.287 (0.52), 7.342 (1.43), 7.344 (1.40), 7.360 (1.20), 7.740 (5.84); LC-MS (Method I): Rₜ=1.00 min; MS (ESIpos): m/z=428 [M+H]⁺. Atropisomeric ratio: atrop 1/atrop 2=50:49 (1% impurities); Rₜ (atrop1)=4.99 min; Rₜ (atrop2)=6.25 min. The atropisomeric ratio was determined using the following chiral HPLC method: instrument: Waters Alliance 2695 Agilent HPLC 1260; column: Chiralpak IE 3 μm 100×4.6 mm; eluent A: hexane+0.1 Vol-% TFA (99%); eluent B: 2-propanol; isocratic: 20% B; flow: 1.4 mL/min; T: 25° C.; DAD 254 nm. The racemate was separated into its single atropisomers using preparative chiral HPLC: Instrument: Labomatic HD5000, Labocord-5000; Gilson GX-241, Labcol Vario 4000, column: Chiralpak IE 5 μm 250×30 mm; eluent A: hexane+0.1 Vol-% TFA (99%); eluent B: 2-propanol; isocratic: 50% A+50% B; flow 40.0 mL/min; UV 254 nm.

### Example 80

### 4-[4-(Methanesulfonyl)-2,6-dioxo-3,6-dihydropyrimidin-1(2H)-yl]-5-methoxy-2-(2-methylphenoxy)benzonitrile (atrop 1)

16 mg; colourless solid, [α]D=+15.7°+/−2.61°.

### Example 81

### 4-[4-(Methanesulfonyl)-2,6-dioxo-3,6-dihydropyrimidin-1(2H)-yl]-5-methoxy-2-(2-methylphenoxy)benzonitrile (atrop 2)

16 mg; colourless solid, [α]D=−11.9°+/−1.68°.

### EXPERIMENTAL SECTION—ALTERNATIVE INTERMEDIATES & EXAMPLES

### Alternative INT-1

### Ethyl (5-Bromo-2-methoxyphenyl)carbamate

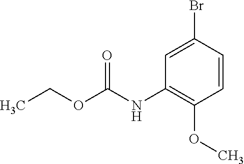

According to GP1, to a solution of commercially available 5-bromo-2-methoxyaniline (CAS: 6358-77-6, 500 mg, 2.47 mmol) in pyridine (3.7 mL) was added ethyl carbonochloridate (280 μL, 3.0 mmol) and the resulting mixture was stirred at room temperature overnight. Upon completion of the reaction, an aq. solution of HCl (1 M, 20 mL) was added resulting in the formation of a precipitate which was collected by filtration, and which was then washed with water and dried to give the desired intermediate (513 mg, 72%) as a light brown solid. ¹H-NMR (400 MHz, DMSO-d₆) δ [ppm]: 1.208 (7.49), 1.225 (16.00), 1.243 (7.65), 3.332 (4.35), 4.088 (2.40), 4.105 (7.40), 4.123 (7.28), 4.140 (2.30), 6.962 (4.20), 6.984 (5.04), 7.192 (2.93), 7.199 (2.97), 7.214 (2.39), 7.220 (2.36), 7.874 (2.50), 7.879 (2.44), 8.579 (3.01); LC-MS (method I): Rₜ=1.27 min; MS (ESIpos): m/z=273 [M+H]+

### Alternative INT-2

### 3-(5-Bromo-2-methoxyphenyl)-6-(trifluoromethyl)pyrimidine-2,4(1H,3H)-dione

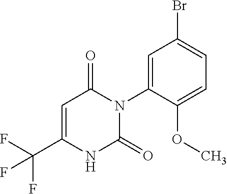 [SMILES: COc1ccc(Br)cc1-n1c(=O)cc(C(F)(F)F)[nH]c1=O]

According to GP2, to a suspension of sodium hydride (112 mg, 60% purity, 2.79 mmol) in DMF (4.9 mL), commercially available ethyl 3-amino-4,4,4-trifluorobut-2-enoate (CAS: 372-29-2, 380 μL, 2.6 mmol) and ethyl (5-bromo-2-methoxyphenyl)carbamate (alternative INT-1, 510 mg, 1.86 mmol) were added, and the mixture was stirred at 100° C. overnight. Upon completion of the reaction, aq. ammonium chloride solution was added at 0° C. to stop the reaction. The solvent was removed in vacuo and the residue was purified by HPLC-HT to give the title compound as a light brown solid (42 mg, 6%). ¹H-NMR (400 MHz, DMSO-d₆) δ [ppm]: 2.327 (0.52), 2.518 (1.95), 2.523 (1.24), 2.668 (0.76), 2.729 (0.66), 2.888 (0.79), 3.335 (2.35), 3.831 (0.55), 6.363 (16.00), 6.947 (0.54), 7.134 (11.25), 7.157 (12.15), 7.542 (12.10), 7.549 (14.76), 7.592 (8.62), 7.599 (6.84), 7.614 (7.34), 7.621 (6.28). LC-MS (method I): Rₜ=0.97 min; MS (ESIneg): m/z=363 [M−H]⁻.

### Alternative INT-3

### Ethyl (5-bromo-2-fluoro-4-methylphenyl)carbamate

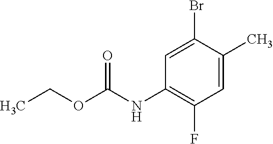

According to GP1, to a solution of commercially available 5-bromo-2-fluoro-4-methylaniline (CAS: 945244-29-1, 550 mg, 2.70 mmol) in pyridine (4.1 mL) was added ethyl carbonochloridate (310 μL, 3.2 mmol) and the resulting mixture was stirred at room temperature overnight. Upon completion of the reaction, an aq. solution of HCl (1 M, 20 mL) was added resulting in the formation of a precipitate which was collected by filtration, and which was then washed with water and dried to give the desired intermediate as a brown solid (650 mg, 70%). ¹H-NMR (400 MHz, DMSO-d₆) δ [ppm]: 1.140 (0.60), 1.210 (5.81), 1.228 (12.54), 1.237 (0.81), 1.246 (6.05), 2.266 (0.46), 2.286 (16.00), 2.518 (0.70), 2.523 (0.52), 4.093 (2.13), 4.111 (6.72), 4.117 (0.49), 4.128 (6.68), 4.146 (2.12), 4.152 (0.45), 7.270 (2.36), 7.299 (2.40), 7.861 (0.99), 7.880 (1.01), 9.423 (1.21); LC-MS (method I): Rₜ=1.27 min; MS (ESIneg): m/z=274 [M−H]⁻

### Alternative INT-4

### 3-(5-Bromo-2-fluoro-4-methyl phenyl)-6-(trifluoromethyppyrimidine-2,4(1H,3H)-dione

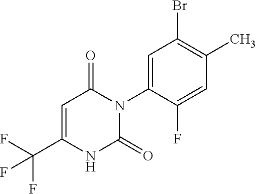 [SMILES: Cc1cc(F)c(-n2c(=O)cc(C(F)(F)F)[nH]c2=O)cc1Br]

According to GP2, to a suspension of sodium hydride (130 mg, 60% purity, 3.26 mmol)) in DMF (5.7 mL), commercially available ethyl 3-amino-4,4,4-trifluorobut-2-enoate (CAS: 372-29-2, 450 μL, 3.0 mmol) and ethyl (5-bromo-2-fluoro-4-methylphenyl)carbamate (450 μL, 3.0 mmol) (alternative INT-3, 600 mg, 2.17 mmol) were added, and the mixture was at 100° C. overnight. Upon completion of the reaction, aq. ammonium chloride solution was added at 0° C. to stop the reaction. The solvent was removed in vacuo and the residue was purified by HPLC-HT to give the title compound as a light brown solid (230 mg, 26%). ¹H-NMR (400 MHz, DMSO-d₆) δ [ppm]: 1.292 (0.66), 1.987 (0.61), 2.388 (16.00), 2.518 (0.84), 2.522 (0.55), 2.539 (4.29), 3.338 (0.61), 6.241 (1.62), 7.442 (2.30), 7.469 (2.32), 7.686 (2.06), 7.703 (2.06), 8.133 (3.49); LC-MS (method I): Rₜ=1.10 min; MS (ESIpos): m/z=367 [M+H]+

### Alternative INT-5

### Ethyl (5-bromo-2-chlorophenyl)carbamate

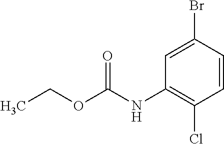

According to GP1, to a solution of commercially available 5-bromo-2-chloroaniline (CAS: 60811-17-8, 1.00 g, 4.84 mmol) in pyridine (20 mL) was added ethyl carbonochloridate (560 μL, 5.8 mmol) and the resulting mixture was stirred at room temperature overnight. Upon completion of the reaction, an aq. solution of HCl (1 M, 20 mL) was added resulting in the formation of a precipitate which was collected by filtration, and which was then washed with water and dried to give the desired intermediate (1.2 g, 80%, 80% purity) as a dark brown liquid. ¹H-NMR (400 MHz, DMSO-d₆) δ [ppm]: 1.112 (1.15), 1.130 (2.51), 1.148 (1.17), 1.171 (0.59), 1.222 (7.50), 1.240 (16.00), 1.258 (7.68), 1.986 (1.01), 2.518 (0.42), 4.109 (2.54), 4.126 (8.05), 4.144 (8.19), 4.162 (2.79), 7.337 (2.06), 7.343 (2.10), 7.359 (3.48), 7.365 (3.47), 7.421 (6.71), 7.443 (4.09), 7.569 (0.53), 7.839 (3.58), 7.845 (3.48), 7.895 (0.40), 9.203 (2.92); LC-MS (method I): Rₜ=1.32 min; MS (ESIpos): m/z=278 [M+H]+

### Alternative INT-6

### 3-(5-Bromo-2-chlorophenyl)-6-(trifluoromethyl)pyrimidine-2,4(1H,3H)-dione

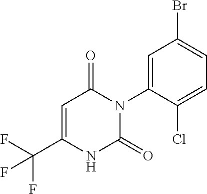 [SMILES: O=c1cc(C(F)(F)F)[nH]c(=O)n1-c1cc(Br)ccc1Cl]

According to GP2, to a suspension of sodium hydride (258 mg, 60% purity, 6.46 mmol)) in DMF (11 mL), commercially available ethyl 3-amino-4,4,4-trifluorobut-2-enoate (CAS: 372-29-2, 890 μL, 6.0 mmol) and ethyl (5-bromo-2-chlorophenyl)carbamate (alternative INT-5, 1.20 g, 4.31 mmol) were added, and the mixture was at 100° C. overnight. Upon completion of the reaction, aq. ammonium chloride solution was added at 0° C. to stop the reaction. The solvent was removed in vacuo and the residue was purified by HPLC-HT to give the title compound as a light brown solid (330 mg, 18%). ¹H-NMR (400 MHz, DMSO-d₆) δ [ppm]: 2.326 (0.97), 2.522 (2.90), 2.539 (1.34), 2.665 (0.75), 2.669 (0.95), 6.400 (5.65), 6.946 (1.39), 7.074 (1.42), 7.201 (1.39), 7.605 (10.20), 7.627 (16.00), 7.693 (8.70), 7.699 (9.05), 7.715 (5.29), 7.721 (5.83), 7.827 (9.08), 7.833 (8.29), 8.132 (1.81), 12.821 (0.84); LC-MS (method I): Rₜ=1.02 min; MS (ESIpos): m/z=369 [M+H]+

### Alternative Example 1

### 3-[2-Methoxy-5-(2-methylphenoxy)phenyl]-6-(trifluoromethyl)pyrimidine-2,4(1H,3H)-dione

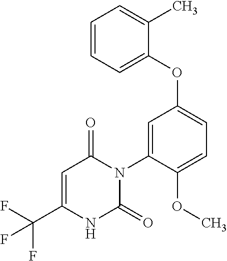 [SMILES: COc1ccc(Oc2ccccc2C)cc1-n1c(=O)cc(C(F)(F)F)[nH]c1=O]

According to GP3, 3-(5-bromo-2-methoxyphenyl)-6-(trifluoromethyl)pyrimidine-2,4(1H,3H)-dione (alternative INT-2, 37.0 mg, 101 μmol), commercially available 2-methylphenol (CAS: 95-48-7, 15 μL, 140 μmol), N,N-dimethylglycine (2.77 mg, 26.9 μmol), copper (I) iodide (2.61 mg, 13.7 μmol) and cesium carbonate (79.2 mg, 243 μmol) were heated in DMF (2 mL) for 18 h at 140° C. Upon reaction completion, work-up and purification the title compound was obtained as an ochre solid (9 mg, 19%). ¹H-NMR (400 MHz, DMSO-d₆) δ [ppm]: 2.024 (0.45), 2.047 (1.38), 2.207 (12.69), 2.322 (0.48), 2.326 (0.67), 2.331 (0.48), 2.522 (1.89), 2.664 (0.51), 2.669 (0.69), 2.673 (0.53), 3.718 (16.00), 6.305 (1.69), 6.830 (1.77), 6.849 (1.91), 6.938 (1.94), 6.944 (2.51), 6.984 (1.65), 6.992 (1.22), 7.007 (2.01), 7.015 (1.67), 7.043 (0.78), 7.060 (1.79), 7.062 (1.78), 7.078 (1.16), 7.132 (3.18), 7.155 (2.48), 7.172 (0.94), 7.176 (0.95), 7.195 (1.38), 7.211 (0.64), 7.288 (1.51), 7.306 (1.28), 8.996 (0.53), 12.516 (0.44); LC-MS (method I): Rₜ=1.18 min; MS (ESIpos): m/z=393 [M+H]⁺.

### Alternative Example 2

### 3-[2-Fluoro-4-methyl-5-(2-methylphenoxy)phenyl]-6-(trifluoromethyl)pyrimidine-2,4(1H,3H)-dione

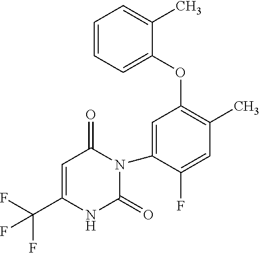 [SMILES: Cc1ccccc1Oc1cc(-n2c(=O)cc(C(F)(F)F)[nH]c2=O)c(F)cc1C]

According to GP3, 3-(5-bromo-2-fluoro-4-methylphenyl)-6-(trifluoromethyl)pyrimidine-2,4(1H,3H)-dione (alternative INT-4, 115 mg, 313 μmol), commercially available 2-methylphenol (CAS: 95-48-7, 48.1 mg, 445 μmol), N,N-dimethylglycine (8.56 mg, 83.0 μmol), copper (I) iodide (8.05 mg, 42.3 μmol) and cesium carbonate (245 mg, 752 μmol) were heated in DMF (6.2 mL) for 18 h at 140° C. Upon reaction completion, work-up and purification the title compound was obtained as brown solid (10 mg, 8%). ¹H-NMR (400 MHz, METHANOL-d₄) δ [ppm]: 2.252 (15.43), 2.316 (16.00), 2.656 (0.60), 6.211 (4.96), 6.680 (3.00), 6.697 (2.96), 6.754 (1.80), 6.756 (1.85), 6.774 (2.04), 6.777 (2.05), 6.991 (0.79), 6.994 (0.83), 7.010 (2.05), 7.012 (2.04), 7.028 (1.37), 7.031 (1.30), 7.102 (0.91), 7.105 (1.01), 7.121 (1.41), 7.125 (1.49), 7.141 (0.64), 7.143 (0.63), 7.211 (2.20), 7.236 (3.52), 7.251 (1.41); LC-MS (method I): Rₜ=1.28 min; MS (ESIpos): m/z=395 [M+H]⁺.

### Alternative Example 3

### 3-[2-Chloro-5-(2-methylphenoxy)phenyl]-6-(trifluoromethyl)pyrimidine-2,4(1H,3H)-dione

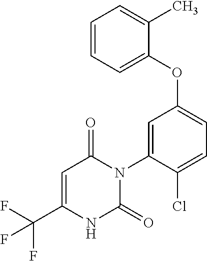 [SMILES: Cc1ccccc1Oc1ccc(Cl)c(-n2c(=O)cc(C(F)(F)F)[nH]c2=O)c1]

According to GP3, 3-(5-bromo-2-chlorophenyl)-6-(trifluoromethyl)pyrimidine-2,4(1H,3H)-dione (alternative INT-6, 150 mg, 406 μmol), commercially available 2-methylphenol (CAS: 95-48-7, 62.3 mg, 576 μmol), N,N-dimethylglycine (211.1 mg, 108 μmol), copper (I) iodide (10.4 mg, 54.8 μmol) and cesium carbonate (317 mg, 974 μmol) were heated in DMF (8 mL) for 18 h at 140° C. Upon reaction completion, work-up and purification the title compound was obtained as an ochre solid (60 mg, 35%). ¹H-NMR (400 MHz, DMSO-d₆) δ [ppm]: 2.154 (16.00), 2.518 (1.60), 2.523 (1.05), 6.385 (4.58), 6.996 (4.21), 7.003 (3.00), 7.017 (4.66), 7.025 (3.20), 7.093 (4.57), 7.101 (3.80), 7.144 (0.84), 7.147 (0.92), 7.163 (2.25), 7.166 (2.23), 7.181 (1.57), 7.185 (1.46), 7.248 (1.05), 7.251 (1.21), 7.266 (1.54), 7.271 (1.74), 7.287 (0.71), 7.290 (0.69), 7.340 (1.82), 7.341 (1.79), 7.359 (1.55), 7.575 (5.03), 7.597 (4.65), 12.671 (0.42); LC-MS (method G): Rₜ=1.22 min; MS (ESIpos): m/z=397 [M+H]+

### EXPERIMENTAL SECTION— BIOLOGICAL ASSAYS

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

The in vitro activity of the compounds of the present invention can be demonstrated in the following assays:

In Vitro Assay 1a and 1b: BCAT1 and BCAT2 Biochemical Assay

BOAT (Branched chain amino acid transferase) catalyzes transamination of the branched chain amino acid leucine to α-ketoisocaproate (KIC). The BOAT enzymatic reaction was measured in a coupled readout with leucine dehydrogenase (LeuDH) which catalyzes the Nicotinamide adenine dinucleotide (NADH) dependent reduction of KIC to leucine. NADH consumption is measured by fluorescent readout.

The biochemical reactions were performed at 32° C. in 384-well plates using a reaction volume of 51 μl and the following assay buffer conditions: 50 mM HEPES (2-[4-(2-hydroxyethyl)piperazin-1-yl]ethanesulfonic acid) pH 8.0, 50 mM aqueous solution of ammonium sulfate, 1 mM ethylenediaminetetraacetic acid, 2 mM dithiothreitol, 0.01% bovine serum albumine, 0.005% Brij 35 detergent, 0.5 μM PLP (pyridoxalphosphate), 10 μM NADH, and 50 μM α-ketoglutarate), and 100 μM leucine. The BOAT enzyme was used in a final concentration of 0.75 nM and the LeuDH enzyme in a final concentration of 0.1 nM. Test compounds were used in a concentration range between 0.005 and 30 μM. The final DMSO concentration was 2%.

The reaction was initiated by addition of substrates (Leucine, α-KG, NADH). Enzyme activity was monitored kinetically over 90 min by the consumption of NADH fluorescence (Extinction 340 nm/Emmission480 nm. Recombinant human BCAT1 enzyme (catalogue number ATGP1479) and recombinant human BCAT2 enzyme (catalogue no. ATGP1523) were purchased from ATGen (South Korea). Other chemicals were purchased from Sigma-Aldrich. Fluorescence measurements were obtained using a BMG clarion star plate-reading spectrophotometer. The decrease in fluorescence is proportional to BOAT activity. IC₅₀ values are determined by interpolation from plots of relative fluorescence versus inhibitor concentration.

In Vitro Assay 2: BCAT Cellular Assay

Levels of leucine were measured in medium of different tumor cell lines. The enzyme BOAT (Branched chain amino acid transferase) catalyzes the consumption of leucine.

Cells (U87-MG and MDA-MB231 from ATCC) were grown in Dulbecco's Modified Eagle Medium (DMEM) containing 10% fetal calf serum. They were harvested by trypsin and seeded into 96-well plates. Cells were incubated overnight at 37° C. in an atmosphere containing 5% carbon dioxide. The next day test compounds were added to each cell well. The final concentration of dimethylsulfoxide (DMSO) was 0.1% and DMSO controls were included. The plates were then placed in an incubator for 24 hours.

Leucine was measured in an enzymatic readout with leucine dehydrogenase (LeuDH) and diaphorase. Medium is deproteinized with perchloric acid. 30 μl of 1 M aqueous perchloric acid was added to each well and the plates were centrifuged. 5 μl of 3.2 M aqueous potassium carbonate solution was added to each well and the plates were centrifuged. 20 μl aliquots are removed and incubated with 40 μl buffer (50 mM Trisaminomethane pH 7.5, 0.5% glycerine, 0.01% Tween®, 0.05% bovine serum albumin with 0.125 U/mL LeuDH, 0.1 U/mL diaphorase, 1 mM NAD+, and 2 μM resazurin. The conversion of resazurin to resorufin was detected by fluorescence spectroscopy at extinction 540 nm and emission 600 nm. Chemicals and enzymes were purchased from Sigma-Aldrich. Fluorescence measurements were obtained using a BMG clarion star plate-reading spectrophotometer. The increase in fluorescence is proportional to leucine concentration. IC₅₀ values are determined by interpolation from plots of relative fluorescence versus inhibitor concentration.

TABLE 6
IC₅₀ values of examples in in vitro assays 1a and 1b

| Example | BCAT1 IC₅₀ [M] | BCAT2 IC₅₀ [M] |
|---|---|---|
| 1 | 3.40E-7 | 2,50E-6 |
| 2 | 4,00E-7 | 1.80E-6 |
| 3 | 3.15E-7 | 5.50E-6 |
| 4 | 1.62E-7 | 1.68E-6 |
| 5 | 1,13E-7 | 7,00E-7 |
| 6 | 6.25E-7 | 5,30E-6 |
| 7 | 2.87E-7 | 1,80E-6 |
| 8 | 1.10E-6 | 9,00E-6 |
| 9 | 8,00E-8 | 9,00E-7 |
| 10 | 3,90E-7 | 6,00E-6 |
| 11 | 1.20E-7 | 2,00E-6 |
| 12 | 7.50E-6 | n.d. |
| 13 | 1.35E-7 | 2,00E-6 |
| 14 | 1.43E-7 | 1.30E-6 |
| 15 | 1.48E-7 | 8.30E-7 |
| 16 | 1,50E-7 | 2.50E-6 |
| 17 | 1.90E-5 | n.d. |
| 18 | 2,00E-7 | 2.60E-6 |
| 19 | 2,00E-7 | 3,50E-6 |
| 20 | 2,35E-6 | 2.30E-5 |
| 21 | 1.45E-7 | 3,00E-6 |
| 22 | 1,50E-7 | n.d. |
| 23 | 1.80E-7 | n.d. |
| 24 | 1.45E-7 | 6,00E-7 |
| 25 | 2,00E-7 | 1.30E-6 |
| 26 | 2.70E-7 | 2,17E-6 |
| 27 | 4.38E-7 | 4.33E-6 |
| 28 | 2,80E-7 | 3.50E-6 |
| 29 | 2,00E-7 | 2,00E-7 |
| 30 | 1,80E-7 | 1,50E-7 |
| 31 | 4,00E-7 | 1,50E-7 |
| 32 | 3.33E-7 | 2,21E-6 |
| 33 | 2.60E-8 | 1.30E-7 |
| 34 | 2,15E-6 | 2,35E-6 |
| 35 | 7,00E-8 | 2.70E-7 |
| 36 | 9,00E-8 | 2,90E-7 |
| 37 | 3.50E-6 | 5.50E-6 |
| 38 | 2.83E-7 | 1.67E-6 |
| 39 | 7.23E-7 | 4.30E-6 |
| 40 | 4.45E-7 | 2.20E-6 |
| 41 | 5.07E-7 | 5.20E-6 |
| 42 | 7.25E-7 | 1,00E-5 |
| 43 | 5.50E-6 | 5,00E-5 |
| 44 | 5,50E-7 | 6.50E-6 |
| 45 | 5,54E-7 | 1,80 E-5 |
| | | 3,70 E-6 |
| | | >3,00 E-5 |
| | | 1,50 E-6 |
| 46 | 6,75E-7 | 6,00E-6 |
| 47 | 6.50E-7 | 3,20E-6 |
| 48 | 6.97E-7 | 4.15E-6 |
| 49 | 6.98E-7 | 4,93E-6 |
| 50 | 7.50E-7 | 5,00E-6 |
| 51 | 7,92E-7 | 4,33E-6 |
| 52 | 1.35E-6 | >3,00 E-5 |
| | | 4,50 E-6 |
| | | 4,80 E-6 |
| | | 6,50 E-6 |
| 53 | 7.50E-7 | 1,60E-6 |
| 54 | 1.80E-6 | 1,55E-5 |
| 55 | 9.25E-7 | 4,00E-6 |
| 56 | 1,17E-6 | 2,70E-6 |
| 57 | 1.50E-6 | 5,00E-6 |
| 58 | 1.80E-6 | n.d. |
| 59 | 1.90E-6 | 1,60E-5 |
| 60 | 7.50E-6 | n.d. |
| 61 | 2.50E-6 | n.d. |
| 62 | 2.50E-6 | n.d. |
| 63 | 8,00E-7 | 7,00E-6 |
| 64 | 2.97E-7 | 2,60E-6 |
| 65 | 5,00E-7 | 5.50E-6 |
| 66 | 6.70E-6 | 4.90E-5 |
| 67 | 5.10E-6 | n.d. |
| 68 | 2.16E-6 | 3,00E-5 |
| 69 | 4.50E-6 | n.d. |
| 70 | 1.33E-6 | 5,00E-5 |
| 71 | 1.75E-6 | 3,00E-5 |
| 72 | 2,00E-6 | 2.50E-5 |
| 73 | 9,00E-7 | 5.90E-6 |
| 74 | 4.60E-7 | 2.45E-6 |
| 75 | 2.70E-6 | 1.60E-5 |
| 76 | 5.99E-8 | 4.52E-7 |
| 77 | 1,60E-7 | 7,70E-7 |
| 78 | 1.50E-8 | 1,40E-7 |
| 79 | 7,00E-7 | 4,00E-6 |
| 80 | 3,00E-7 | 2.50E-6 |
| 81 | 2.50E-6 | 1.60E-5 |
| AE1* | 4.60E-6 | 1.90E-5 |
| AE2* | 2.30E-6 | n.d. |
| AE3* | 4,00E-6 | 1.20E-5 |

*AE = Alternative Example

TABLE 7
IC₅₀ values of examples in in vitro assay 2

| Example | MDAMB-231 IC₅₀ [M] | U87-MG IC₅₀ [M] |
|---|---|---|
| 1 | 1.80E-5 | 6,25E-6 |
| 2 | 2.50E-5 | n.d. |
| 3 | >3,00E-5 | 1,11E-5 |
| 4 | >3,00 E-5 | 1,20E-5 |
| | 5,00 E-5 | |
| 5 | 2,50 E-5 | 6,00E-6 |
| | 8,50 E-6 | |
| | >3,00 E-5 | |
| 6 | >3,00 E-5 | n.d. |
| | >3,00 E-5 | |
| 7 | 3,00 E-5 | 1,02E-5 |
| | 3,00 E-5 | |
| | >3,00 E-5 | |
| 8 | n.d. | 6,00E-6 |
| 9 | n.d. | 1,50E-6 |
| 10 | 3,00 E-5 | 1,30E-5 |
| | 3,00 E-5 | |
| | >3,00 E-5 | |
| 11 | 1,41 E-5 | 3,45E-6 |
| 12 | n.d. | n.d. |
| 13 | 6,30E-6 | 3.28E-6 |
| 14 | 1,04E-5 | 2,70E-6 |
| 15 | 1,70E-5 | 2,00E-5 |
| 16 | 1,08E-5 | 5,40E-6 |
| 17 | n.d. | n.d. |
| 18 | >3,00E-5 | 4,00E-6 |
| 19 | >3,00 E-5 | 6,30E-6 |
| | >3,00 E-5 | |
| 20 | 2,70E-5 | 2,20E-5 |
| 21 | >3,00 E-5 | 1.14E-5 |
| | 4,20 E-6 | |
| 22 | >3,00E-5 | n.d. |
| 23 | 3,00 E-5 | n.d. |
| | >3,00 E-5 | |
| 24 | 3,47E-6 | 7,33E-7 |
| 25 | 2.30E-5 | n.d. |
| 26 | n.d. | n.d. |
| 27 | >3,00 E-5 | 6,50E-6 |
| | 2,00 E-5 | |
| 28 | n.d. | 6,20E-6 |
| 29 | n.d. | n.d. |
| 30 | n.d. | n.d. |
| 31 | n.d. | n.d. |
| 32 | 1,80E-6 | 1,16E-6 |
| 33 | 8,74E-7 | 3,58E-7 |
| 34 | n.d. | 2,00E-6 |
| 35 | 4,80E-6 | 5,00E-6 |
| 36 | 2,70E-6 | 7.80E-7 |
| 37 | n.d. | 4,70E-6 |
| 38 | n.d. | n.d. |
| 39 | 2,00E-5 | 3,00E-6 |
| 40 | 1,20E-5 | 4.63E-6 |
| 41 | >3,00E-5 | n.d. |
| 42 | >3,00 E-5 | >3,00E-5 |
| | >3,00 E-5 | |
| 43 | n.d. | >3,00E-5 |
| 44 | n.d. | >3,00E-5 |
| 45 | 3,00E-5 | n.d. |
| 46 | >3,00E-5 | 2,50E-5 |
| 47 | >3,00E-5 | n.d. |
| 48 | 2,50E-5 | n.d. |
| 49 | >3,00E-5 | n.d. |
| 50 | n.d. | 3,60E-6 |
| 51 | 3,50 E-5 | n.d. |
| | 1,50 E-5 | |
| | >3,00 E-5 | |
| 52 | 3,00E-5 | 2,95E-6 |
| 53 | n.d. | n.d. |
| 54 | n.d. | n.d. |
| 55 | n.d. | 6,00E-6 |
| 56 | 2,74E-6 | 2,35E-6 |
| 57 | n.d. | n.d. |
| 58 | n.d. | n.d. |
| 59 | n.d. | n.d. |
| 60 | n.d. | n.d. |
| 61 | n.d. | n.d. |
| 62 | n.d. | n.d. |
| 63 | >3,00 E-5 | 2,00E-5 |
| | >3,00 E-5 | |
| 64 | n.d. | n.d. |
| 65 | >3,00E-5 | n.d. |
| 66 | n.d. | n.d. |
| 67 | n.d. | n.d. |
| 68 | 9,00E-6 | 7,50E-6 |
| 69 | >3,00E-5 | >3,00 E-5 |
| | | >3,00 E-5 |
| 70 | >3,00E-5 | 3,00 E-5 |
| | | >3,00 E-5 |
| 71 | >3,00E-5 | >3,00 E-5 |
| | | >3,00 E-5 |
| 72 | >3,00E-5 | n.d. |
| 73 | >3,00E-5 | >3,00 E-5 |
| | | >3,00 E-5 |
| 74 | >3,00 E-5 | 2,20E-5 |
| | 3,00 E-5 | |
| 75 | n.d. | n.d. |
| 76 | >3,00 E-5 | >3,00 E-5 |
| | >3,00 E-5 | >3,00 E-5 |
| | >3,00 E-5 | 1,20 E-5 |
| | 2,00 E-5 | |
| 77 | 1.60E-5 | 1,65E-5 |
| 78 | 3,80E-6 | 1.25E-5 |
| 79 | >3,00 E-5 | n.d. |
| | >3,00 E-5 | |
| 80 | n.d. | n.d. |
| 81 | n.d. | n.d. |
| AE1* | >3,00 E-5 | 1,50E-5 |
| AE2* | n.d. | n.d. |
| AE3* | 3,00E-5 | n.d |

*AE = Alternative Example

1. A compound of general formula (I): 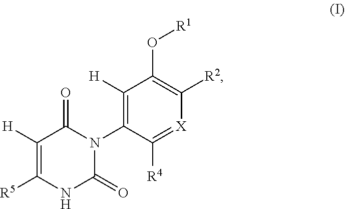
wherein:
R¹ is a group 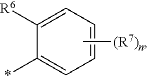
wherein “*” is the point of attachment to the rest of the molecule,
R² is a chlorine atom or a cyano group,
X is CR³ or N,
R³ is a hydrogen atom,
R⁴ is a hydrogen atom, a halogen atom or a group selected from hydroxy, C₁-C₃-alkyl, C₁-C₃-alkoxy and C₁-C₃-haloalkoxy,
or R³ and R⁴ together form a group —CH═CH—CH═CH—,
R⁵ is a halogen atom or a group selected from C₁-C₃-haloalkyl, C₃-C₆-cycloalkyl, phenyl, —C(═O)NR⁸R⁹ and —S(═O)₂R¹⁰,
said C₁-C₃-haloalkyl group being optionally substituted with one phenyl group,
and said phenyl group being optionally substituted with one or two substituents selected independently from each other from a halogen atom and C₁-C₃-halo alkyl,
R⁶ is a fluorine atom, a chlorine atom, or a methyl group,
R⁷ is a halogen atom or a group selected from amino, cyano, C₁-C₃-alkyl or H₃C—C(═O)—,
R⁸ is a hydrogen atom or a group selected from C₁-C₃-alkyl and C₁-C₃-haloalkyl,
R⁹ is a hydrogen atom or a methyl group,
R¹⁰ is a group selected from C₁-C₃-alkyl and phenyl, said phenyl group being optionally substituted with one or two substituents selected independently from each other from a halogen atom, C₁-C₃-alkyl, C₁-C₃-haloalkyl, C₁-C₃-alkoxy and cyano, and
n is an integer 0, 1 or 2,
or a stereoisomer, a tautomer, an N-oxide, a hydrate, a solvate, or a salt thereof, or a mixture of same,
with the proviso that said compound of formula (I) is not:
4-[2,6-Dioxo-4-(trifluoromethyl)-3,6-dihydropyrimidin-1(2H)-yl]-5-fluoro-2-(2-fluorophenoxy)benzonitrile 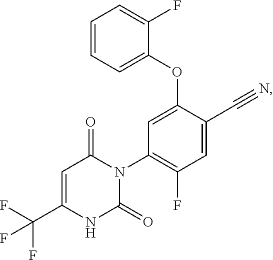 [SMILES: N#Cc1cc(F)c(-n2c(=O)cc(C(F)(F)F)[nH]c2=O)cc1Oc1ccccc1F]
2-(2,4-Difluorophenoxy)-4-[2,6-dioxo-4-(trifluoromethyl)-3,6-dihydropyrimidin-1(2H)-yl]-5-fluorobenzonitrile 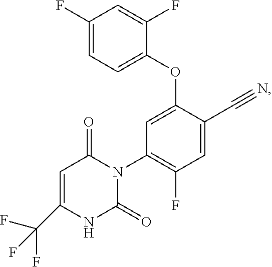 [SMILES: N#Cc1cc(F)c(-n2c(=O)cc(C(F)(F)F)[nH]c2=O)cc1Oc1ccc(F)cc1F]
or
2-(2-Chlorophenoxy)-4-[2,6-dioxo-4-(trifluoromethyl)-3,6-dihydropyrimidin-1(2H)-yl]-5-fluorobenzonitrile

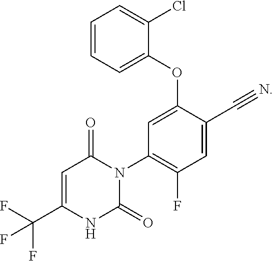

2. The compound according to claim 1, wherein:
R¹ is a group 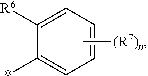
wherein “*” is the point of attachment to the rest of the molecule,
R² is a chlorine atom or a cyano group,
X is CR³,
R³ is a hydrogen atom,
R⁴ is a hydrogen atom, a halogen atom or a group selected from hydroxy, C₁-C₂-alkyl, C₁-C₃-alkoxy and C₁-C₂-fluoroalkoxy,
or R³ and R⁴ together form a group —CH═CH—CH═CH—;
R⁵ is a halogen atom or a group selected from C₁-C₂-haloalkyl, C₃-C₄-cycloalkyl, phenyl, —C(═O)NR⁸R⁹ and —S(═O)₂R¹⁰,
said C₁-C₂-haloalkyl group being optionally substituted with one phenyl group,
and said phenyl group being optionally substituted with one or two substituents selected independently from each other from a fluorine atom, a chlorine atom, a bromine atom and C₁-C₂-fluoroalkyl,
R⁶ is a fluorine atom, a chlorine atom, or a methyl group,
R⁷ is a halogen atom or a group selected from amino, cyano, C₁-C₃-alkyl or H₃C—C(═O)—,
R⁸ is a hydrogen atom or a group selected from C₁-C₂-alkyl and C₁-C₂-fluoroalkyl,
R⁹ is a hydrogen atom,
R¹⁰ is a group selected from C₁-C₂-alkyl and phenyl, said phenyl group being optionally substituted with one or two substituents selected independently from each other from a fluorine atom, a chlorine atom, a bromine atom, C₁-C₂-alkyl, C₁-C₂-fluoroalkyl and C₁-C₂-alkoxy, and
n is an integer 0, 1 or 2,
or a stereoisomer, a tautomer, an N-oxide, a hydrate, a solvate, or a salt thereof, or a mixture of same,
with the proviso that said compound of formula (I) is not:
4-[2,6-Dioxo-4-(trifluoromethyl)-3,6-dihydropyrimidin-1(2H)-yl]-5-fluoro-2-(2-fluorophenoxy)benzonitrile,
2-(2,4-Difluorophenoxy)-4-[2,6-dioxo-4-(trifluoromethyl)-3,6-dihydropyrimidin-1(2H)-yl]-5-fluorobenzonitrile, or
2-(2-Chlorophenoxy)-4-[2,6-dioxo-4-(trifluoromethyl)-3,6-dihydropyrimidin-1(2H)-yl]-5-fluorobenzonitrile.

3. The compound according to claim 1, wherein:
R¹ is a group 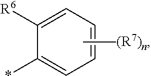
wherein “*” is the point of attachment to the rest of the molecule,
R² is a chlorine atom or a cyano group,
X is CR³,
R³ and R⁴ together form a group —CH═CH—CH═CH—
R⁵ is a halogen atom or a group selected from C₁-C₂-haloalkyl, C₃-C₄-cycloalkyl, phenyl, —C(═O)NR⁸R⁹ and —S(═O)₂R¹⁰,
said C₁-C₂-haloalkyl group being optionally substituted with one phenyl group,
and said phenyl group being optionally substituted with one or two substituents selected independently from each other from a fluorine atom, a chlorine atom, a bromine atom and C₁-C₂-fluoroalkyl,
R⁶ is a fluorine atom, a chlorine atom, or a methyl group,
R⁷ is a halogen atom or a group selected from amino, cyano, C₁-C₃-alkyl or H₃C—C(═O)—,
R⁸ is a hydrogen atom or a group selected from C₁-C₂-alkyl and C₁-C₂-fluoroalkyl,
R⁹ is a hydrogen atom,
R¹⁰ is a group selected from C₁-C₂-alkyl and phenyl, said phenyl group being optionally substituted with one or two substituents selected independently from each other from a fluorine atom, a chlorine atom, a bromine atom, C₁-C₂-alkyl, C₁-C₂-fluoroalkyl and C₁-C₂-alkoxy, and
n is an integer 0, 1 or 2,
or a stereoisomer, a tautomer, an N-oxide, a hydrate, a solvate, or a salt thereof, or a mixture of same.

4. The compound according to claim 1, wherein:
R¹ is a group 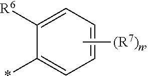
wherein “*” is the point of attachment to the rest of the molecule,
R² is a chlorine atom or a cyano group,
X is CR³,
R³ is a hydrogen atom,
R⁴ is a halogen atom or a group selected from methyl, methoxy, trifluoromethoxy and n-propoxy,
or R³ and R⁴ together form a group —CH═CH—CH═CH—,
R⁵ is a bromine atom or a group selected from difluoromethyl, (phenyl)-difluoromethyl, chlorodifluoromethyl, trifluoromethyl, cyclopropyl, methanesulfonyl and benzenesulfonyl,
R⁶ is a fluorine atom, a chlorine atom, or a methyl group,
R⁷ is a halogen atom or a group selected from amino, cyano, C₁-C₃-alkyl or H₃C—C(═O)—, and
n is an integer 0, 1 or 2,
or a stereoisomer, a tautomer, an N-oxide, a hydrate, a solvate, or a salt thereof, or a mixture of same,
with the proviso that said compound of formula (I) is not:
4-[2,6-Dioxo-4-(trifluoromethyl)-3,6-dihydropyrimidin-1(2H)-yl]-5-fluoro-2-(2-fluorophenoxy)benzonitrile,
2-(2,4-Difluorophenoxy)-4-[2,6-dioxo-4-(trifluoromethyl)-3,6-dihydropyrimidin-1(2H)-yl]-5-fluorobenzonitrile, or
2-(2-Chlorophenoxy)-4-[2,6-dioxo-4-(trifluoromethyl)-3,6-dihydropyrimidin-1(2H)-yl]-5-fluorobenzonitrile.

5. The compound according to claim 1, wherein:
R¹ is a group 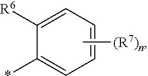
wherein “*” is the point of attachment to the rest of the molecule,
R² is a chlorine atom or a cyano group,
X is CR³,
R³ and R⁴ together form a group —CH═CH—CH═CH—,
R⁵ is a bromine atom or a group selected from difluoromethyl, (phenyl)-difluoromethyl, chlorodifluoromethyl, trifluoromethyl, cyclopropyl, methanesulfonyl and benzenesulfonyl,
R⁶ is a fluorine atom, a chlorine atom, or a methyl group,
R⁷ is a halogen atom or a group selected from amino, cyano, C₁-C₃-alkyl or H₃C—C(═O)—, and
n is an integer 0, 1 or 2,
or a stereoisomer, a tautomer, an N-oxide, a hydrate, a solvate, or a salt thereof, or a mixture of same.

6. The compound according to claim 1, which is selected from the group consisting of:
2-(2,6-Dimethylphenoxy)-4-[2,6-dioxo-4-(trifluoromethyl)-3,6-dihydropyrimidin-1(2H)-yl]-5-fluorobenzonitrile,
2-(2-Chloro-6-methylphenoxy)-4-[2,6-dioxo-4-(trifluoromethyl)-3,6-dihydropyrimidin-1(2H)-yl]-5-fluorobenzonitrile,
4-(4-Bromo-2,6-dioxo-3,6-dihydropyrimidin-1(2H)-yl)-5-fluoro-2-(2-methylphenoxy)benzonitrile,
4-[2,6-Dioxo-4-(trifluoromethyl)-3,6-dihydropyrimidin-1(2H)-yl]-5-methoxy-2-(2-methylphenoxy)benzonitrile (rac),
4-[2,6-Dioxo-4-(trifluoromethyl)-3,6-dihydropyrimidin-1(2H)-yl]-5-methoxy-2-(2-methylphenoxy)benzonitrile (atrop 1),
4-[2,6-Dioxo-4-(trifluoromethyl)-3,6-dihydropyrimidin-1(2H)-yl]-5-methoxy-2-(2-methylphenoxy)benzonitrile (atrop 2),
4-[2,6-Dioxo-4-(trifluoromethyl)-3,6-dihydropyrimidin-1(2H)-yl]-5-methyl-2-(2-methylphenoxy)benzonitrile (rac),
4-[2,6-Dioxo-4-(trifluoromethyl)-3,6-dihydropyrimidin-1(2H)-yl]-5-methyl-2-(2-methylphenoxy)benzonitrile (atrop 1),
4-[2,6-Dioxo-4-(trifluoromethyl)-3,6-dihydropyrimidin-1(2H)-yl]-5-methyl-2-(2-methylphenoxy)benzonitrile (atrop 2),
2-(2-Chlorophenoxy)-4-[2,6-dioxo-4-(trifluoromethyl)-3,6-dihydropyrimidin-1(2H)-yl]-5-methylbenzonitrile (rac),
5-Chloro-4-[2,6-dioxo-4-(trifluoromethyl)-3,6-dihydropyrimidin-1(2H)-yl]-2-(2-methylphenoxy)benzonitrile (rac),
5-Chloro-4-[2,6-dioxo-4-(trifluoromethyl)-3,6-dihydropyrimidin-1(2H)-yl]-2-(2-methylphenoxy)benzonitrile (atrop 1),
5-Chloro-4-[2,6-dioxo-4-(trifluoromethyl)-3,6-dihydropyrimidin-1(2H)-yl]-2-(2-methylphenoxy)benzonitrile (atrop 2),
4-[2,6-Dioxo-4-(trifluoromethyl)-3,6-dihydropyrimidin-1(2H)-yl]-5-iodo-2-(2-methylphenoxy)benzonitrile (rac),
2-(2-Chlorophenoxy)-4-[2,6-dioxo-4-(trifluoromethyl)-3,6-dihydropyrimidin-1(2H)-yl]-5-methoxybenzonitrile (rac),
2-(2-Chlorophenoxy)-4-[2,6-dioxo-4-(trifluoromethyl)-3,6-dihydropyrimidin-1(2H)-yl]-5-methoxybenzonitrile (atrop 1),
2-(2-Chlorophenoxy)-4-[2,6-dioxo-4-(trifluoromethyl)-3,6-dihydropyrimidin-1(2H)-yl]-5-methoxybenzonitrile (atrop 2),
5-Bromo-4-[2,6-dioxo-4-(trifluoromethyl)-3,6-dihydropyrimidin-1(2H)-yl]-2-(2-methylphenoxy)benzonitrile (rac),
4-[4-Bromo-2,6-dioxo-3,6-dihydropyrimidin-1(2H)-yl]-5-methoxy-2-(2-methylphenoxy)benzonitrile (rac),
4-[4-Bromo-2,6-dioxo-3,6-dihydropyrimidin-1(2H)-yl]-5-methoxy-2-(2-methylphenoxy)benzonitrile (atrop 1),
4-[4-Bromo-2,6-dioxo-3,6-dihydropyrimidin-1(2H)-yl]-5-methoxy-2-(2-methylphenoxy)benzonitrile (atrop 2),
4-[4-(Difluoromethyl)-2,6-dioxo-3,6-dihydropyrimidin-1(2H)-yl]-5-methoxy-2-(2-methylphenoxy)benzonitrile (rac),
4-(4-Cyclopropyl-2,6-dioxo-3,6-dihydropyrimidin-1(2H)-yl)-5-methoxy-2-(2-methylphenoxy)benzonitrile (rac),
4-{4-[Difluoro(phenyl)methyl]-2,6-dioxo-3,6-dihydropyrimidin-1(2H)-yl}-5-methoxy-2-(2-methylphenoxy)benzonitrile (rac),
4-{4-[Chloro(difluoro)methyl]-2,6-dioxo-3,6-dihydropyrimidin-1(2H)-yl}-5-methoxy-2-(2-methylphenoxy)benzonitrile (rac),
5-Chloro-4-{4-[chloro(difluoro)methyl]-2,6-dioxo-3,6-dihydropyrimidin-1(2H)-yl}-2-(2-methylphenoxy)benzonitrile (rac),
3-[4-Chloro-2-methoxy-5-(2-methylphenoxy)phenyl]-6-(trifluoromethyl)pyrimidine-2,4(1H,3H)-dione (rac),
3-[4-Chloro-2-methoxy-5-(2-methylphenoxy)phenyl]-6-(difluoromethyl)pyrimidine-2,4(1H,3H)-dione (rac),
3-[4-Chloro-3-(2-methylphenoxy)naphthalen-1-yl]-6-[difluoro(phenyl)methyl]pyrimidine-2,4(1H,3H)-dione (rac),
3-[4-chloro-3-(2-methylphenoxy)naphthalen-1-yl]-6-[difluoro(phenyl)methyl]pyrimidine-2,4(1H,3H)-dione (atrop 1),
3-[4-chloro-3-(2-methylphenoxy)naphthalen-1-yl]-6-[difluoro(phenyl)methyl]pyrimidine-2,4(1H,3H)-dione (atrop 2),
3-[4-Chloro-3-(2-methylphenoxy)naphthalen-1-yl]-6-(trifluoromethyl)pyrimidine-2,4(1H,3H)-dione (rac),
3-[4-Chloro-3-(2-methylphenoxy)naphthalen-1-yl]-6-(trifluoromethyl)pyrimidine-2,4(1H,3H)-dione (atrop 1),
3-[4-Chloro-3-(2-methylphenoxy)naphthalen-1-yl]-6-(trifluoromethyl)pyrimidine-2,4(1H,3H)-dione (atrop 2),
4-[2,6-Dioxo-4-(trifluoromethyl)-3,6-dihydropyrimidin-1(2H)-yl]-2-(2-methylphenoxy)naphthalene-1-carbonitrile (rac),
4-[2,6-Dioxo-4-(trifluoromethyl)-3,6-dihydropyrimidin-1(2H)-yl]-2-(2-methylphenoxy)naphthalene-1-carbonitrile (atrop 1),
4-[2,6-Dioxo-4-(trifluoromethyl)-3,6-dihydropyrimidin-1(2H)-yl]-2-(2-methylphenoxy)naphthalene-1-carbonitrile (atrop 2),
5-Chloro-2-(2,6-dimethylphenoxy)-4-(2,6-dioxo-4-(trifluoromethyl)-2,3-dihydropyrimidin-1(6H)-yl)benzonitrile (rac),
4-(2,6-Dioxo-4-(trifluoromethyl)-2,3-dihydropyrimidin-1(6H)-yl)-5-propoxy-2-(o-tolyloxy)benzonitrile (rac),
4-{4-[Chloro(difluoro)methyl]-2,6-dioxo-3,6-dihydropyrimidin-1(2H)-yl}-5-fluoro-2-(2-methylphenoxy)benzonitrile,
4-[2,6-Dioxo-4-(trifluoromethyl)-3,6-dihydropyrimidin-1(2H)-yl]-2-(2-methylphenoxy)benzonitrile,
4-(2,6-Dioxo-4-phenyl-3,6-dihydropyrimidin-1(2H)-yl)-5-methoxy-2-(2-methylphenoxy)benzonitrile (rac),
4-(2,6-Dioxo-4-phenyl-3,6-dihydropyrimidin-1(2H)-yl)-5-methoxy-2-(2-methylphenoxy)benzonitrile (atrop 1),
4-(2,6-Dioxo-4-phenyl-3,6-dihydropyrimidin-1(2H)-yl)-5-methoxy-2-(2-methylphenoxy)benzonitrile (atrop 2),
4-[2,6-Dioxo-4-(trifluoromethyl)-3,6-dihydropyrimidin-1(2H)-yl]-5-fluoro-2-(2-methylphenoxy)benzonitrile,
2-(2-Chlorophenoxy)-4-[2,6-dioxo-4-(trifluoromethyl)-3,6-dihydropyrimidin-1(2H)-yl]benzonitrile,
5-[2,6-Dioxo-4-(trifluoromethyl)-3,6-dihydropyrimidin-1(2H)-yl]-3-(2-methylphenoxy)pyridine-2-carbonitrile,
2-(2,6-Dimethylphenoxy)-4-[2,6-dioxo-4-(trifluoromethyl)-3,6-dihydropyrimidin-1(2H)-yl]benzonitrile,
4-(2,6-Dioxo-4-phenyl-3,6-dihydropyrimidin-1(2H)-yl)-5-fluoro-2-(2-methylphenoxy)benzonitrile,
3-[4-Chloro-5-(2-chlorophenoxy)-2-methoxyphenyl]-6-(trifluoromethyl)pyrimidine-2,4(1H,3H)-dione (rac),
4-[2,6-Dioxo-4-(pentafluoroethyl)-3,6-dihydropyrimidin-1(2H)-yl]-5-methoxy-2-(2-methylphenoxy)benzonitrile (rac),
4-[2,6-dioxo-4-(pentafluoroethyl)-3,6-dihydropyrimidin-1(2H)-yl]-5-fluoro-2-(2-methylphenoxy)benzonitrile,
5-Chloro-4-{4-[difluoro(phenyl)methyl]-2,6-dioxo-3,6-dihydropyrimidin-1(2H)-yl}-2-(2-methylphenoxy)benzonitrile (rac),
4-[2,6-Dioxo-4-(trifluoromethyl)-3,6-dihydropyrimidin-1(2H)-yl]-5-hydroxy-2-(2-methylphenoxy)benzonitrile (rac),
4-[2,6-Dioxo-4-(trifluoromethyl)-3,6-dihydropyrimidin-1(2H)-yl]-2-(2-methylphenoxy)-5-(trifluoromethoxy)benzonitrile (rac),
4-{4-[Difluoro(phenyl)methyl]-2,6-dioxo-3,6-dihydropyrimidin-1(2H)-yl}-5-fluoro-2-(2-methylphenoxy)benzonitrile,
4-{4-[Difluoro(phenyl)methyl]-2,6-dioxo-3,6-dihydropyrimidin-1(2H)-yl}-2-(2,6-dimethylphenoxy)-5-fluorobenzonitrile,
2-(2-Acetyl-4,6-dimethylphenoxy)-4-[2,6-dioxo-4-(trifluoromethyl)-3,6-dihydropyrimidin-1(2H)-yl]-5-fluorobenzonitrile,
4-[2,6-Dioxo-4-(trifluoromethyl)-3,6-dihydropyrimidin-1(2H)-yl]-2-(2-ethyl-6-methylphenoxy)-5-fluorobenzonitrile,
4-[2,6-Dioxo-4-(trifluoromethyl)-3,6-dihydropyrimidin-1(2H)-yl]-5-fluoro-2-(2,3,6-trimethylphenoxy)benzonitrile,
2-(4-Chloro-2,6-dimethylphenoxy)-4-[2,6-dioxo-4-(trifluoromethyl)-3,6-dihydropyrimidin-1(2H)-yl]-5-fluorobenzonitrile,
4-[2,6-Dioxo-4-(trifluoromethyl)-3,6-dihydropyrimidin-1(2H)-yl]-5-fluoro-2-(2,4,6-trimethylphenoxy)benzonitrile,
2-(2,6-Difluorophenoxy)-4-[2,6-dioxo-4-(trifluoromethyl)-3,6-dihydropyrimidin-1(2H)-yl]-5-fluorobenzonitrile,
2-(2-Amino-6-fluorophenoxy)-4-[2,6-dioxo-4-(trifluoromethyl)-3,6-dihydropyrimidin-1(2H)-yl]-5-fluorobenzonitrile,
2-(2-Amino-6-methylphenoxy)-4-[2,6-dioxo-4-(trifluoromethyl)-3,6-dihydropyrimidin-1(2H)-yl]-5-fluorobenzonitrile,
2-(2,4-Dimethylphenoxy)-4-[2,6-dioxo-4-(trifluoromethyl)-3,6-dihydropyrimidin-1(2H)-yl]-5-fluorobenzonitrile,
2-(2,3-Dimethylphenoxy)-4-[2,6-dioxo-4-(trifluoromethyl)-3,6-dihydropyrimidin-1(2H)-yl]-5-fluorobenzonitrile,
4-{2,6-Dioxo-4-[4-(trifluoromethyl)phenyl]-3,6-dihydropyrimidin-1(2H)-yl}-5-fluoro-2-(2-methylphenoxy)benzonitrile,
4-{2,6-Dioxo-4-[4-(trifluoromethyl)phenyl]-3,6-dihydropyrimidin-1(2H)-yl}-5-methoxy-2-(2-methylphenoxy)benzonitrile (rac),
4-[4-(3-Chlorophenyl)-2,6-dioxo-3,6-dihydropyrimidin-1(2H)-yl]-5-fluoro-2-(2-methylphenoxy)benzonitrile,
1-[4-Cyano-2-fluoro-5-(2-methylphenoxy)phenyl]-2,6-dioxo-N-(2,2,2-trifluoroethyl)-1,2,3,6-tetrahydropyrimidine-4-carboxamide,
4-(4-Cyclopropyl-2,6-dioxo-3,6-dihydropyrimidin-1(2H)-yl)-5-fluoro-2-(2-methylphenoxy)benzonitrile,
1-[4-Cyano-2-methoxy-5-(2-methylphenoxy)phenyl]-2,6-dioxo-1,2,3,6-tetrahydropyrimidine-4-carboxamide (rac),
4-[4-(Benzenesulfonyl)-2,6-dioxo-3,6-dihydropyrimidin-1(2H)-yl]-5-fluoro-2-(2-methylphenoxy)benzonitrile,
5-Fluoro-4-[4-(methanesulfonyl)-2,6-dioxo-3,6-dihydropyrimidin-1(2H)-yl]-2-(2-methylphenoxy)benzonitrile,
4-[4-(Benzenesulfonyl)-2,6-dioxo-3,6-dihydropyrimidin-1(2H)-yl]-5-methoxy-2-(2-methylphenoxy)benzonitrile (rac),
4-[4-(Benzenesulfonyl)-2,6-dioxo-3,6-dihydropyrimidin-1(2H)-yl]-5-methoxy-2-(2-methylphenoxy)benzonitrile (atrop 1),
4-[4-(Benzenesulfonyl)-2,6-dioxo-3,6-dihydropyrimidin-1(2H)-yl]-5-methoxy-2-(2-methylphenoxy)benzonitrile (atrop 2),
4-[4-(Methanesulfonyl)-2,6-dioxo-3,6-dihydropyrimidin-1(2H)-yl]-5-methoxy-2-(2-methylphenoxy)benzonitrile (rac),
4-[4-(Methanesulfonyl)-2,6-dioxo-3,6-dihydropyrimidin-1(2H)-yl]-5-methoxy-2-(2-methylphenoxy)benzonitrile (atrop 1), and
4-[4-(Methanesulfonyl)-2,6-dioxo-3,6-dihydropyrimidin-1(2H)-yl]-5-methoxy-2-(2-methylphenoxy)benzonitrile (atrop 2),
or a stereoisomer, a tautomer, an N-oxide, a hydrate, a solvate, or a salt thereof, or a mixture of same.

7. A method of preparing a compound of formula (I) according to claim 1, comprising reacting an intermediate compound of formula (III) 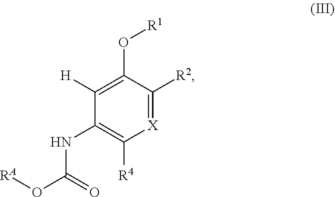
wherein R¹, R², R⁴ and X are as defined for the compound of 1 formula (I) according to claim 1, and
R^{A} is a C₁-C₄-alkyl group,
with a mixture, previously prepared from sodium hydride and an acrylate ester of the formula R⁵—C(NH₂)═C(H)—C(═O)—OR^{A′} in a dipolar aprotic solvent, wherein R⁵ is as defined for the compound of formula (I) according to claim 1, and wherein R^{A′} is a C₁-C₄-alkyl group,
thereby giving a compound of formula (I):

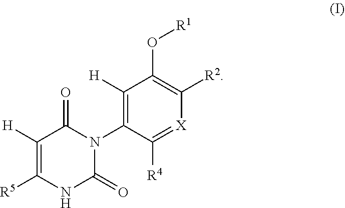

8. A method for treatment or prophylaxis of a disease, comprising administering a compound of formula (I) according to claim 1 to a mammal in need thereof.

9. A pharmaceutical composition comprising a compound of formula (I) according to claim 1, and one or more pharmaceutically acceptable excipients.

10. A pharmaceutical combination comprising:
one or more compounds of formula (I) according to claim 1; and
one or more further active ingredients selected from:
131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, 1-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, or zorubicin.

11. (canceled)

12. (canceled)

13. The method of claim 8, wherein the disease is cancer.

14. A compound of formula (III): 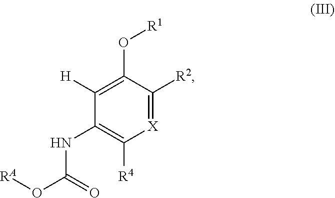
wherein
R¹ is a group 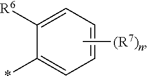
wherein “*” is the point of attachment to the rest of the molecule,
R² is a chlorine atom or a cyano group,
X is CR³ or N,
R³ is a hydrogen atom,
R⁴ is a hydrogen atom, a halogen atom or a group selected from hydroxy, C₁-C₃-alkyl, C₁-C₃-alkoxy and C₁-C₃-haloalkoxy,
or R³ and R⁴ together form a group —CH═CH—CH═CH—, and
R^{A} is a C₁-C₄-alkyl group.

15. (canceled)

16. The method of claim 13, wherein the cancer is glioma, lung cancer, breast cancer, leukemia, prostate cancer, ovarian cancer, urothelial carcinoma, gastric cancer or nasopharyngeal carcinoma.

17. The method of claim 16, wherein the cancer is glioma or breast cancer.

18. The method of claim 16, wherein the cancer is non-small-cell lung cancer (NSCLC), anti-estrogen resistant breast cancer or ERalpha-negative breast cancer.

19. The method of claim 8, wherein the disease is cancer-induced cachexia or fibrosis.
